SEQUENCE LISTING (79 sequences)

SEQ ID NO: 1 - DNA, 1059 bp, Saccharomyces cerevisiae
  CDS (1)..(1056)
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatag

SEQ ID NO: 2 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 3 - DNA, 900 bp, Escherichia coli
  CDS (1)..(897)
atggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataa

SEQ ID NO: 4 - protein, 299 aa, Escherichia coli
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 5 - DNA, 1008 bp, Saccharomyces cerevisiae
  CDS (1)..(1005)
atggaggccaagatagatgagctgatcaataatgatcctgtttggtccagccaaaatgaaagcttgatttcaaaaccttataatcacatccttttgaaacctggcaagaactttagactaaatttaatagttcaaattaacagagttatgaatttgcccaaagaccagctggccatagtttcgcaaattgttgagctcttgcataattccagccttttaatcgacgatatagaagataatgctcccttgagaaggggacagaccacttctcacttaatcttcggtgtaccctccactataaacaccgcaaattatatgtatttcagagccatgcaacttgtatcgcagctaaccacaaaagagcctttgtatcataatttgattacgattttcaacgaagaattgatcaatctacataggggacaaggcttggatatatactggagagactttctgcctgaaatcatacctactcaggagatgtatttgaatatggttatgaataaaacaggcggccttttcagattaacgttgagactcatggaagcgctgtctccttcctcacaccacggccattcgttggttcctttcataaatcttctgggtattatttatcagattagagatgattacttgaatttgaaagatttccaaatgtccagcgaaaaaggctttgctgaggacattacagaggggaagttatcttttcccatcgtccacgcccttaacttcactaaaacgaaaggtcaaactgagcaacacaatgaaattctaagaattctcctgttgaggacaagtgataaagatataaaactaaagctgattcaaatactggaattcgacaccaattcattggcctacaccaaaaattttattaatcaattagtgaatatgataaaaaatgataatgaaaataagtatttacctgatttggcttcgcattccgacaccgccaccaatttacatgacgaattgttatatataatagaccacttatccgaattgtga

SEQ ID NO: 6 - protein, 335 aa, Saccharomyces cerevisiae [sequence omitted]

SEQ ID NO: 7 - DNA, 3165 bp, Saccharomyces cerevisiae
  CDS (1)..(3162)
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagactccaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagtgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaatttttgaattgattttaactcctacattttattctgctatcttagcgcttagactggaaatgaatgttatccacagatctactattatcaagcaaacattagaagaagacggtgttgttccatctacagcaagaatcatttctaaagcagaaaagaaatccgtatcttctttcttaaatctcagtgtggttgtcattatcatgaaactctctgtcatactgttgtttgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattgaatgctgctagaattcataccagttatactgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 8 - protein, 1054 aa, Saccharomyces cerevisiae
MPPLFKGLKQMAKPIAYVSRFSAKRPIHIILFSLIISAFAYLSVIQYYFNGWQLDSNSVFETAPNKDSNTLFQECSHYYRDSSLDGWVSITAHEASELPAPHHYYLLNLNFNSPNETDSIPELANTVFEKDNTKYILQEDLSVSKEISSTDGTKWRLRSDRKSLFDVKTLAYSLYDVFSENVTQADPFDVLIMVTAYLMMFYTIFGLFNDMRKTGSNFWLSASTVVNSASSLFLALYVTQCILGKEVSALTLFEGLPFIVVVVGFKHKIKIAQYALEKFERVGLSKRITTDEIVFESVSEEGGRLIQDHLLCIFAFIGCSMYAHQLKTLTNFCILSAFILIFELILTPTFYSAILALRLEMNVIHRSTIIKQTLEEDGVVPSTARIISKAEKKSVSSFLNLSVVVIIMKLSVILLFVFINFYNFGANWVNDAFNSLYFDKERVSLPDFITSNASENFKEQAIVSVTPLLYYKPIKSYQRIEDMVLLLLRNVSVAIRDRFVSKLVLSALVCSAVINVYLLNAARIHTSYTADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 9 - DNA, 1191 bp, Saccharomyces cerevisiae
  CDS (1)..(1188)
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 10 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 11 - DNA, 3165 bp, Saccharomyces cerevisiae
  CDS (1)..(3162)
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagacttcaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagcgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaattttcgaattgattttaactcctacattttattctgctatcttagcgcttagactggaaatgaatgttatccacagatctactattatcaagcaaacattagaagaagacggtgttgttccatctacagcaagaatcatttctaaggcagaaaagaaatccgtatcttctttcttaaatctcagtgtggttgtcattatcatgaaactctctgtcatactgttgttcgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattaaatgctgctagaattcataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 12 - protein, 1054 aa, Saccharomyces cerevisiae
MPPLFKGLKQMAKPIAYVSRFSAKRPIHIILFSLIISAFAYLSVIQYYFNGWQLDSNSVFETAPNKDFNTLFQECSHYYRDSSLDGWVSITAHEASELPAPHHYYLLNLNFNSPNETDSIPELANTVFEKDNTKYILQEDLSVSKEISSTDGTKWRLRSDRKSLFDVKTLAYSLYDVFSENVTQADPFDVLIMVTAYLMMFYTIFGLFNDMRKTGSNFWLSASTVVNSASSLFLALYVTQCILGKEVSALTLFEGLPFIVVVVGFKHKIKIAQYALEKFERVGLSKRITTDEIVFESVSEEGGRLIQDHLLCIFAFIGCSMYAHQLKTLTNFCILSAFILIFELILTPTFYSAILALRLEMNVIHRSTIIKQTLEEDGVVPSTARIISKAEKKSVSSFLNLSVVVIIMKLSVILLFVFINFYNFGANWVNDAFNSLYFDKERVSLPDFITSNASENFKEQAIVSVTPLLYYKPIKSYQRIEDMVLLLLRNVSVAIRDRFVSKLVLSALVCSAVINVYLLNAARIHTSYTADQLVKTEVTKKSFTAPVQKASTPVLTNKTVISGSKVKSLSSAQSSSSGPSSSSEEDDSRDIESLDKKIRPLEELEASLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNARAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 13 - DNA, 3165 bp, Saccharomyces cerevisiae
  CDS (1)..(3162)
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagactccaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagcgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaattttcgaattgattttaactcctacattttattctgctatcttagcgcttagactggaaatgaatgttatccacagatctactattatcaagcaaacattagaagaagacggtgttgttccatctacagcaagaatcatttctaaggcagaaaagaaatccgtatcttctttcttaaatctcagtgtggttgtcattatcatgaaactctctgtcatactgttgttcgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattaaatgctgctagaattcataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 14 - protein, 1054 aa, Saccharomyces cerevisiae; identical to SEQ ID NO: 8

SEQ ID NO: 15 - DNA, 2925 bp, Saccharomyces cerevisiae
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagacttcaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagcgtttccaaagaaatttcttctactgatggaacgaaatggaggttaagaagtgacagaaaaagtcttttcgacgtaaagacgttagcatattctctctacgatgtattttcagaaaatgtaacccaagcagaccacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaattttcgaattgattttaactcctacattttattctgctatcttagcgcttagactggaaatgaatgttatccacagatctactattatcaagcaaacattagaagaagacggtgttgttccatctacagcaagaatcatttctaaggcagaaaagaaatccgtatcttctttcttaaatctcagtgtggttgtcattatcatgaaactctctgtcatactgttgttcgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattaaatgctgctagaattcataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 16 - DNA, 3090 bp, Saccharomyces cerevisiae
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagacttcaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagcgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaattttcgaattgattttaactcctacattttattctgctatcttagcgcttagactggaaatgaatgttatccacagatctactattatcaagcaaacattagaagaagacggtgttgttccatctacagcaagaatcatttctaaggcagaaaagaaatccgtatcttctaactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattaaatgctgctagaattcataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 17 - DNA, 2973 bp, Saccharomyces cerevisiae
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagacttcaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagcgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaattttcgaattgattttaactcctacattttattctgctatcttagcgcttagactggaaatgaatgttatccacagatctactattatcaagcaaacattagaagaagacggtgttgttccatctacagcaagaatcatttctaaggcagaaaagaaatccgtatcttctttcttaaatctcagtgtggttgtcattatcatgaaactctctgtcatactgttgttcgtcttcatcaacttttataactttggtgcaaattgggtcaatgatgccttcaattcattgtacttcgataaggaacgtgtttctctaccagattttattacctcgaatgcctctgaaaactttaaagagcaacataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 18 - DNA, 2457 bp, Saccharomyces cerevisiae
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagacttcaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagcgtttccaaagaaatttcttctactgatggaacgaaatggaggttaagaagtgacagaaaaagtcttttcgacgtaaagacgttagcatattctctctacgatgtattttcagaaaatgtaacccaagcagacaactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattaaatgctgctagaattcataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 19 - DNA, 2151 bp, Saccharomyces cerevisiae
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagacttcaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagcgtttccaaagaaatttcttctactgatggaacgaaatggaggttaagaagtgacagaaaaagtcttttcgacgtaaagacgttagcatattctctctacgatgtattttcagaaaatgtaacccaagcagaccataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 20 - DNA, 1620 bp, Saccharomyces cerevisiae
atgccgccgctattcaagggactgaaacataccagttatactgcagaccaattggtgaagactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 21 - DNA, 1377 bp, Saccharomyces cerevisiae
atgccgccgctattcaagggactgaaagcatcattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 22 - DNA, 1302 bp, Saccharomyces cerevisiae
atgccgccgctattcaagggactgaaacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 23 - DNA, 1203 bp, Saccharomyces cerevisiae
atgccgccgctattcaagggactgaaatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 24 - DNA, 975 bp, Saccharomyces cerevisiae
atgccgccgctattcaagggactgaaaaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacgtgcagctaatttagtgacagctgttttcttggcattaggacaagatcctgcacaaaatgtcgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 25 - DNA, 549 bp, Escherichia coli
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 26 - DNA, 1332 bp, Saccharomyces cerevisiae
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 27 - DNA, 1197 bp, Saccharomyces cerevisiae
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 28 - DNA, 1476 bp, Saccharomyces cerevisiae
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 29 - DNA, 1356 bp, Saccharomyces cerevisiae
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataa

SEQ ID NO: 30 - protein, 4 aa, Saccharomyces cerevisiae
HDEL

SEQ ID NO: 31 - protein, 4 aa, Saccharomyces cerevisiae
DDEL

SEQ ID NO: 32 - protein, 4 aa, Saccharomyces cerevisiae
KDEL

SEQ ID NO: 33 - protein, 29 aa, Saccharomyces cerevisiae
MMSFVSLLLVGILFWATEAEQLTKCEVFQ

SEQ ID NO: 34 - DNA, 33 bp, Artificial Sequence
  Description of Artificial Sequencesynthetic DNA
gccgttgacagagggtccgagctcggtaccaag

SEQ ID NO: 35 - DNA, 34 bp, Artificial Sequence
  Description of Artificial Sequencesynthetic DNA
catactgacccattgtcaatgggtaataactgat

SEQ ID NO: 36 - DNA, 18 bp, Artificial Sequence
  Description of Artificial Sequencesynthetic DNA
tgtccggtaaatggagac

SEQ ID NO: 37 - DNA, 18 bp, Artificial Sequence
  Description of Artificial Sequencesynthetic DNA
tgttctcgctgctcgttt

SEQ ID NO: 38 - DNA, 19 bp, Artificial Sequence
  Description of Artificial Sequencesynthetic DNA
atgggaaagctattacaat

SEQ ID NO: 39 - DNA, 18 bp, Artificial Sequence
  Description of Artificial Sequencesynthetic DNA
caaggttgcaatggccat

SEQ ID NO: 40 - DNA, 19 bp, Artificial Sequence
  Description of Artificial Sequencesynthetic DNA
caatgtagggctatatatg

SEQ ID NO: 41 - DNA, 18 bp, Artificial Sequence
  Description of Artificial Sequencesynthetic DNA
aacttggggaatggcaca

SEQ ID NO: 42 - DNA, 23 bp, Artificial Sequence
  Description of Artificial Sequencesynthetic DNA
tcacgctctgtgtaaagtgtata

SEQ ID NO: 43 - DNA, 30 bp, Artificial Sequence
  Description of Artificial Sequencesynthetic DNA
tgcatctcgagggccgcatcatgtaattag

SEQ ID NO: 44 - DNA, 32 bp, Artificial Sequence
  Description of Artificial Sequencesynthetic DNA
cattagggcccggccgcaaattaaagccttcg

SEQ ID NO: 45 - DNA, 33 bp, Artificial Sequence
  Description of Artificial Sequencesynthetic DNA
cacggagctccagttcgagtttatcattatcaa

SEQ ID NO: 46 - DNA, 35 bp, Artificial Sequence
  Description of Artificial Sequencesynthetic DNA
ctctccgcggtttgtttgtttatgtgtgtttattc

SEQ ID NO: 47 - DNA, 23 bp, Artificial Sequence
  Description of Artificial Sequencesynthetic DNA
atggcttcagaaaaagaaattag

SEQ ID NO: 48 - DNA, 23 bp, Artificial Sequence
  Description of Artificial Sequencesynthetic DNA
ctatttgcttctcttgtaaactt

SEQ ID NO: 49 - DNA, 23 bp, Artificial Sequence
  Description of Artificial Sequencesynthetic DNA
atggaggccaagatagatgagct

SEQ ID NO: 50 - DNA, 23 bp, Artificial Sequence
  Description of Artificial Sequencesynthetic DNA
tcacaattcggataagtggtcta

SEQ ID NO: 51 - DNA, 23 bp, Artificial Sequence
  Description of Artificial Sequencesynthetic DNA
atgccgccgctattcaagggact

SEQ ID NO: 52 - DNA, 23 bp, Artificial Sequence
  Description of Artificial Sequencesynthetic DNA
ttaggatttaatgcaggtgacgg

SEQ ID NO: 53 - DNA, 27 bp, Artificial Sequence
  Description of Artificial Sequencesynthetic DNA
ccaaataaagactccaacactctattt

SEQ ID NO: 54 - DNA, 27 bp, Artificial Sequence
  Description of Artificial Sequencesynthetic DNA
gaattagaagcattattaagtagtgga

SEQ ID NO: 55 - DNA, 27 bp, Artificial Sequence
  Description of Artificial Sequencesynthetic DNA
ggatttaacgcacatgcagctaattta

SEQ ID NO: 56 - DNA, 27 bp, Artificial Sequence
  Description of Artificial Sequencesynthetic DNA
gtctgcttgggttacattttctgaaaa

SEQ ID NO: 57 - DNA, 27 bp, Artificial Sequence
  Description of Artificial Sequencesynthetic DNA
cataccagttatactgcagaccaattg

SEQ ID NO: 58 - DNA, 27 bp, Artificial Sequence
  Description of Artificial Sequencesynthetic DNA
gaatactcattaaagcaaatggtagaa

SEQ ID NO: 59 - DNA, 31 bp, Artificial Sequence
  Description of Artificial Sequencesynthetic DNA
aactgcagatgaccgtttacacagcatccgt

SEQ ID NO: 60 - DNA, 31 bp, Artificial Sequence
  Description of Artificial Sequencesynthetic DNA
cggaattcttattcctttggtagaccagtct

SEQ ID NO: 61 - DNA, 27 bp, Artificial Sequence
  Description of Artificial Sequencesynthetic DNA
tttcagtcccttgaatagcggcggcat

SEQ ID NO: 62 - DNA, 27 bp, Artificial Sequence
  Description of Artificial Sequencesynthetic DNA
cacaaaatcaagattgcccagtatgcc

SEQ ID NO: 63 - DNA, 27 bp, Artificial Sequence
  Description of Artificial Sequencesynthetic DNA
agaagatacggatttcttttctgcttt

SEQ ID NO: 64 - DNA, 27 bp, Artificial Sequence
  Description of Artificial Sequencesynthetic DNA
aactttggtgcaaattgggtcaatgat

SEQ ID NO: 65 - DNA, 27 bp, Artificial Sequence
  Description of Artificial Sequencesynthetic DNA
ttgctctttaaagttttcagaggcatt

SEQ ID NO: 66 - DNA, 27 bp, Artificial Sequence
  Description of Artificial Sequencesynthetic DNA
gcattattaagtagtggaaatacaaaa

SEQ ID NO: 67 - DNA, 27 bp, Artificial Sequence
  Description of Artificial Sequencesynthetic DNA
cctttgtacgctttggagaaaaaatta

SEQ ID NO: 68 - DNA, 27 bp, Artificial Sequence
  Description of Artificial Sequencesynthetic DNA
tctgatcgtttaccatataaaaattat

SEQ ID NO: 69 - DNA, 27 bp, Artificial Sequence
  Description of Artificial Sequencesynthetic DNA
aaggatggtatgacaagaggcccagta

SEQ ID NO: 70 - DNA, 27 bp, Artificial Sequence
  Description of Artificial Sequencesynthetic DNA
tccccgcggatggaggccaagatagat

SEQ ID NO: 71 - DNA, 27 bp, Artificial Sequence
  Description of Artificial Sequencesynthetic DNA
caactcgagtcacaattcggataagtg

SEQ ID NO: 72 - DNA, 38 bp, Artificial Sequence
  Description of Artificial Sequencesynthetic DNA
gctctagagttcgtcgtgtttgcttctcttgtaaactt

SEQ ID NO: 73 - DNA, 32 bp, Artificial Sequence
  Description of Artificial Sequencesynthetic DNA
tatctcgagtcacaattcgtcatgtaaattgg

SEQ ID NO: 74 - DNA, 28 bp, Artificial Sequence
  Description of Artificial Sequencesynthetic DNA
gcagggaccccaattcggataagtggtc

SEQ ID NO: 75 - DNA, 28 bp, Artificial Sequence
  Description of Artificial Sequencesynthetic DNA
gtagggtccctggaggccaagatagatg

SEQ ID NO: 76 - DNA, 29 bp, Artificial Sequence
  Description of Artificial Sequencesynthetic DNA
gcagggaccctttgcttctcttgtaaact

SEQ ID NO: 77 - DNA, 29 bp, Artificial Sequence
  Description of Artificial Sequencesynthetic DNA
gtagggtcctcagaaaaagaaattaggag

SEQ ID NO: 78 - DNA, 18 bp, Artificial Sequence
  Description of Artificial Sequencesynthetic DNA
tgtaaaacgacggccagt

SEQ ID NO: 79 - DNA, 20 bp, Artificial Sequence
  Description of Artificial Sequencesynthetic DNA
taatacgactcactataggg

(19) United States
(12) Patent Application Publication
(10) Pub. No.: US 2004/0063182 A1
(43) Pub. Date: Apr. 1, 2004

(54) Process for producing prenyl alcohol

(76) Inventors: Chikara Ohto, Aichi (JP); Shusei Obata, Aichi (JP)

Correspondence Address: Kenyon & Kenyon, 1500 K Street N W, Suite 700, Washington, DC, 20005-1257, US

(21) Appl. No.: 10/450,941

(22) PCT Filed: Dec. 20, 2001
(86) PCT No.: PCT/JP01/11215

(30) Foreign Application Priority Data
Dec. 28, 2000 (JP) 2000-401701
Dec. 28, 2000 (JP) 2000-406067
Sep. 18, 2001 (JP) 2001-282978

(51) Int. Cl. C12P 7/04

(52) U.S. Cl. 435/157

(57) ABSTRACT

A method of producing a prenyl alcohol(s), comprising culturing a mutant cell that has been mutated so that an amount of squalene synthase gene transcript having translational activity can be reduced, and recovering the prenyl alcohol(s) from the resultant culture.

### TECHNICAL FIELD

The present invention relates to methods of producing prenyl alcohols.

### BACKGROUND ART

The biosynthesis of terpenoids (isoprenoids) begins with the synthesis of geranyl diphosphate (GPP; C₁₀), farnesyl diphosphate (FPP; C₁₅) and geranylgeranyl diphosphate (GGPP; C₂₀), which are straight-chain prenyl diphosphates, through the sequential condensation reactions of isopentenyl diphosphate (IPP; C₅) with an allylic diphosphate substrate (FIG. 1). In FIG. 1, the abbreviations and words in boxes represent enzymes. Specifically, hmgR represents hydroxymethylglutaryl-CoA (HMG-CoA) reductase; GGPS represents GGPP synthase; and FPS represents FPP synthase.

Among prenyl diphosphates, FPP is the most significant biosynthetic intermediate. It is a precursor for the synthesis of tremendous kinds of terpenoids, e.g. steroids including ergosterol (provitamin D₂), the side chains of quinone (vitamin K; VK), sesquiterpenes, squalene (SQ), the anchor molecules of farnesylated proteins, dolichols, bactoprenol, and natural rubber.

GGPP is also a biosynthetic intermediate in vivo, and is essential for the biosynthesis of such compounds as phytoene, lycopene, ficaprenol, retinol (vitamin A; VA), β-carotene (provitamin A), phylloquinone (vitamin K₁; VK₁), tocopherols (vitamin E; VE), the anchor molecules of geranylgeranylated proteins, the side chain of chlorophyll, gibberellins, and the ether lipid of archaea.

It is known that these prenyl diphosphates with up to 20 carbon atoms are condensed into trans forms ((E) forms) and are present as (E,E)-FPP and (E,E,E)-GGPP. Compounds with physiological activities are synthesized from these prenyl diphosphates or prenyl groups with up to 15 or 20 carbon atoms having the all trans (all-E) geometrical isomerism as precursors (K. Ogura and T. Koyama, (1998) Chemical Reviews, 98, 1263-1276; IUPAC-IUB Joint Commission on Biochemical Nomenclature (JCBN) Prenol Nomenclature, Recommendations 1986, (http: //www.chem.qmw.ac.uk/iupac/misc/prenol.htrnl)). Among prenyl diphosphates with up to 20 or 15 carbon atoms, the only one exception that has the cis ((Z) form) geometrical isomerism is neryl diphosphate (C₁₀) known as a precursor for those monoterpenoids as represented by nerol. It has not yet been elucidated whether neryl diphosphate is synthesized through condensation of IPP with dimethylallyl diphosphate (DMAPP; 3,3-dimethylallyl diphosphate) as an allylic diphosphate substrate; or through isomerization of geranyl diphosphate (GPP) that is a trans ((E) form) geometrical isomer with 10 carbon atoms. Those isoprenoids that are synthesized through condensation into cis forms ((Z) forms), e.g. dolichols, bactoprenol (undecaprenol) or natural rubber, are also synthesized from (E,E)-FPP or (E,E,E)-GGPP as an allylic primer substrate (K. Ogura and T. Koyama, (1998) Chemical Reviews, 98, 1263-1276; IUPAC-IUB Joint Commission on Biochemical Nomenclature (JCBN) Prenol Nomenclature, Recommendations 1986, (http: //www.chem.qmw.ac.uk/iupac/misc/prenol.html)).

(E,E)-Farnesol (FOH; C₁₅), which is an alcohol derivative of (E,E)-FPP; (E)-nerolidol (NOH; C₁₅), which is an isomer of the tertiary alcohol thereof; (E,E,E)-geranylgeraniol ((E,E,E)-GGOH; C₂₀), which is an alcohol derivative of (E,E,E)-GGPP; and the like are known as fragrant substances in essential oils used as the ingredient of perfumes. FOH, NOH and (E,E,E)-GGPP are also important as starting materials for the synthesis of various compounds (including the above-mentioned vitamins) useful as pharmacological agents (FIG. 1).

Although it had been believed that all the biosynthesis of IPP is performed via the mevalonate pathway (a pathway in which IPP is synthesized from acetyl-CoA through mevalonate acid), M. Rohmer et al. elucidated a novel IPP biosynthetic pathway using bacteria at the end of 1980's. This is called the non-mevalonate pathway, DXP (1-deoxy-D-xylulose 5-phosphate) pathway, MEP (2-C-methyl-D-erythritol 4-phosphate) pathway or Rohmer pathway, in which IPP is synthesized from glyceraldehyde-3-phosphate and pyruvate through 1-deoxy-D-xylulose 5-phosphate. Thus, two major pathways, i.e. the mevalonate pathway and the non-mevalonate pathway, are known at present as synthetic pathways for IPP.

FOH and NOH are currently produced by chemical synthesis except for small amounts of them prepared from natural products such as essential oils. GGOH is also produced by chemical synthesis (Japanese Unexamined Patent Publication No. 8-13999). Chemically synthesized FOH, NOH or GGOH generally has the same carbon skeleton, but they are obtained as mixtures containing (E) (trans) double bond and (Z) (cis) double bond geometry. (E, E)-FOH, (E)-NOH or (E,E,E)-GGOH, each of which is (all-E) type, is a compound with the geometrical isomerism synthesized in biosynthetic pathways and is industrially valuable. In order to obtain (E, E)-FOH, (E)-NOH or (E,E,E)-GGOH in a pure form, refining by column chromatography, high precision distillation, etc. is necessary. However, it is difficult to carry out high precision distillation of FOH, a thermolable allyl alcohol, or its isomer FOH, or GGOH. Also, the refining of these substances by column chromatography is not suitable in industrial practice since it requires large quantities of solvent and column packings as well as complicated operations of analyzing and recovering serially eluting fractions and removing the solvent; thus, this method is complicated and requires high cost. Actually, the (E, E)-FOH and (E,E,E)-GGOH sold as experimental reagents are very expensive. Under circumstances, it is desired to establish a system for synthesizing not mixtures of cis- and trans-((Z)- and (E)-) isomers but pure products of (E, E)-FOH (hereinafter, just referred to as “FOH”), (E)-NOH (hereinafter, just referred to as “NHO”) and (E,E,E)-GGOH (hereinafter, just referred to as “GGOH”) (i.e., the so-called active prenyl alcohols) in large quantities by controlling the generation of (E)- and (Z)-geometrical isomers or by utilizing the characteristics of repeat structures of reaction products.

The substrates for the biosynthesis of FOH, NOH or GGOH are provided via the mevalonate pathway in cells of, for example, Saccharomyces cerevisiae, a budding yeast. However, even when HMG-CoA reductase that is believed to be a key enzyme for the biosynthesis was used, it has been only discovered that the use increases the accumulation of squalene, a substance commercially available at a greatly low price than FOH and GGOH (Japanese Unexamined Patent Publication No. 5-192184; Donald et al., (i997) Appl. Environ. Microbiol. 63, 3341-3344). Further, it is known that 1.3 mg of FOH per liter of culture broth is accumulated when a squalene synthase gene-deficient clone (ATCC64031) was created by introducing mutations into the squalene synthase gene ERG9 of a particular budding yeast that had acquired sterol intake ability, and cultured (Chambon et al., (1990) Curr. Genet. 18, 4146). The present inventor determined the nucleotide sequence of the squalene synthase gene ERG9 in ATCC64031, and confirmed that this clone has become a squalene synthase gene-deficient clone (erg9 clone) as a result of the introduction of substitution mutations into the coding region of ERG9 and thus acquired the productivity of 1.3 mg/L of FOH. In the coding region of the squalene synthase gene of ATCC64031, the nucleotide at position 745 has been changed from C to T and the nucleotide at position 797 from T to G. As a result, the amino acid residue at position 249 has changed from Gln to termination codon (Q249STOP) and the amino acid residue at position 266 from Ile to Arg (1266R) in the polypeptide encoded by this gene. These changes have led to the expression of a mutant squalene synthase in which the amino acid residue at position 249 and thereafter are deleted, and which has no enzyme activity. However, when ERG9 is made deficient in a conventional strain, the deficiency is lethal to the strain because the strain cannot synthesize ergosterol essential for growth and it has no function to intake sterols from the outside under conventional culture conditions. Thus, an ERG9-deficient clone cannot be obtained; it is impossible to construct an FOH production system. Furthermore, no method of biosynthesis of (E)-NOH (hereinafter, referred to as “NOH”) is known. Even if it is possible to provide to a ERG9-deficient clone with a novel character that avoids the lethality, the cultivation of such a clone will require disadvantageous conditions to industrial production, e.g., necessity to add ergosterol to the medium.

With respect to the biosynthesis of GGOH, production of 0.66-3.25 mg per liter of culture broth is achieved by culturing plant cells in Japanese Unexamined Patent Publication No. 9-238692. However, this method needs an expensive plant cell culture medium inappropriate for industrial application and also requires light for culturing cells. Thus, this method is not practical even when compared to the conventional GGOH preparation from natural products such as essential oils. There is known no method of biosynthesis of GGOH suitable for industrialization, e.g. biosynthesis by microorganisms.

### DISCLOSURE OF THE INVENTION

It is an object of the present invention to provide a method of producing prenyl alcohols by reducing the amount of squalene synthase gene transcript having translational activity.

As a result of intensive and extensive researches toward the solution of the above problem, the present inventors have developed a prenyl alcohol production system by culturing a mutant cell in which the transcription promoter region of squalene synthase gene has been replaced with a transcription repression type promoter under transcription repression conditions so that an amount of squalene synthase gene transcript having translational activity can be reduced. Further, in order to establish a system to artificially express an IPP biosynthetic pathway-related enzyme gene (represented by HMG-CoA reductase gene), a mutant thereof or a fusion gene thereof in a host, expression shuttle vectors were constructed which comprise a constitutive or inducible transcription promoter and various auxotrophic markers. A gene of interest or a mutant thereof was inserted into the shuttle vector, which was then introduced into the mutant cell of the invention where an amount of squalene synthase gene transcript having translational activity can be reduced. From a culture of the resultant cell, a prenyl alcohol(s) could be obtained, and the above object could be achieved. Thus, the present invention has been completed.

The present invention relates to the following inventions.

(1) A method of producing a prenyl alcohol(s), comprising culturing a mutant cell that has been mutated so that an amount of squalene synthase gene transcript having translational activity can be reduced, and recovering the prenyl alcohol(s) from the resultant culture.

(2) A method of producing a prenyl alcohol(s), comprising preparing a recombinant by introducing a recombinant DNA for expression or a DNA for genomic integration each comprising an IPP biosynthetic pathway-related enzyme gene into a mutant cell that has been mutated so that an amount of squalene synthase gene transcript having translational activity can be reduced, culturing the resultant recombinant, and recovering the prenyl alcohol(s) from the resultant culture.

(3) A method of producing a prenyl alcohol(s), comprising culturing a mutant cell in which the transcription promoter region of squalene synthase gene has been replaced with a transcription repression-type promoter under transcription repression conditions; reducing an amount of squalene synthase gene transcript having translational activity; and recovering the prenyl alcohol(s) from the resultant culture.

(4) A method of producing a prenyl alcohol(s), comprising preparing a recombinant by introducing a recombinant DNA for expression or a DNA for genomic integration each comprising an IPP biosynthetic pathway-related enzyme gene into a mutant cell in which the transcription promoter region of its squalene synthase gene has been replaced with a transcription repression-type promoter so that an amount of squalene synthase gene transcript having translational activity can be reduced, culturing the recombinant under transcription repression conditions; and recovering the prenyl alcohol(s) from the resultant culture.

In the methods described above, the IPP biosynthetic pathway-related enzyme gene includes enzyme genes involved in those reactions whrere IPP is synthesized from compounds located in the glycolytic pathway or citrate cycle, as well as various prenyl diphosphate synthase genes. The IPP biosynthetic pathway-related enzyme gene includes not only those enzyme genes involved in the mevalonate pathway but also those enzyme genes involved in the so-called non-mevalonate pathway [also called DXP (1−deoxy-D-xylulose 5-phosphate) pathway, MEP (2-C-methyl-D-erythritol 4-phosphate) pathway or Rohmer pathway] that is a novel IPP biosynthetic pathway recently found in prokaryotes and chloroplasts.

In the methods described above, specific examples of the transcription repression type promoter include GAL1 promoter. Specific examples of the transcription repression conditions for GAL1 promoter include the use of a glucose-containing medium. As the IPP biosynthetic pathway-related enzyme gene, any one selected from the group consisting of the following genes (a) through (l) may be given.

(a) farnesyl diphosphate synthase gene

(b) geranylgeranyl diphosphate synthase gene

(c) hydroxymethylglutaryl-CoA reductase gene

(d) isopentenyl diphosphate Δ-isomerase gene

(e) mevalonate kinase gene

(f) acetyl-CoA acetyltransferase gene

(g) hydroxymethylglutaryl-CoA synthase gene

(h) phosphomevalonate kinase gene

(i) diphosphomevalonate decarboxylase gene

(j) a mutant gene of any one of the above genes (a) through (i)

(k) a fusion gene composed of a gene selected from the group consisting of the above genes (a) through (i) or a mutant thereof, and another gene or a mutant gene thereof (an artificial sequence may be inserted into the junction site between the two genes)

(l) a gene obtained by introducing an addition, substitution or insertion mutation into any one of the above genes (a) through (k) so that the polypeptide encoded by the resultant gene contains an endoplasmic reticulum signal.

In the junction site of the above-described fusion gene, a nucleotide sequence encoding an artificial amino acid sequence may be inserted freely so that the polypeptides encoded by the two genes before fusion can take conformations that allow them to function appropriately. For example, a sequence 5′ GGGTCC 3′ encoding Gly Ser may be inserted.

As endoplasmic reticulum signals, His Asp Glu Leu (SEQ ID NO: 30; called “HDEL sequence”) or Asp Asp Glu Leu (SEQ ID NO: 31) located at the C-terminal is known in Saccharomyces cerevisiae, and Lys Asp Glu Leu (SEQ ID NO: 32) located at the C-terminal is known in eukaryotes in general as a C-terminal signal. It is known that they work as an endoplasmic reticulum retention signal completely equivalent to them in function (B. Lewin, Genes V, (1994), Oxford University Press, New York, U.S.A., pp. 279-318; B. Alberts et al., Molecular Biology of The Cell, third edition (1994), Garland Publishing. Inc., New York, U.S.A., §12-§13) (hereinafter, these sequences are generically terned “HDEL sequence or the like”). In addition to these endoplasmic reticulum retention signals consisting of the 4 amino acid residues located at the C-terminal, other signal peptides from a part of the domains of ER transition proteins, e.g., the signal peptide ⁺H₃N Met Met Ser Phe Val Ser Leu Leu Leu Val Gly Ile Leu Phe Trp Ala Thr Glu Ala Glu Gln Leu Thr Lys Cys Glu Val Phe Gln (SEQ ID NO: 33) located at the N-terminal that functions as a transport signal to ER may also be used (B. Lewin, Genes V(1994), Oxford University Press, New York, U.S.A., pp. 279-318; B. Alberts et al., Molecular Biology of The Cell, third edition (1994), Garland Publishing. Inc., New York, U.S.A., §12-§13).

A polypeptide in which an endoplasmic reticulum signal has been added, substituted or inserted by introducing an addition, substitution or insertion mutation into its gene may also be regarded as a mutant polypeptide in which 4 amino acid residues are added, substituted or inserted in the corresponding wild type polypeptide. A gene encoding such a mutant polypeptide may also be regarded as a mutant gene in which a nucleotide sequence of about 1-12 nucleotides is added, substituted or inserted in the corresponding wild type gene. Also, a polypeptide encoded by the above-described fusion gene may be regarded as a mutant polypeptide in which more than 300 amino acid residues are added, substituted or inserted in a wild type polypeptide. The fusion gene may also be regarded as a mutant gene in which a nucleotide sequence of about 1000 nucleotides is added, substituted or inserted in a wild type gene. That is, a gene or fusion gene encoding an amino acid sequence in which a signal has been newly created is in itself a mutant gene.

In order to create an HDEL sequence or the like at the C-terminal, insertion of one base that would cause a frame shift resulting in the creation of an HDEL sequence or the like may also be used. It is also possible to replace 2 nucleotides to thereby create an HDEL sequence or the like from the original C-terminal sequence (e.g., HDGI). If an HDEL sequence or the like is present near the C-terminal (e.g., in the case of BTS1), an HDEL sequence or the like can be created at the C-terminal by simply introducing a stop codon. Alternatively, it is possible to newly add 12 nucleotides to the C-terminal to thereby create an HDEL sequence or the like at the C-terminal.

As a host cell to be mutated, yeast, yeast belonging to Ascomycota, yeast belonging to Basidiomycota, or yeast belonging to Fungi Imperfecti may be used, for example. Preferably, yeast belonging to Ascomycota, in particular, budding yeast such as Saccharomyces cerevisiae, Candida utilis or Pichia pastris; and fission yeast such as Shizosaccharomyces pombe may be used. Not only species but also strains are not limited as long as they can produce prenyl alcohol. In the case of S. cerevisiae, specific examples of useful strains include A451, YPH499, YPH500, W303-1A and W303-1B. Specific examples of the prenyl alcohol include FOH, NOH and/or GGOH.

In the methods described above, the mutant cell or the recombinant may be cultured under non-transcription repression conditions prior to the cultivation under transcription repression conditions. The cultivation under transcription repression conditions means cultivation in a glucose-containing medium. The cultivation under non-transcription repression conditions means cultivation in a galactose-containing medium.

As the farnesyl diphosphate synthase gene, a gene encoding the amino acid sequence as shown in SEQ ID NO: 2 or 4 may be used. As the geranylgeranyl diphosphate synthase gene, a gene encoding the amino acid sequence as shown in SEQ ID NO: 6 may be used. As the hydroxymethylglutaryl-CoA reductase gene, a gene encoding the amino acid sequence as shown in SEQ ID NO: 8 may be used. As the diphosphomevalonate decarboxylase gene, a gene encoding the amino acid sequence as shown in SEQ ID NO: 10 may be used. As the mutant of hydroxymethylglutaryl-CoA reductase gene, a mutant comprising the nucleotide sequence as shown in any one of SEQ ID NOS: 11, 13 and 15 through 24 may be used.

(5) A mutant cell that has been mutated so that an amount of squalene synthase gene transcript having translational activity can be reduced.

(6) A mutant cell in which the transcription promoter region of its squalene synthase gene has been replaced with a transcription repression-type promoter so that an amount of squalene synthase gene transcript having translational activity can be reduced.

Hereinbelow, the present invention will be described in detail. The present specification encompasses the contents described in the specifications and/or drawings of Japanese Patent Application Nos. 2000-401701, 2000403067 and 2000-282978 based on which the present application claims priority.

The inventors have attempted to develop a system for producing an active-type prenyl alcohol, in particular, FOH, NOH or GGOH in vivo by using metabolic engineering techniques.

In the present invention, yeast, in particular, a budding yeast Saccharomyces cerevisiae was selected as a host cell. In the host cell, a part of the genomic DNA that is believed to be the transcription promoter region of its squalene synthase gene ERG9 was replaced with the transcription promoter of GAL1 gene, which is one of transcription repression type promoters capable of repression with glucose, to thereby prepare strains designated EUG. Thus, a system has been developed which allows accumulation of a prenyl alcohol(s) in culture broth by simply culturing a conventional recombinant host strain (that has no sterol intake ability under aerobic culture conditions) aerobically in a conventional glucose-containing medium without special additives (e.g., YM7 medium, YPD medium, or SD medium).

Generally, FPP is synthesized by the catalytic action of FPP synthase using IPP or DMAPP as a substrate. After the synthesis of FPP, usually, reactions do not proceed toward dephosphorylation to FOH, but proceed toward the synthesis of squalene and various sterols by squalene synthase; the synthesis of GGPP and prenylated proteins by GGPP synthase; the synthesis of medium or long chain prenyl diphosphates (such as hexaprenyl diphosphate, heptaprenyl diphosphate), ubiquinone and prenylated proteins by medium chain prenyl diphosphate synthases such as hexaprenyl diphosphate synthase or heptaprenyl diphosphate; and so on (FIG. 1) (K. Ogura and T. Koyama, (1998) Chemical Reviews, 98: 1263-1276). This means that when squalene synthase activity in a cell is lowered by reducing an amount of squalene synthase gene transcript having translational activity, the following can be expected to occur: accumulation of FPP to some extent; accumulation of prenylated proteins and ubiquinone, which are final products of the synthetic pathway using FPP as a substrate; or inhibition of cell growth because the synthesis of ergosterol, an essential component for the growth of the cell, becomes impossible. However, it is completely unpredictable that such reduction of squalene synthase activity makes it possible to construct an FOH production system with high productivity of more than 10 mg/L or more than 100 mg/L. It is also unpredictable that a production system for NOH or GGOH (dephosphorylated product of GGPP) can be obtained by such reduction (FIG. 1). Furthermore, even if IPP synthesis is enhanced by increasing the enzyme activity of an IPP biosynthetic pathway-related enzyme, it is unpredictable whether the enhanced enzyme activity would increase FPP synthesis or GGPP synthesis, and it is totally unpredictable that such enhance enzyme activity would lead to high production of FOH and GGOH (dephosphorylated products) and NOH (an isomer of FOH) (FIG. 1).

The present invention has found that it is possible to allow a cell to produce prenyl alcohols such as FOH, NOH and GGOH that are not explicitly mentioned even in the commonly accepted metabolic pathway map (Gerhard Michael (ed.), Biochemical Pathways: An Atlas of Biochemistry and Molecular Biology (1999), John Wiley & Sons, Inc., New York) by mutating the cell so that an amount of squalene synthase gene transcript having translational activity can be reduced and culturing the mutant cell under specific conditions to thereby reduce the transcript. Thus, a biological mass production system for prenyl alcohol has been developed.

The present invention has also developed another biological mass production system for prenyl alcohol by introducing an IPP biosynthetic pathway-related enzyme gene, a mutant thereof or a fusion gene thereof as a transcription unit into the mutant cell of the above-described prenyl alcohol production system of the invention.

1. Preparation of Squalene Synthase Gene Transcript-Mutated Strains

The mutant cell used in the invention comprises the squalene synthase gene of the host cell that has no substitution, insertion or addition in the coding region but has been modified so that the amount of its transcript having translational activity can be reduced. In order to reduce the amount of transcript having translational activity, several approaches may be taken. For example, a method of repressing the transcriptional activity of a gene of interest to thereby reduce the amount of its transcript per se may be used. Alternatively, a method of repressing the translational activity of transcribed mRNA using antisense RNA may be used. Transcriptional activity may be repressed, for example, by replacing the transcription promoter region of the squalene synthase gene of the host with a transcription repression type promoter, or by creating a nucleotide sequence having transcription repressive activity in a region involved in the transcription of the squalene synthase gene. Translational activity may be repressed, for example, by over-expressing a gene from which an antisense RNA against the squalene synthase gene is transcribed. Antisense RNA means an RNA having a nucleotide sequence complementary to the RNA transcribed from a target gene. Antisense RNA reduces the amount of transcript having translational activity in the transcription/translation system in both prokaryotic cells and eukaryotic cells; it has a function as a repressor of gene expression. For example, it is reported that the synthesis of OmpF protein can be repressed by reducing the amount of mRNA from E coli ompF gene with transcriptional activity using antisense RNA [Takeshi Mizuno in Expression and Control of Genes I (Series: Advances in Molecular Biology), The Molecular Biology Society of Japan (Eds.), published by Maruzen Co. on Jun. 30, 1989, ISBN4-621-03375-1, Chapter 5 “Control of Bacterial Membrane Protein Synthesis”]. As Mizuno described in this reference, antisense RNAs against certain genes occur in nature, and if a target gene has been decided, it is easy for one of ordinary skill in the art to create an artificial antisense RNA having expression repressive activity by genetic engineering techniques. The artificial antisense RNA technology has been applied to various hosts, including not only the bacterium E. coli but also higher organisms, e.g., mammals (mouse), insects (Drosophila melanogaster) and angiosperms (tomato) [Mizuno et al., (1984) Proc. Natl. Acad. Sci. U.S.A., 81, 1966-1970; Aiba et al, (1987) J Bacteriol, 169, 3007-3012; Green et al., (1986) Annu. Rev. Biochem., 55, 569-597; Harland et al, (1985) J. Cell. Biol., 101, 1094-1099; Coleman et al., (1984) Cell, 37, 429-36; Han et al., (1991) Proc. Natl. Acad. Sci. U.S.A., 88, 4313-4317; Hackett et al., (2000) Plant Physiol., 124, 1079-86)].

As the host cell used in the invention, such a cell may be selected which has been widely used in the fermentation industry from old times, has the mevalonate pathway as an IPP biosynthetic pathway, and can be subjected to various genetic engineering techniques. For example, yeast such as budding yeast may be given. Specific examples of budding yeast include Saccharomyces cerevisiae. However, the host is not necessarily limited to yeast such as S. cerevisiae. Various other cells may also be used as a host as long as they possess a squalene synthase gene. By reducing an amount of squalene synthase gene transcript having translational activity in the host cell, it is possible to allow the cell to produce various active prenyl alcohols even if any deficiency has not been introduced into the squalene synthase per se by introducing a substitution, insertion or deletion mutation(s) into the coding region of the gene.

In order to reduce the amount of squalene synthase gene transcript having translational activity, a transcription repression type promoter may be used, for example. As a transcription repression type promoter, any transcription promoter may be used as long as it can control transcription under various culture conditions. Specific examples of useful transcription promoters include promoters responsible for the transcription of GAL1, GAL2, GAL7, GAL10, GAL80 and MEL1 whose transcription is repressed in a glucose containing medium; ADH1 and ENO2 whose transcription is repressed under glucose-deprived conditions; PHO5 and PHO81 whose transcription is repressed under high phosphate conditions; and HO gene whose transcription is repressed in phases other than G₁ in cell cycle. In addition, a promoter that is responsible for the transcription of a gene whose transcript is detected considerably under specific conditions but detected little under other conditions, such as reported in L. Wodicka et al., (1997) Nature Biotechnology, 15, 1359-1367, may also be used as a transcription repression type promoter. For example, (i) promoters responsible for the transcription of such genes as HSP12, INO1, YBR147W and YGR243W whose transcript is not detected in a conventional nutrient medium (rich medium) but detected considerably in minimal medium; or (ii) promoters responsible for the transcription of such genes as YDRO46C, GNP1, CHA1 and PTR2 whose transcript is not detected in minimal medium but detected considerably in a conventional nutrient medium, may be enumerated. Further, it is also possible to use a transcription repression type promoter of an organism other than budding yeast. The promoter does not necessarily have transcription repressive activity in the host cell. For example, a transcription promoter may be used which acquires transcription repressive activity only after the introduction of a transcription repression factor gene into the host cell. Further, it is not necessarily required to use a transcription promoter capable of direct repression of transcription (i.e., control of mRNA synthesis) in the host cell, as along as squalene gene transcript having translational activity can be reduced. By deactivating the translational activity of mRNA (the transcript) by antisense RNA technology or the like, it is possible to achieve the same effect as achieved by the use of a transcription repression type promoter, i.e., to allow the host cell to produce prenyl alcohol without modifying the coding region of its squalene synthase gene.

Hereinbelow, a method of replacing the transcription promoter of a squalene synthase gene with transcription repression type promoter will be described using Saccharomyces cerevisiae as a hose cell.

First, a gene map around squalene synthase gene ERG9 was obtained from a yeast genome database (Saccharomyces Genome Database (SGD); http://genome-www.stanford.edu./Saccharomyces/). Then, PCR primers for amplifying DNA fragments for replacing ERG9 transcription promoter (ERG9p) were designed based on the map (FIG. 2).

Subsequently, pYES2 (FIG. 2C and FIG. 3) were digested with restriction enzymes and blunt-ended with Klenow enzyme, followed by self-ligation to yield pYES2Δ without 2 μori (FIG. 2B). Using pYES2Δ as a template, a DNA fragment comprising a transformant selection marker gene URA3 and a transcription promoter GAL1p was amplified by PCR. A partial DNA sequence of ERG9 gene was ligated to one end of the resultant DNA fragment, and a partial DNA sequence of YHR189W located in the proximity of ERG9 gene was ligated to the other end of this DNA fragment. Thus, a DNA fragment for transformation was prepared (FIG. 2A).

In the present invention, a host for preparing a mutant cell is not particularly limited. Yeast that is important from the viewpoint of industrial application may be used as a host. For example, yeast belonging to Ascomycota, yeast belonging to Basidiomycota, or yeast belonging to Fungi Imperfecti may be used. Preferably, yeast belonging to Ascomycota, in particular, budding yeast such as Saccharomyces cerevisiae, Candida utilis or Pichia pastris; and fission yeast such as Shizosaccharomyces pombe may be used. In the case of S. cerevisiae, specific examples of useful strains include A451, YPH499, YPH500, W303-1A and W303-1B described below.

A451 (ATCC200589; MATα can1 leu2 trp1 ura3 aro7)

YPH499 (ATCC76625; AMTα ura3-52 lys2-801 ade2-101 trp1-Δ63 his3-Δ200 leu2-Δ1; Stratagene)

YPH500 (ATCC76626; MATα ura3-52 lys2-801 ade2-101 trp1-Δ63 his3-Δ200 leu2-Δ1; Stratagene)

W303-1A (ATCC208352; MATα leu2-3 leu2-112 his3-11 ade2-1 ura3-1 trp1-1 can1-100)

W303-1B (ATCC208353; MATα leu2-3 leu2-112 his3-11 ade2-1 ura3-1 trp1-1 can1-100)

The transformation of a host cell with the above DNA fragment may be performed by any of the conventional technique or with a commercial kit. For example, a vector comprising the DNA fragment is introduced into the host yeast using Frozen EZ Yeast Transformation II Kit purchased from Zymo Research (Orange, Calif.).

The DNA fragment thus introduced into the host is integrated into the genome of the host by homologous recombination. As a result, a mutant cell is obtained in which the transcription promoter region of its squalene synthase gene ERG9 is replaced with GAL1 promoter that is a transcription repression type promoter.

The recombinants obtained by replacing ERG9 promoter with GAL1 promoter (a transcription repression type promoter) in order to reduce the amount of squalene synthase gene ERG9 transcript having translational activity were designated EUG (ERG9p::URA3-GAL1p) strains. A451-derived clones are designated EUG1 through EUG10. YPH499-derived clones are designated EUG11 through EUG20. YPH500-derived clones are designated EUG21 through EUG30. W303-1A-derived clones are designated EUG31 through EUG50. W303-1B-derived clones are designated EUG51 through EUG70.

EUG1 through EUG10 (A451; ERG9p::URA3-GAL1p) are A451-derived strains established in the present invention and are capable of reducing the arnount of squalene synthase gene ERG9 transcript having translational activity. These strains comprise the polypeptide-coding region of squalene synthase ERG9, a transformant selection marker gene URA3, and a transcription promoter GAL1p located in the ERG9 locus.

EUG11 through EUG20 (YPH499; ERG9p::URA3-GAL1p) are YPH499-derived strains established in the present invention and are capable of reducing the amount of squalene synthase gene ERG9 transcript having translational activity. These strains comprise the polypeptide-coding region of ERG9, the URA3, and the GAL1p located in the ERG9 locus.

EUG21 through EUG30 (YPH500; ERG9p::URA3-GAL1p) are YPH500-derived strains established in the present invention and are capable of reducing the amount of squalene synthase gene ERG9 transcript having translational activity. These strains comprise the polypeptide-coding region of ERG9, the URA3, and the GAL1p located in the ERG9 locus.

EUG31 through EUG50 (W303-1A; ERG9p::URA3-GAL1p) are W303-1A-derived strains established in the present invention and are capable of reducing the amount of squalene synthase gene ERG9 transcript having translational activity. These strains comprise the polypeptide-coding region of ERG9, the URA3, and the GAL1p located in the ERG9 locus.

EUG51 through EUG70 (W303-1B; ERG9p::URA3-GAL1p) are W303-1B-derived strains established in the present invention and are capable of reducing the amount of squalene synthase gene ERG9 transcript having translational activity. These strains comprise the polypeptide-coding region of ERG9, the URA3, and the GAL1p located in the ERG9 locus.

In the present invention, various prenyl alcohols can be obtained by culturing the thus obtained EUG strains in a glucose-containing medium (i.e., transcription repression condition for GAL1p) to thereby reduce an amount of squalene synthase gene transcript having transcriptional activity (cultivation method will be described later). According to the present invention, prenyl alcohols can be obtained without the introduction of a substitution, insertion or deletion mutation(s) into the coding region of a squalene synthase gene as known in ATCC64031, and without the addition of special additives such as ergosterol and surfactants to the medium.

2. Preparation of Recombinant DNAs for Expression or DNA Fragments for Genomic Integration

In the present invention, the above-described EUG strains as they are may be cultured. Alternatively, these strains may be cultured after further transformation by introducing thereinto a recombinant DNA for expression or a DAN fragment for genomic integration each comprising an IPP biosynthetic pathway-related enzyme gene. The recombinant DNA for expression used in the further transformation of EUG strains can be obtained by ligating or inserting a transcription promoter DNA and a transcription terminator DNA into an IPP biosynthetic pathway-related enzyme gene. It is also possible to prepare in advance a gene expression cassette (transcription unit) comprising an IPP biosynthetic pathway-related enzyme gene to which a transcription promoter and a transcription terminator have been ligated, and to incorporate the cassette into a vector. The ligation or insertion of the promoter and terminator may be performed in any order. However, it is preferable to ligate a promoter upstream of an IPP biosynthetic pathway-related gene and to ligate a terminator downstream of the gene. Alternatively, in the present invention, an IPP biosynthetic pathway-related gene, a transcription promoter and a transcription terminator may be incorporated into an appropriate DNA, e.g. a vector, in this order. If the direction of transcription is properly considered, the incorporation may be performed in any order. An enhancer sequence to enhance the gene expression and various cis elements to control the gene expression may also be incorporated around the transcription unit.

Specific examples of IPP biosynthetic pathway-related genes include the following genes.

(a) farnesyl diphosphate synthase gene (FPP synthase gene)

(b) geranylgeranyl diphosphate synthase gene (GGPP synthase gene)

(c) hydroxymethylglutaryl-CoA reductase gene (HMG-CoA reductase gene)

(d) isopentenyl diphosphate Δ-isomerase gene (IPP Δ-isomerase gene)

(e) mevalonate kinase gene

(f) acetyl-CoA acetyltransferase gene

(g) hydroxymethylglutaryl-CoA synthase gene

(h) phosphomevalonate kinase gene

(i) diphosphomevalonate decarboxylase gene

Specific examples of FPP synthase gene include Saccharomyces cerevisiae-derived ERG20 (SEQ ID NO: 1), Escherichia coli-derived ispA (SEQ ID NO: 3), Bacillus stearothermophilus-derived FPP synthase genes (Japanese Unexamined Patent Publication No. 5-219961; U.S. Pat. No. 5,786,192) and Synechococcus elongatus-derived FPP synthase gene (C. Ohto et al., (1999) Plant Mol. Biol., 40, 307-321). Specific examples of GGPP synthase gene include Saccharomyces cerevisiae-derived BTS1 (SEQ ID NO: 5), Sulfolobus acidocaldarius-derived crtE (C. Ohto et al., (1998) Biosci. Biotechnol. Biochem., 62, 1243-1246; Japanese Unexamined Patent Publication No. 7-308913; U.S. Pat. No. 5,773,273), Thermus thermophilus-derived Tth GGPP synthase gene (C. Ohto et al., (1999) Biosci. Biotechnol. Biochem., 63, 261-270; Japanese Unexamined Patent Publication No.9-107974, U.S. Pat. No. 6,107,072), and Synechococcus elongatus-derived GGPP synthase gene (C. Ohto et al., (1999) Plant Mol. Biol., 40, 307-321). Specific examples of HMG-CoA reductase gene include Saccharomyces cerevisiae-derived HMG1 (SEQ ID NO: 7) and HMG2, and Streptmyces sp. CL190-derived HMG-CoA reductase gene (S. Takahashi et al., (1999) J. Bacteriol., 181, 1256-1263). Specific examples of IPP Δ-isomerase gene include Escherichia coli-derived idi (SEQ ID NO: 25). Further, IPP biosynthetic pathway-related genes having the following nucleotide sequences, respectively, may be enumerated.

Mevalonate kinase gene ERG12 (SEQ ID NO: 26)

Acetyl-CoA acetyltransferase gene ERG10 (SEQ ID NO: 27)

HMG-CoA synthase gene HMGS (ERG13) (SEQ ID NO: 28)

Phosphomevalonate kinase gene ERG8 (SEQ ID NO: 29)

Diphosphomevalonate decarboxylase gene ERG19 (SEQ ID NO: 7)

Depending on the host, its major IPP biosynthetic pathway is the non-mevalonate pathway. Therefore, non-mevalonate pathway-related enzyme genes such as DXP synthase gene, DXP reductoisomerase, MEP cytidyltransferase, CDP-ME kinase gene and MECDP synthase gene (K. Kaneda et al., (2001) PNAS, 98, 932-937; T. Kuzuyama et al., (2000) J. Bacteriol., 182, 891-897; T. Kuzuyama et al., (2000) J. Biol. Chem., 275, 19928-19932; S. Takahashi et al., (1998) PNAS, 95, 9879-9884) may be used for such a host as an IPP biosynthetic pathway-related gene.

One of ordinary skill in the art can obtain these genes easily by a known method of gene isolation or with a commercial kit for gene isolation.

In the present invention, mutants (sometimes referred to as “mutant type gene(s)”) of the above-described IPP biosynthetic pathway-related genes may also be used.

Mutants of the above-described IPP biosynthetic pathway-related genes may be deletion mutants having a deletion of a specific region (e.g., an HMG-CoA reductase gene with a deletion of 2217 nucleotides at the maximum), or mutants having a deletion, addition, substitution or insertion of one or several to ten nucleotides in the nucleotide sequence of a wild type gene or the above-mentioned deletion mutant. Further, about 1000 nucleotides may be added, substituted or inserted. Accordingly, the amino acid sequence encoded by such a mutant may have a mutation(s). Specifically, the amino acid sequence of an IPP biosynthetic pathway-related enzyme (e.g., SEQ ID NO: 2 for FPP synthase; SEQ ID NO: 6 for GGPP synthase; or SEQ ID NO: 8 for wild type HMG-CoA reductase) may have a mutation(s) such as deletion, addition, substitution or insertion of one or several (e.g., one to ten, preferably, one to three) amino acids or more than 300 amino acids. The amino acid sequence of a wild type HMG-CoA reductase (SEQ ID NO: 8) may have a deletion of 739 amino acids at the maximum, and such a deletion mutant type enzyme may further have a mutation(s) such as deletion, addition, substitution or insertion of one or several (e.g., one to ten, preferably, one to three) amino acids. Specific examples of such mutant enzymes include a mutant enzyme FHDEL obtained by adding 4 amino acids to a wild type FPP synthase; mutant enzymes FGG and FGGHDEL obtained by adding more than 300 amino acids to the C-terminal of a wild type FPP synthase; a mutant enzyme GGF obtained by adding more than 300 amino acids to the N-terminal of a wild type FPP synthase; a mutant enzyme GGFHDEL obtained by adding more than 300 amino acids and 4 amino acids to the N-terminal and the C-terminal of a wild type FPP synthase, respectively; mutant enzymes GGF and GGFHDEL obtained by adding more than 300 amino acids to the C-terminal of a wild type GGPP synthase; a mutant enzyme FGG obtained by adding more than 300 amino acids to the N-terminal of a wild type GGPP synthase; and a mutant enzyme FGGHDEL obtained by adding more than 300 amino acids to the N-terminal of a wild type GGPP synthase and deleting 10 amino acids at the C-terminal of the synthase.

Specific examples of mutant genes useful in the invention include a mutant type ERG20 in pRS435FHDEL obtained by adding 12 nucleotides to the 3′ end of ERG20; a mutant type ERG20 in pRS435FGG obtained by adding a nucleotide sequence of about 1000 nucleotides encoding BTS1 and Gly Ala to the 3′ end of ERG20; a mutant type ERG20 in pRS435FGGHDEL obtained by deleting 30 nucleotides at the 3′ end of the preceding mutant type ERG20; a mutant type ERG20 in pRS435GGF obtained by adding a nucleotide sequence of about 1000 nucleotides encoding BTS1 and Gly Ala to the 5′ end of ERG20; a mutant type ERG20 in pRS435GGFHDEL obtained by adding a nucleotide sequence of about 1000 nucleotides encoding BTS1 and Gly Ala to the 5′ end of ERG20 and adding 12 nucleotides to the 3′ end of ERG20; a mutant type BTS1 in pRS435GGHDEL obtained by deleting 30 nucleotides at the 3′ end of BTS1; a mutant type BTS1 in pRS435GGF obtained by adding a nucleotide sequence of about 1000 nucleotides encoding ERG20 and Gly Ala to the 3′ end of BTS1; a mutant type BTS1 in pRS435GGFHDEL obtained by adding 12 nucleotides to the preceding mutant type BTS1; a mutant type BTS1 in pRS435FGG obtained by adding a nucleotide sequence of about 1000 nucleotides encoding ERG20 and Gly Ala to the 5′ end of BTS1; and a mutant type BTS1 in pRS435FGGHDEL obtained by adding a nucleotide sequence of about 1000 nucleotides encoding ERG20 and Gly Ala to the 5′ end of BTS1 and deleting 30 nucleotides at the 3′ end of BTS1.

In the PCR (polymerase chain reaction) amplification of IPP biosynthetic pathway-related genes, substitution mutations of nucleotides that occur in DNA fragments obtained by amplifying wild-type DNA using a DNA polymerase of low fidelity (such as Taq DNA polymerase) are called “PCR errors”. For example, an HMG-CoA reductase gene that has a nucleotide sequence having nucleotide substitutions (which may cause substitution mutations in the encoded polypeptide) attributable to PCR errors when a wild-type HMG-CoA reductase gene (SEQ ID NO: 7) was used as a template may also be used in the present invention. This HMG-CoA reductase gene is designated “HMG1′”. An embodiment of nucleotide substitutions resulting from PCR errors when the wild-type HMG-CoA reductase gene (SEQ ID NO: 7) was used as a template is shown in FIG. 4A. HMG1′ has the nucleotide sequence as shown in SEQ ID NO: 11, and the amino acid sequence encoded thereby is shown in SEQ ID NO: 12. In FIG. 4A, mutations of nucleotides are expressed in the following order: the relevant nucleotide before substitution (in one letter abbreviation), the position of this nucleotide when the first nucleotide in the initiation codon of the HMG-CoA reductase gene is taken as 1, and the nucleotide after substitution (in one letter abbreviation). The mutations of amino acids contained in the amino acid sequence of the PCR error-type HMG-CoA reductase are expressed in the following order: the relevant amino acid before substitution (in one letter abbreviation), the position of this amino acid in the HMG-CoA reductase, and the amino acid after substitution (in one letter abbreviation). Further, the PCR error-type nucleotide sequence described above may be modified partially by techniques such as site-directed mutagenesis. Such a modified HMG-CoA reductase gene (SEQ ID NO: 14) encoding a modified HMG-CoA reductase (SEQ ID NO: 13) may also be used in the present invention.

As examples of HMG-CoA reductase genes (including PCR error-type) encoding deletion mutants in which predicted transmembrane domains of HMG-CoA reductase gene are deleted, HMG1Δ genes are shown in FIG. 4B that are deletion mutants of the PCR error-type HMG-CoA reductase gene HMG1′. The upper most row represents HMG1′ gene without deletion. The portion indicated with thin solid line (-) is the deleted region. Table 1 below shows which region of HMG1′ gene (SEQ ID NO: 11) has been deleted for each of the deletion mutants. Deletion mutants of HMG1′ are expressed as “HMG1Δxxy” according to the deletion pattern, in which “xx” represents the deletion pattern and “y” represents a working number (any figure). In FIG. 4B, “Δ026” is shown as one example of HMG1Δ02y. (Likewise, examples of other deletion patterns are also shown.) Deletion of 1317 nucleotides is the maximum deletion.

TABLE 1
Embodiment of Deletions

| | | | | Deletion of | | Sequence |
|---|---|---|---|---|---|---|
| Designation of | | | | Predicted | Deleted | after |
| Deletion Mutant | Primer 1 | Primer 2 | Plasmid | Transmembrane | Region | Deletion |
| HMG1 Δ 02y | HMG1 (558-532) | HMG1 (799-825) | pYHMG02X | #2-#3 | 559˜798 | SEQ ID NO:15 |
| HMG1 Δ 04y | HMG1 (1191-1165) | HMG1 (1267-1293) | pYHMG04X | #6 | 1192˜1266 | SEQ ID NO:16 |
| HMG1 Δ 05y | HMG1 (1380-1354) | HMG1 (1573-1599) | pYHMG05X | #7 | 1381˜1572 | SEQ ID NO:17 |
| HMG1 Δ 06y | HMG1 (558-532) | HMG1 (1267-1293) | pYHMG06X | #2-#6 | 559˜1266 | SEQ ID NO:18 |
| HMG1 Δ 07y | HMG1 (558-532) | HMGl (1573-1599) | pYHMG07X | #2-#7 | 559˜1572 | SEQ ID NO:19 |
| HMG1 Δ 08y | HMG1 (27-1) | HMG1 (1573-1599) | pYHMG08X | #1-#7 | 27˜1572 | SEQ ID NO:20 |
| HMG1 Δ 10y | HMG1 (27-1) | HMG1 (1816-1842) | pYHMG10X | #1-#7 (−605 aa) | 27˜1815 | SEQ ID NO:21 |
| HMG1 Δ 11y | HMG1 (27-1) | HMG1 (1891-1917) | pYHMG11X | #1-#7 (−631 aa) | 27˜1890 | SEQ ID NO:22 |
| HMG1 Δ 12y | HMGl (27-l) | HMG1 (1990-2016) | pYHMG12X | #1-#7 (−663 aa) | 27˜1989 | SEQ ID NO:23 |
| HMG1 Δ 13y | HMG1 (27-1) | HMG1 (2218-2244) | pYHMG13X | #1-#7 (−739 aa) | 27˜2217 | SEQ ID NO:24 |

| | Primer Sequence | |
|---|---|---|
| HMG1 (27-1) | 5′ TTT CAG TCC CTT GAA TAG CGG CGG CAT | SEQ ID NO:61 |
| HMGl (558-532) | 5′ GTC TGC TTG GGT TAC ATT TTC TGA AAA | SEQ ID NO:56 |
| HMG1 (799-825) | 5′ CAC AAA ATC AAG ATT GCC CAG TAT GCC | SEQ ID NO:62 |
| HMG1 (1191-1165) | 5′ AGA AGA TAC GGA TTT CTT TTC TGC TTT | SEQ ID NO:63 |
| HMG1 (1267-1293) | 5′ AAC TTT GGT GCA AAT TGG GTC AAT GAT | SEQ ID NO:64 |
| HMG1 (1380-1354) | 5′ TTG CTC TTT AAA GTT TTC AGA GGC ATT | SEQ ID NO:65 |
| HMG1 (1573-1599) | 5′ CAT ACC AGT TAT ACT GCA GAC CAA TTG | SEQ ID NO:57 |
| HMG1 (1816-1842) | 5′ GCA TTA TTA AGT AGT GGA AAT ACA AAA | SEQ ID NO:66 |
| HMG1 (1891-1917) | 5′ CCT TTG TAC GCT TTG GAG AAA AAA TTA | SEQ ID NO:67 |
| HMG1 (1990-2016) | 5′ TCT GAT CGT TTA CCA TAT AAA AAT TAT | SEQ ID NO:68 |
| HMG1 (2218-2244) | 5′ AAG GAT GGT ATG ACA AGA GGC CCA GT/ | SEQ ID NO:69 |

In the present invention, an IPP biosynthetic pathway-related gene or a mutant thereof may be ligated to other gene or a mutant thereof so that a fusion protein is produced. In the present invention, such a gene constructed from two or more genes so that a fusion protein is produced as an expression product is called a “fusion gene”. A fusion gene may be regarded as a mutant gene in which about 1000 nucleotides are added to the nucleotide sequence of one of the genes composing the fusion gene. Accordingly, the polypeptide encoded by the fusion gene may also be regarded as a mutant polypeptide in which more than 300 amino acids are added. In order to create a fusion gene, a method may be employed in which one DNA is digested with an appropriate restriction enzyme, followed by ligation to the other DNA predigested with the same restriction enzyme in such a manner that no shift occurs in the reading frames of the amino acid sequences encoded by the DNAs. An artificial nucleotide sequence may be inserted freely into the junction site between the genes composing a fusion gene so that individual translation products of the genes can take conformations that allow them to function appropriately. For example, 5′ GGGTCC 3′ encoding Gly Ser (“GS” in FIG. 29) may be used for this purpose.

Further, in the present invention, a gene may be prepared and used that encodes a polypeptide modified so that the protein produced by the expression of an IPP biosynthetic pathway-related enzyme gene, a mutant thereof or the above-described fusion gene is directed to the endoplasmic reticulum. As endoplasmic reticulum transition polypeptides, His Asp Glu Leu (SEQ ID NO: 30) or Asp Asp Glu Leu (SEQ ID NO: 31) is known in Saccharomyces cerevisiae as a C-terminal ER retention signal; and Lys Asp Glu Leu (SEQ ID NO: 32) is known as a C-terminal signal in eukaryotes in general; it is known that they work as an ER retention signal completely equivalent to them in function (B. Lewin, Genes V, (1994), Oxford University Press, New York, U.S.A., pp. 279-318; B. Alberts et al., Molecular Biology of The Cell, third edition (1994), Garland Publishing Inc., New York, U.S.A., §12-§13). In addition to these ER retention signals consisting of the 4 amino acid residues at the C-terminal, other signal peptides from a part of the domains of ER transition proteins, e.g., the signal peptide ⁺H₃N Met Met Ser Phe Val Ser Leu Leu Leu Val Gly Ile Leu Phe Trp Ala Thr Glu Ala Glu Gln Leu Thr Lys Cys Glu Val Phe Gln (SEQ ID NO: 33) located at the N-terminal that functions as a transport signal to ER, may also be used (B. Lewin, Genes V (1994), Oxford University Press, New York, U.S.A., pp. 279-318; B. Alberts et al., Molecular Biology of The Cell, third edition (1994), Garland Publishing Inc., New York, U.S.A., §12-§13). A polypeptide in which an ER signal has been added, substituted or inserted may also be regarded as a mutant polypeptide in which 4 amino acid residues are added, substituted or inserted in the corresponding wild type polypeptide. A gene encoding the mutant polypeptide may also be regarded as a mutant gene in which a nucleotide sequence of about 12 nucleotides is added, substituted or inserted in the corresponding wild type gene.

The DNA used for gene recombination is not particularly limited as long as it may be retained hereditarily in host cells. For example, plasmid DNA, bacteriophage DNA, retrotransposon DNA and artificial chromosomal DNA (YAC: yeast artificial chromosome) may be used for this purpose. With respect to recombinant DNA fragments for genomic integration, replication ability is not necessary; DNA fragments prepared by PCR or chemical synthesis may also be used for this purpose.

Specific examples of useful plasmid DNA include YCp-type E. coli-yeast shuttle vectors such as pRS413, pRS414, pRS415, pRS416, YCp50, pAUR112 or pAUR123; YEp-type E. coli-yeast shuttle vectors such as pYES2 or YEpl3; Yip-type E. coli-yeast shuttle vectors such as pRS403, pRS404, pRS405, pRS406, pAUR101 or pAUR135; E. coli-derived plasmids such as ColE plasmids (e.g., pBR322, pBR325, pUC18, pUC19, pUC118, pUC119, pTV118N, pTV119N, pBluescript, pHSG298, pHSG396 or pTrc99A), p15A plasmids (e.g., pACYC177 or pACYC184) and pSC101 plasmids (e.g., pMW118, pMW119, pMW218 or pMW219); and Bacillus subtilis-derived plasmids (e.g., pUB110, pTP5). Specific examples of useful phage DNA include λ phage (Charon4A, Charon21A, EMBL3, EMBL4, λgt10, λgt11, λZAP), φX174, M13 mp18 and M13 mp19. Specific examples of useful retrotransposon DNA include Ty factor. Specific examples of useful YAC vectors include pYACC2.

When recombinant DNAs are introduced into hosts, selection marker genes are used in many cases. However, marker genes are not necessary if there is an appropriate assay to select recombinants without using such genes.

As the transcription promoter to direct the transcription and expression of an IPP biosynthetic pathway-related enzyme gene, a mutant thereof, a fusion gene thereof, or a gene encoding a polypeptide in which an endoplasmic reticultun signal is added, substituted or inserted in the polypeptide encoded by any one of these genes, a constitutive or inducible promoter may be used. The “constitutive promoter” means a transcription promoter of a gene involved in a major metabolic pathway. Such a promoter has transcriptional activity under any growth conditions. The “inducible promoter” means a promoter whose transcriptional activity is induced only under specific growth conditions.

Any transcription promoter may be used as long as it has activity in hosts such as yeast. For example, GAL1 promoter, GAL10 promoter, TDH3 (GAP) promoter, ADH1 promoter or TEF2 promoter may be used for expression in yeast. For expression in E. coli, trp promoter, lac promoter, trc promoter or tac promoter may be used, for example.

Further, cis-elements such as an enhancer, a splicing signal, a poly A addition signal, selectable markers, etc. may be ligated to the recombinant DNA fragment, if desired. Specific examples of useful selectable markers include marker genes such as URA3, LEU2, TRP1, HIS3, ADE2 and LYS2 with non-auxotrophic phenotypes as indicators, and drug resistance genes such as Amp^{r}, Tet^{r}, Cm^{r}, Km^{r}, A UR1-C and can1.

A transcription terminator derived from any gene may be used as long as it has activity in hosts such as yeast. For expression in yeast, ADH1 terminator or CYC1 terminator may be used, for example.

Expression vectors prepared in the present invention as recombinant DNAs for gene transfer may be designated and identified by indicating the name of the relevant gene after the name of the plasmid used. For example, the relation between the designations of expression vectors and their constitution when plasmid pRS434GAP or pRS434GAP is used is shown in Table 2 below. When HMG1 gene is ligated to plasmid pRS434GAP, for example, the resultant expression vector is expressed as “pRS434GAP-HMG1”. This method of expression also applies to cases where plasmid pRS434, pRS444, pRS435GAP or pRS445 is used in combination with the above-mentioned promoters.

TABLE 2

| Designation of | |
|---|---|
| Expression Vector | Constitution |
| pRS435GG or | Plasmid pRS435GAP to which GGPP synthase gene BTS1 is ligated |
| pRS435GAP-BTS1 | |
| pRS435F or | Plasmid pRS435GAP to which FPP synthase gene ERG20 is ligated |
| pRS435GAP-ERG20 | |
| pRS435GAP-ERG19 | Plasmid pRS435GAP to which diphosphomevalonate decarboxylase |
| | gene ERG19 is ligated |
| pRS434GAP-HMG1 | Plasmid pRS434GAP to which HMG-CoA reductase gene HMG1 is |
| | ligated |
| pRS434GAP-HMG1Δ | Plasmid pRS434GAP to which deletion mutant gene HMG1Δ of |
| | HMG-CoA reductase gene HMG1 is ligated |
| pRS435GGF | Plasmid pRS435GAP to which a fusion gene where GGPP synthase |
| | gene BTS1 and FPP synthase gene ERG20 are ligated in this order |
| | is ligated |
| pRS435FGG | Plasmid pRS435GAP to which a fusion gene where FPP synthase |
| | gene ERG20 and GGPP synthase gene BTS1 are ligated in this order |
| | is ligated |
| pRS435GGHDEL | Plasmid pRS435GG to which a nucleotide sequence encoding |
| | HDEL sequence is ligated |
| pRS435FHDEL | Plasmid pRS435F to which a nucleotide sequence encoding |
| | HDEL sequence is ligated |
| pRS435FGGHDEL | Plasmid pRS435FGG to which a nucleotide sequence encoding |
| | HDEL sequence is ligated |
| pRS435GGFHDEL | Plasmid pRS435GGF to which a nucleotide sequence encoding |
| | HDEL sequence is ligated |

3. Preparation of Recombinants

The recombinant of the present invention can be obtained by introducing the recombinant DNA of the present invention into the above-described mutant strain (e.g., EUG strains) in such a manner that various IPP biosynthetic pathway-related genes (including mutants, fusion genes, and genes encoding polypeptides where an ER signal is added, substituted or inserted) can be expressed.

Whether the gene of interest has been transferred into the host cell or not can be confirmed by such methods as PCR (polymerase chain reaction) or Southern blot hybridization. For example, DNA is prepared from the resultant recombinant and subjected to PCR using a pair of primers specific to the transferred DNA. Subsequently, the amplified product is subjected to agarose gel electrophoresis, polyacrylamide gel electrophoresis or capillary electrophoresis, followed by staining with ethidium bromide, SYBR Green solution or the like, or detection of DNA with a UV detector. Thus, by detecting the amplified product as a single band or peak, the transferred DNA can be confirmed. Alternatively, PCR may be performed using primers labeled with a fluorescent dye or the like to detect the amplified product.

4. Production of Prenyl Alcohols

In the present invention, a prenyl alcohol(s) can be obtained by culturing the above-described mutant strain (e.g., EUG strains) and recovering the prenyl alcohol(s) from the resultant culture. Alternatively, a prenyl alcohol(s) can be obtained by preparing a recombinant by transferring into the mutant strain (e.g., EUG strains) an IPP biosynthetic pathway-related gene (including a mutant or fusion gene thereof), culturing the resultant recombinant under transcription repression conditions and recovering the prenyl alcohol(s) from the resultant culture. The term “culture” used herein means any of the following materials: culture supernatant, cultured cells or microorganisms per se, or disrupted products from cultured cells or microorganisms. The mutant strain of the invention (e.g., EUG strains) or the recombinant thereof is cultured by conventional methods used in culturing the host. As the prenyl alcohol, FOH, NOH or GGOH may be enumerated. These prenyl alcohols are accumulated in the culture independently or as a mixture.

As a medium to culture the recombinant obtained from a microorganism as s host, either a complex natural medium or a synthetic medium may be used as long as it contains carbon sources, nitrogen sources and inorganic salts assimilable by the microorganism and is capable of effective cultivation of the recombinant. As carbon sources, sugar sources may be used, for example, and they usually include glucose. These sugar sources are used at 0.1-20% (w/v) in the total, preferably at 1-7% (w/v). The glucose content of the sugar source(s) used is 10-100% (w/w), preferably 50-100% (w/w), and may be selected appropriately depending on the culture conditions (e.g., the type of the host, the type of the recombinant vector introduced, the cultivation period, etc.). Hydrocarbons such as galactose, fructose, sucrose, raffinose, lactose, starch; organic acids such as acetic acid, propionic acid; and alcohols such as ethanol, propanol may also be used together with galactose as carbon sources. In such a case, the mutant strain or recombinant of the invention may be precultured in a galactose-containing medium prior to cultivation in a glucose-containing medium. The galactose content at the time of precultivation may vary depending of various carbon sources contained in the medium. Usually, the galactose content of the sugar source is 50-100% (w/w), preferably 100% (w/w). When EUG strains are used, cultivation in a galactose-containing and non-glucose-containing medium is a non-transcription repression condition for squalene synthase gene ERG9, and cultivation in a glucose-containing medium is a transcription repression condition for squalene synthase gene ERG9. Cultivation of EUG strains in a glucose-containing medium causes a reduction in the amount of squalene synthase gene transcript having translational activity.

As a nitrogen source, ammonia; ammonium salts of inorganic or organic acids such as ammonium chloride, ammonium sulfate, ammonium acetate, ammonium phosphate; other nitrogen-containing compounds; peptone; meat extract; corn steep liquor, various amino acids, etc. may be used. As an inorganic substance, potassium dihydrogen phosphate, dipotassium hydrogen phosphate, magnesium phosphate, magnesium sulfate, sodium chloride, iron(II) sulfate, manganese sulfate, copper sulfate, calcium carbonate or the like may be used. Usually, the recombinant is cultured under aerobic conditions (e.g., shaking culture or aeration agitation culture) at 26-42° C., preferably at 30° C., for 2-7 days. The adjustment of pH is carried out using an inorganic or organic acid, an alkali solution or the like.

When a recombinant incorporating an expression vector comprising an inducible transcription promoter is cultured, a transcription inducer may be added to the medium if necessary. For example, when GAL1 promoter was used in the vector, galactose may be used as an inducer.

When cultured under the above-described conditions, the mutant strain or recombinant of the invention can produce prenyl alcohols at high yields. For the mass production of prenyl alcohols, ajar fermenter cultivation apparatus or the like may be used.

The prenyl alcohol-producing mutant cells created in the present invention are capable of growing and producing prenyl alcohols at high yields by adjusting the amount of their squalene synthase gene transcript having translational activity without addition of special, essential medium components such as ergosterol required for a squalene synthase gene-deficient strain (erg9 strain; ATCC64031), . The production efficiency of prenyl alcohols can be increased further by adding to a conventional medium such substance as terpenoids, oils or surfactants, or by increasing the concentrations of nitrogen sources or carbon sources. Specific examples of these additives include the following substances.

Terpenoids: squalene, tocopherol, IPP, DMAPP

Oils: soybean oil, fish oil, almond oil, olive oil

Surfactants: Tergitol, Triton X-305, Span 85, Adekanol LG109 (Asahi Denka), Adekanol LG294 (Asahi Denka), Adekanol LG295S (Asahi Denka), Adekanol LG297 (Asahi Denka), Adekanol B-3009A (Asahi Denka), Adekapronic L-61 (Asahi Denka)

The concentrations of oils are 0.01% (w/v) or more, preferably 1-3% (w/v). The concentrations of surfactants are 0.005-1% (w/v), preferably 0.05-0.5% (w/v). The concentrations of terpenoids are 0.01% (w/v) or more, preferably 1-3% (w/v).

After the cultivation, the prenyl alcohol of interest is recovered by disrupting microorganisms or cells by, e.g., homogenizer treatment, if the alcohol is produced within the microorganisms or cells. Alternatively, the alcohol may be extracted directly using organic solvents without disrupting the cells. If the prenyl alcohol of the invention is produced outside the microorganisms or cells, the culture broth is used as it is or subjected to centrifugation or the like to remove the microorganisms or cells. Thereafter, the prenyl alcohol of interest is extracted from the culture by, e.g., extraction with an organic solvent. If necessary, the alcohol may be further isolated and purified by various types of chromatography, etc.

In the present invention, yields of prenyl alcohols of individual EUG strains and preferable combinations of EUG strains and vectors with respect to prenyl alcohol production are as illustrated in Table 3 below.

TABLE 3

| | Mevalonate | | | | | | | | | | |
|---|---|---|---|---|---|---|---|---|---|---|---|
| | pathway-related | | | | | | | | | | |
| | enzyme gene | | | | | | | | | | |
| | transferred | | | | | | | | | | |
| | (incl. mutant | | FOH | FOH | FOH | NOH | NOH | NOH | GGOH | GGOH | GGOH |
| | gene and | | Yield | Yield | Yield | Yield | Yield | Yield | Yield | Yield | Yield |
| | fusion gene) | Host | (mg/l) 1 | (mg/l) 2 | (mg/l) 3 | (mg/l) 1 | (mg/l) 2 | (mg/l) 3 | (mg/l) 1 | (mg/l) 2 | (mg/l) 3 |
| hosts | | A451 | (0.00) | | | (0.00) | | | (0.00- | | |
| | | | | | | | | | 0.02) | | |
| | | YPH499 | (0.00) | | | (0.00) | | | (0.00) | | |
| | | YPH500 | (0.00) | | | (0.00) | | | (0.00) | | |
| | | W303-1A | (0.00) | | | (0.00) | | | (0.00) | | |
| | | W303-1B | (0.00) | | | (0.00) | | | (0.00) | | |
| EUGs | | EUG5 | 0.05 | 0.05-8.5 | 8.5-57.5 | 0.05 | 0.05-0.43 | | 0.05 | 0.05-1.65 | |
| | | EUG8 | 0.05 | 0.05-9.1 | | 0.05 | 0.05-0.06 | | 0.05 | 0.05-0.17 | |
| | | EUG12 | 0.05 | 0.05-40.6 | | 0.05 | 0.05-0.36 | | 0.05 | 0.05-0.10 | |
| | | EUG24 | 0.05 | 0.05-17.9 | 17.9-42.2 | 0.05 | 0.05-1.75 | | 0.05 | 0.05-1.26 | |
| | | EUG27 | 0.05 | 0.05-18.0 | | 0.05 | | | 0.05 | | |
| | | EUG36 | 0.05 | 0.05-43 | 43 | 0.05 | 0.05-0.31 | | 0.05 | 0.05-0.98 | |
| | | EUG64 | 0.05 | 0.05-38 | 38-101.7 | 0.05 | 0.05-0.66 | | 0.05 | 0.05-2.92 | |
| HMG1 | HMG1 | EUG5 | 0.05 | 0.05-14.4 | 14.4-67.9 | 0.05 | 0.05-0.48 | 0.48-0.88 | 0.05 | 0.05-2.68 | |
| | HMG1 | EUG8 | 0.05 | 0.05-8.1 | | 0.05 | 0.05-0.07 | | 0.05 | 0.05-0.16 | |
| | HMG1 | EUG12 | 0.05 | 0.05-18.3 | 18.3-21.9 | 0.05 | 0.05-0.62 | 0.62-1.67 | 0.05 | 0.05-2.05 | |
| | HMG1 | EUG24 | 0.05 | 0.05-8.1 | 8.1-23.7 | 0.05 | 0.05-0.46 | | 0.05 | 0.05-0.94 | |
| | HMG1 | EUG27 | 0.05 | 0.05-13.6 | | 0.05 | 0.05-0.42 | | 0.05 | 0.05-2.05 | |
| | HMG1 | EUG36 | 0.05 | 0.05-2.9 | 2.9-17.8 | 0.05 | 0.05-0.31 | | 0.05 | 0.05-0.97 | |
| | HMG1 | EUG64 | 0.05 | 0.05-12.3 | 12.3-42.8 | 0.05 | 0.05-0.38 | | 0.05 | 0.05-1.58 | |
| HMG1Δ | HMG Δ026 | EUG5 | 0.05 | 0.05-3.2 | | 0.05 | 0.05-0.07 | | 0.05 | 0.05-0.09 | |
| | HMG Δ044 | EUG5 | 0.05 | 0.05-8.7 | | 0.05 | 0.05-0.07 | | 0.05 | 0.05-0.09 | |
| | HMG Δ056 | EUG5 | 0.05 | 0.05-8.7 | | 0.05 | 0.05-0.07 | | 0.05 | 0.05-0.11 | |
| | HMG Δ062 | EUG5 | 0.05 | 0.05-5.6 | | 0.05 | 0.05-0.06 | | 0.05 | 0.05-0.13 | |
| | HMG Δ076 | EUG5 | 0.05 | 0.05-8.1 | | 0.05 | 0.05-0.07 | | 0.05 | 0.05-0.15 | |
| | HMG Δ081 | EUG5 | 0.05 | 0.05-9.8 | | 0.05 | 0.05-0.10 | | 0.05 | 0.05-0.14 | |
| | HMG Δ100 | EUG5 | 0.05 | 0.05-6.6 | | 0.05 | 0.05-0.11 | | 0.05 | 0.05-0.18 | |
| | HMG Δ112 | EUG5 | 0.05 | 0.05-12.1 | | 0.05 | 0.05-0.10 | | 0.05 | 0.05-0.34 | |
| | HMG Δ122 | EUG5 | 0.05 | 0.05-8.2 | | 0.05 | 0.05-0.08 | | 0.05 | 0.05-0.13 | |
| | HMG Δ133 | EUG5 | 0.05 | 0.05-6.3 | | 0.05 | 0.05-0.08 | | 0.05 | 0.05-0.71 | |
| | HMG Δ026 | EUG12 | 0.05 | 0.05-13.8 | | 0.05 | 0.05-0.38 | | 0.05 | 0.05-0.63 | |
| | HMG Δ044 | EUG12 | 0.05 | 0.05-10.7 | | 0.05 | 0.05-0.28 | | 0.05 | 0.05-0.44 | |
| | HMG Δ056 | EUG12 | 0.05 | 0.05-9.7 | | 0.05 | 0.05-0.22 | | 0.05 | 0.05-0.40 | |
| | HMG Δ062 | EUG12 | 0.05 | 0.05-10.7 | | 0.05 | 0.05-0.25 | | 0.05 | 0.05-0.45 | |
| | HMG Δ076 | EUG12 | 0.05 | 0.05-14.4 | | 0.05 | 0.05-0.34 | | 0.05 | 0.05-0.55 | |
| | HMG Δ081 | EUG12 | 0.05 | 0.05-13.5 | | 0.05 | 0.05-0.33 | | 0.05 | 0.05-0.49 | |
| | HMG Δ100 | EUG12 | 0.05 | 0.05-12.4 | | 0.05 | 0.05-0.33 | | 0.05 | 0.05-0.44 | |
| | HMG Δ112 | EUG12 | 0.05 | 0.05-13.1 | | 0.05 | 0.05-0.33 | | 0.05 | 0.05-0.53 | |
| | HMG Δ122 | EUG12 | 0.05 | 0.05-13.6 | | 0.05 | 0.05-0.37 | | 0.05 | 0.05-0.50 | |
| | HMG Δ133 | EUG12 | 0.05 | 0.05-12.0 | | 0.05 | 0.05-0.27 | | 0.05 | 0.05-0.44 | |
| BTS1 | BTS1 | EUG8 | 0.05 | 0.05-3.9 | | | | | 0.05 | 0.05-1.42 | |
| | BTS1 | EUG12 | 0.05 | 0.05-10.6 | | 0.05 | 0.05-0.65 | | 0.05 | 0.05-6.4 | |
| | BTS1 | EUG27 | 0.05 | 0.05-7.0 | | 0.05 | 0.05-0.67 | | 0.05 | 0.05-4.4 | |
| | BTS1-HDEL | EUG5 | 0.05 | 0.05-5.2 | | 0.05 | 0.05-0.10 | | 0.05 | 0.05-0.40 | |
| | BTS1-HDEL | EUG12 | 0.05 | 0.05-9.7 | | 0.05 | 0.05-0.44 | | 0.05 | 0.05-0.73 | |
| ERG20 | ERG20 | EUG8 | 0.05 | 0.05-1.5 | | — | | | — | | |
| | ERG20 | EUG12 | 0.05 | 0.05-10.6 | | 0.05 | 0.05-0.90 | | 0.05 | 0.05-6.6 | |
| | ERG20 | EUG27 | 0.05 | 0.05-3.47 | | 0.05 | 0.05-0.38 | | 0.05 | 0.05-1.37 | |
| | ERG20-HDEL | EUG5 | 0.05 | 0.05-6.8 | | 0.05 | 0.05-0.10 | | 0.05 | 0.05-0.17 | |
| | ERG20-HDEL | EUG12 | 0.05 | 0.05-11.0 | | 0.05 | 0.05-0.62 | | 0.05 | 0.05-0.09 | |
| GGF fusion | BTS1-ERG20 | EUG5 | 0.05 | 0.05-22.6 | 22.6-28.9 | 0.05 | 0.05-0.68 | 0.68-0.75 | 0.05 | 0.05-7.3 | 7.3-10.1 |
| | BTS1-ERG20 | EUG12 | 0.05 | 0.05-7.8 | 4.0-10.5 | 0.05 | 0.05-0.60 | 0.60-0.77 | 0.05 | 0.05-5.4 | 1.6-7.0 |
| | BTS1-ERG20- | EUG5 | 0.05 | 0.05-25.3 | 7.4-25.3 | 0.05 | 0.05-0.64 | 0.43-0.64 | 0.05 | 0.05-7.0 | 7.0-17.6 |
| | HDEL | | | | | | | | | | |
| | BTS1-ERG20- | EUG12 | 0.05 | 0.05-5.0 | 5.0-12.6 | 0.05 | 0.05-0.42 | 0.42-0.92 | 0.05 | 0.05-5.6 | 5.6-8.0 |
| | HDEL | | | | | | | | | | |
| FGG fusion | ERG20-BTS1 | EUG5 | 0.05 | 0.05-2.6 | | 0.05 | 0.05-0.09 | | 0.05 | 0.05-2.43 | |
| | ERG20-BTS1 | EUG12 | 0.05 | 0.05-8.3 | | 0.05 | 0.05-0.49 | | 0.05 | 0.05-4.8 | |
| | ERG20-BTS1- | EUG5 | 0.05 | 0.05-4.2 | | 0.05 | 0.05-0.16 | | 0.05 | 0.05-0.79 | |
| | HDEL | | | | | | | | | | |
| | ERG20-BTS1- | EUG12 | 0.05 | 0.05-8.5 | | 0.05 | 0.05-0.41 | | 0.05 | 0.05-2.4 | |
| | HDEL | | | | | | | | | | |
| HMG1&BTS1 | HMG1&BTS1 | EUG5 | 0.05 | 0.05-11.0 | 11.0-55.7 | 0.05 | 0.05-0.43 | | 0.05 | 0.05-2.8 | 2.8-19.8 |
| | HMG1&BTS1 | EUG24 | 0.05 | 0.05-2.4 | 2.4-13.1 | 0.05 | 0.05-0.19 | | 0.05 | 0.05-1.1 | 1.1-4.8 |
| | HMG1&BTS1 | EUG36 | 0.05 | 0.05-4.2 | 4.2-16.9 | 0.05 | 0.05-0.31 | | 0.05 | 0.05-2.2 | 2.2-26.9 |
| | HMG1&BTS1 | EUG64 | 0.05 | 0.05-3.5 | 3.5-17.4 | 0.05 | 0.05-0.38 | | 0.05 | 0.05-1.1 | 1.1-62.7 |
| ERG19 | ERG19 | EUG5 | 0.05 | 0.05-8.9 | | 0.05 | 0.05-0.08 | | 0.05 | 0.10 | |
| | ERG19 | EUG12 | 0.05 | 0.05-9.6 | | 0.05 | 0.05-0.29 | | 0.05 | 0.05-0.13 | |
| | ERG19 | EUG24 | 0.05 | 0.05-11.4 | | 0.05 | 0.05-0.34 | | 0.05 | 0.05-0.13 | |

In the columns of FOH, NOH and GGOH yields, columns marked with “1” show the lower limit; columns marked with “2” show preferable range, and columns marked with “3” show more preferable range.
Mark “—” means no data.

(i) A451-derived EUG5 or EUG8 produced 8.5-9.1 mg/L of FOH when cultured in YM7 medium (a glucose-containing medium), and produced 57.5 mg/L of FOH, 0.43 mg/L of NOH and 1.65 mg/L of GGOH when cultured in YPD07rich medium containing sugar sources and nitrogen sources at high concentrations and oils added thereto. YPH499-derived EUG12 produced 7.44-0.6 mg/L of FOH when cultured in YM7 medium. YPH500-derived EUG24 or EUG27 produced 4.9-18.0 mg/L of FOH when cultured in YM7 medium, and produced 42.2 mg/L of FOH, 1.75 mg/L of NOH and 1.26 mg/L of GGOH when cultured in YPD07rich medium. When W303-1A-derived EUG36 and W303-1B-derived EUG64 were cultured in the same manner in YPD07rich medium, they produced 43 mg/L and 101.7 mg/L of FOH, respectively. Each of EUG strains was precultured in a galactose-containing medium, and then cultured in a glucose-containing medium, i.e., under transcription repression conditions.

(ii) When a plasmid comprising HMG-CoA reductase gene HMG1downstream of TDH3 (GAP) promoter was prepared and introduced into EUG strains, FOH accumulation increased further in the resultant recombinant strains. In some strains, GGOH accumulation was also increased. The recombinant from EUGS produced 67.9 mg/L of FOH and 2.68 mg/L of GGOH. Increase in GGOH accumulation was also observed in recombinants from EUG12 and EUG27; both recombinants produced 2.05 mg/L of GGOH at the maximum.

(iii) When HMGΔxyy genes, which are deletion mutants of HMG1′ that is a substitution mutation-type mutant of HMG1, were transferred into EUG5, the resultant recombinants cultured in YM7 medium exhibited FOH yields higher than 8.5 mg/L achieved before the HMGΔxyy transfer. The maximum yield was 12.1 mg/L of FOH when HMGΔ122 was transferred.

(iv) When a plasmid comprising GGPP synthase gene BTS1 downstream of TDH3 (GAP) promoter was prepared and introduced into EUG strains, FOH and GGOH were produced. The recombinant from EUG5 exhibited an FOH yield of 11.0 mg/L, which was higher than 8.5 mg/L achieved before the BTS1 transfer in YM7 medium. The recombinant from EUG12 produced 1.6 mg/L of GGOH in YM7 medium; and the recombinant from EUG27 produced 1.5 mg/L of GGOH in YM7 medium. When cultured in YPD07rich medium, the recombinant from EUGS produced 19.8 mg/L of GGOH; the recombinant from EUG36 produced 26.9 mg/L of GGOH; and the recombinant from EUG64 produced 62.7 mg/L of GGOH. When a mutant type BTS1 gene encoding a mutant type GGPP synthase having an ER signal HDEL at the C-terminal was transferred, FOH productivity increased from 2.6 mg/L (the yield when wild type BTS1 was transferred) to 9.7 mg/L in the recombinant from EUG12.

(v) While ERG20-transferred YPH499 strain produced little prenyl alcohol (0.05 mg/L or less), ERG20-transferred EUG12 produced 4.5 mg/L of FOH and also exhibited a good result in GGOH production (6.6 mg/L). The transfer of a mutant type ERG20 encoding a mutant polypeptide to which HDEL is added was sometimes effective in NOH production; the recombinant from EUG12 produced 0.62 mg/L of NOH.

(vi) While the transfer of BTS1-ERG20 fusion gene or this fusion gene engineered so that one of its ends encodes HDEL sequence into A451 resulted in GGOH production of only about 0.26 mg/L, the same gene-transferred EUG5 produced 6.5-6.6 mg/L of GGOH in YM7 medium.

(vii) When ERG20-BTS1 fusion gene or this fusion gene engineered so that one of its ends encodes HDEL sequence was transferred into EUG5, the resultant recombinant also produced 0.79-2.43 mg/L of GGOH as in (vi) above.

(viii) When ERG19 was transferred into EUG strains, it appeared that FOH production was not considerably improved in any of the resultant recombinants. However, when FOH yields per OD₆₀₀ value of the culture broth were compared, FOH production efficiency were improved two- to several-fold in recombinants prepared from YPH strain-derived EUG12 and EUG24.

### BRIEF DESCRIPTION OF THE DRAWINGS

FIG. 1 is a diagram showing a metabolic pathway of IPP biosynthetic pathway-related enzymes.

FIG. 2 presents a physical map around the ERG9 locus and diagrams showing the construction of DNA fragments for replacing the ERG9 promoter region.

FIG. 3 is a physical map of an expression shuttle vector pYES2.

FIG. 4A shows patterns of substitution mutations.

FIG. 4B shows the construction of deletion mutants of HMG1′.

FIG. 5 is a photograph showing the colony formation of EUG strains on agar plate medium.

FIG. 6A presents graphs showing the prenyl alcohol production of EUG8.

FIG. 6B presents graphs showing the prenyl alcohol production of EUG12.

FIG. 6C presents graphs showing the prenyl alcohol production of EUG27.

FIG. 7 presents graphs showing the prenyl alcohol production of EUG5 which is an A451-derived EUG strain.

FIG. 8 presents graphs showing the prenyl alcohol production of EUG12 which is a YPH499-derived EUG strain.

FIG. 9 presents graphs showing the prenyl alcohol production of EUG24 and EUG27 which are YPH500-derived EUG strains.

FIG. 10A presents graphs showing the prenyl alcohol production of W303-1A-derived EUG strains.

FIG. 10B presents graphs showing the prenyl alcohol production of W303-1A-derived EUG strains.

FIG. 10C presents graphs showing the prenyl alcohol production of W303-1A-derived EUG strains.

FIG. 10D presents graphs showing the prenyl alcohol production of W303-1A-derived EUG strains.

FIG. 11A presents graphs showing the prenyl alcohol production of W303-1B-derived EUG strains.

FIG. 11B presents graphs showing the prenyl alcohol production of W303-1B-derived EUG strains.

FIG. 12A presents graphs showing the prenyl alcohol production of EUG8 cultured in media with varied initial sugar concentration/composition.

FIG. 12B presents graphs showing the prenyl alcohol production of EUG8 cultured in media with varied initial sugar concentration/composition.

FIG. 12C presents graphs showing the prenyl alcohol production of EUG8 cultured in media with varied initial sugar concentration/composition.

FIG. 13A presents graphs showing the prenyl alcohol production of EUG12 cultured in media with varied initial sugar concentration/composition.

FIG. 13B presents graphs showing the prenyl alcohol production of EUG12 cultured in media with varied initial sugar concentration/composition.

FIG. 13C presents graphs showing the prenyl alcohol production of EUG12 cultured in media with varied initial sugar concentration/composition.

FIG. 14A presents graphs showing the prenyl alcohol production of EUG27 cultured in media with varied initial sugar concentration/composition.

FIG. 14B presents graphs showing the prenyl alcohol production of EUG27 cultured in media with varied initial sugar concentration/composition.

FIG. 14C presents graphs showing the prenyl alcohol production of EUG27 cultured in media with varied initial sugar concentration/composition.

FIG. 15A presents graphs showing the production of FOH by EUG8, EUG12 and EUG27 cultured in media with varied initial sugar concentration/composition.

FIG. 15B presents graphs showing the production of FOH by EUG8, EUG12 and EUG27 cultured in media with varied initial sugar concentration/composition.

FIG. 15C presents graphs showing the production of FOH by EUG8, EUG12 and EUG27 cultured in media with varied initial sugar concentration/composition.

FIG. 16A is a diagram showing plasmid pRS434GAP.

FIG. 16B is a diagram showing plasmid pRS435GAP.

FIG. 16C is a diagram showing plasmid pRS444GAP.

FIG. 16D is a diagram showing plasmid pRS445GAP.

FIG. 17 is a physical map of plasmid pALHMG106.

FIG. 18A is a graph showing the prenyl alcohol production of EUG strains into which ERG19 expression plasmid has been introduced.

FIG. 18B is a graph showing the prenyl alcohol production of EUG strains into which ERG19 expression plasinid has been introduced.

FIG. 19A is a graph showing the prenyl alcohol production of A451.

FIG. 19B is a graph showing the prenyl alcohol production of EUG8.

FIG. 20A is a graph showing the prenyl alcohol production of EUG8 when pRS434GAP-HMG1 has been introduced thereinto.

FIG. 20B is a graph showing the prenyl alcohol production of EUG8 when rpRS444GAP-HMG1 has been introduced thereinto.

FIG. 21 is a graph showing the prenyl alcohol production of EUG12 and EUG27.

FIG. 22A is a graph showing the prenyl alcohol production of EUG12 when pRS434GGAP-HM1G or pRS444GAP-HMG1 has been introduced thereinto.

FIG. 22B is a graph showing the prenyl alcohol production of EUG27 when pRS434GGAP-HMG1 or pRS444GAP-HMG1 has been introduced thereinto.

FIG. 23A is a graph showing prenyl alcohol yields of EUG5 when plasmids for expressing deletion mutants of HMG1 gene have been introduced thereinto.

FIG. 23B is a graph showing prenyl alcohol yields of EUG5 when plasmids for expressing deletion mutants of HMG1 gene have been introduced thereinto.

FIG. 24A is a graph showing prenyl alcohol yields of EUG12 when plasmids for expressing deletion mutants of HMG1 gene have been introduced thereinto.

FIG. 24B is a graph showing prenyl alcohol yields of EUG12 when plasmids for expressing deletion mutants of HMG1 gene have been introduced thereinto.

FIG. 25A is a graph showing prenyl alcohol yields of EUG8 when ERG20 gene expression plasmid has been introduced thereinto.

FIG. 25B is a graph showing prenyl alcohol yields of EUG8 when ERG20 gene expression plasmid has been introduced thereinto.

FIG. 26A is a graph showing prenyl alcohol yields of EUG12 when ERG20 gene expression plasmid has been introduced thereinto.

FIG. 26B is a graph showing prenyl alcohol yields of EUG27 when ERG20 gene expression plasmid has been introduced thereinto.

FIG. 27A is a graph showing prenyl alcohol yields of EUG8 when BTS1 gene expression plasmid pRS435GAP-BTS1 has been introduced thereinto.

FIG. 27B is a graph showing prenyl alcohol yields of EUG8 when BTS1 gene expression plasmid pRS445GAP-BTS1 has been introduced thereinto.

FIG. 28A is a graph showing prenyl alcohol yields of EUG12 when BTS1 gene expression plasmid has been introduced thereinto.

FIG. 28B is a graph showing prenyl alcohol yields of EUG27 when BTS1 gene expression plasmid has been introduced thereinto.

FIG. 29 shows primers used in the preparation of BTS1-ERG20 fusion genes and genes encoding polypeptide having an ER signal at the C-terminal, as well as the locations and directions of such primers.

FIG. 30 shows the results of determination of GGOH yields in YM7 medium when A451 or EUG5 was used as a host.

FIG. 31 shows the results of determination of GGOH yields in YMO7 medium when A451 or EUG5 was used as a host.

FIG. 32 shows the results of determination of GGOH yields in YM7 medium when YPH499 or EUG12 was used as a host.

FIG. 33 shows the results of determination of GGOH yields in MYO7 medium when YPH499 or EUG12 was used as a host.

FIG. 34A presents graphs showing GGOH yields of pRS435GGF- or pRS435GGFHDEL-introduced EUG5 cultured in a medium with the indicated initial sugar composition for 2 days.

FIG. 34B presents graphs showing GGOH yields of pRS435GGF- or pRS435GGFHDEL-introduced EUG5 cultured in a medium with the indicated initial sugar composition for 4 days.

FIG. 34C presents graphs showing GGOH yields of pRS435GGF- or pRS435GGFHDEL-introduced EUG5 cultured in a medium with the indicated initial sugar composition for 7 days.

FIG. 35A presents graphs showing GGOH yields of pRS435GGF- or pRS435GGFHDEL-introduced EUG12 cultured in a medium with the indicated initial sugar composition for 2 days.

FIG. 35B presents graphs showing GGOH yields of pRS435GGF- or pRS435GGFHDEL-introduced EUG12 cultured in a medium with the indicated initial sugar composition for 4 days.

FIG. 35C presents graphs showing GGOH yields of pRS435GGF- or pRS435GGFHDEL-introduced EUG12 cultured in a medium with the indicated initial sugar composition for 7 days.

FIG. 36A presents graphs showing the time course of prenyl alcohol yields and OD₆₀₀ values when EUG5 and recombinants derived therefrom were cultured in YPD)7rich medium.

FIG. 36B presents graphs showing the time course of prenyl alcohol yields and OD₆₀₀ values when EUG24 and recombinants derived therefrom were cultured in YPDO7rich medium.

FIG. 36C presents graphs showing the time course of prenyl alcohol yields and OD₆₀₀ values when EUG36 and recombinants derived therefrom were cultured in YPDO7rich medium.

FIG. 36D presents graphs showing the time course of prenyl alcohol yields and OD₆₀₀ values when EUG64 and recombinants derived therefrom were cultured in YPDO7rich medium.

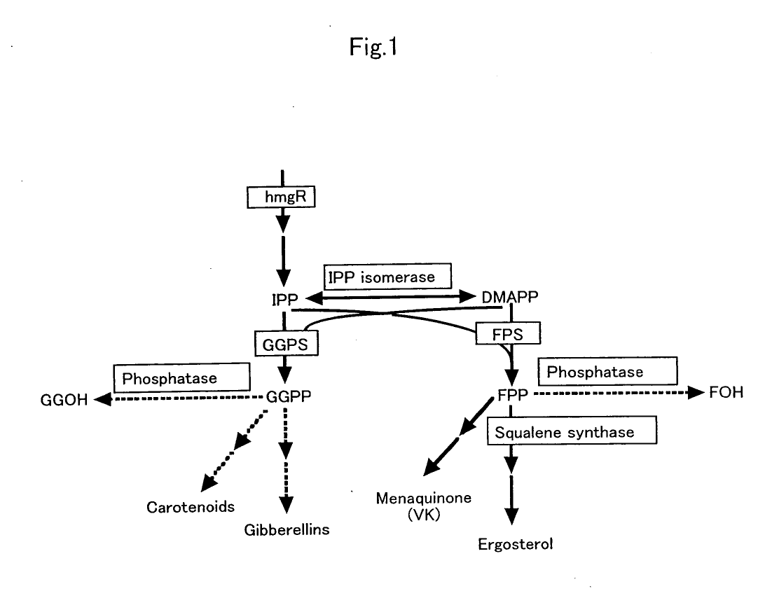

### BEST MODE FOR CARRYING OUT THE INVENTION

Hereinbelow, the present invention will be described in more detail with reference to the following Examples. However, the technical scope of the invention is not limited to these Examples.

The Examples encompass the following contents.

(1) Mutant strains (EUG strains) are prepared by replacing the transcription promoter for the squalene synthase gene in their genome with GAL1 promoter (a transcription repression type promoter), and used as prenyl alcohol-producing microorganisms. The resultant EUG strains are cultured under transcription repression conditions (i.e., in a glucose-containing medium), followed by determination of the yields of prenyl alcohols. [Examples 1-2]

(2) Expression vectors pRS435GAP and so forth are prepared based on pRS vectors (Stratagene), pYES vectors (Invitrogen) or genomic DNA from E. coli, for transferring genes into EUG strains. [Example 3]

(3) Cloning of IPP Biosynthetic Pathway-Related Genes

As genes to be transferred into the above EUG strains, HMG-CoA reductase gene, diphosphomevalonate decarboxylase gene, FPP synthase gene, GGPP synthase gene, and mutants thereof are cloned, followed by preparation of expression plasmids. [Examples 4-6 and 8]

(4) Recombinants into which the genes cloned in (3) above were transferred are cultured under transcription repression conditions (i.e., in a glucose-containing medium), followed by determination of the yields of prenyl alcohols. [Examples 7-8]

(5) EUG strains are cultured in media with varied glucose concentration or media to which sugar sources, nitrogen sources and oil additives were added, to thereby examine condisions suitable for prenyl alcohol production. [Examples 9-10]

### EXAMPLE 1

### Preparation of EUG Strains

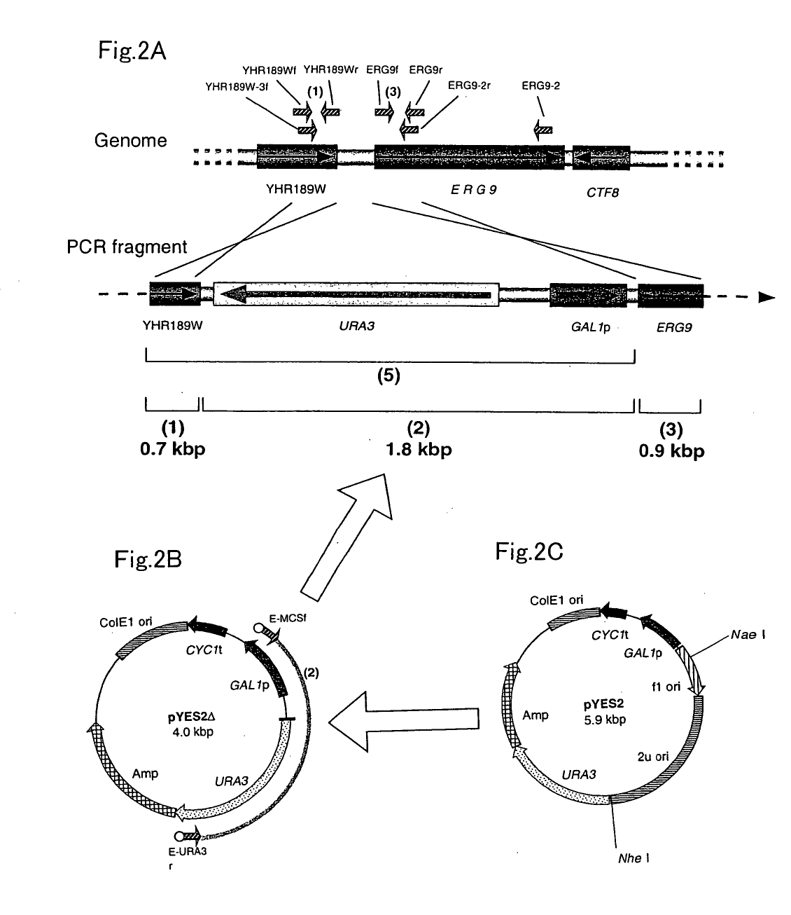

A gene map around squalene synthase gene ERG9 was obtained from SGD. Based on this map, PCR primer DNAs for amplifying DNA fragments for replacing ERG9 transcription promoter (ERG9p) were designed (FIG. 2). On the other hand, a 1.8 kbp DNA fragment (FIG. 2A, (2)) comprising a transformant selection marker gene URA3 and a transcription promoter GAL1p was prepared by PCR amplification using, as a template, pYES2Δ (FIG. 2B) obtained by digesting pYES2 (FIG. 2C) with NaeI and NheI, blunt-ending with Klenow enzyme and deleting 2μ ori by self-ligation.

The primers used in the PCR are as follows.

| | | |
|---|---|---|
| E-MCSf: | 5′-GCC GTT GAC AGA GGG TCC GAG CTC GGT ACC AAG-3′ | (SEQ ID NO:34) |
| E-URA3r: | 5′-CAT ACT GAC CCA TTG TCA ATG GGT AAT AAC TGA T-3′ | (SEQ ID NO:35) |

In each of the above primers, an Eam1105 I recognition site (the underlined portion) was added so that a 0.7 kbp DNA fragment comprising a downstream portion of the open reading frame YHR189W in the genome of S. cerevisiae and a 0.9 kbp DNA fragment comprising an upstream portion of ERG9 can be carried out T/A ligation. The YHR189W fragment (i) was prepared by PCR using the following primers YHR189Wf and YHR189Wr, and YPH499 genomic DNA as a template. The ERG9 fragment (ii) was prepared by PCR using the following primers ERG9f and ERG9r, and YPH499 genomic DNA as a template. YPH499 genomic DNA was prepared with a yeast genomic DNA preparation kit “Dr. GenTLE™” (Takara).

| | |
|---|---|
| YHR189Wf: | |
| 5′-TGT CCG GTA AAT GGA GAC-3′ | (SEQ ID NO:36) |
| YHR189Wr: | |
| 5′-TGT TCT CGC TGC TCG TTT-3′ | (SEQ ID NO:37) |
| ERG9f: | |
| 5′-ATG GGA AAG CTA TTA CAA T-3′ | (SEQ ID NO:38) |
| ERG9r: | |
| 5′-CAA GGT TGC AAT GGC CAT-3′ | (SEQ ID NO:39) |

The 1.8 kbp DNA fragment was digested with Eam1105I and then ligated to the 0.7 kbp DNA fragment. With the resultant fragment as a template, 2nd PCR was carried out using the above-described primers YHR189Wf and E-MCSf. The amplified 2.5 kbp DNA fragment (FIG. 2A, (5)) was digested with Eam1105I and then ligated to the 0.9 kbp fragment. With the resultant fragment as a template, 3rd PCR was carried out using the following primers YHR189W-3f and ERG9-2r. As a result, a 3.4 kbp DNA fragment was amplified. This was used as a DNA fragment for transformation.

| | |
|---|---|
| YHR189W-3f: | |
| 5′-CAA TGT AGG GCT ATA TAT G-3′ | (SEQ ID NO:40) |
| ERG9-2r: | |
| 5′-AAC TTG GGG AAT GGC ACA-3′ | (SEQ ID NO:41) |

The DNA fragment for transformation was introduced into yeast strains using Frozen EZ Yeast Transformation II kit purchased from Zymo Research (Orange, Calif.). As host strains for the DNA transfer, A451, YPH499, YPH500, W303-1A and W303-1B were used.

The resultant recombinants were cultured on an agar medium called SGR-U medium that had been obtained by adding CSM-URA (purchased from BIO 101, Vista, Calif.) and adenine sulfate (final concentration 40 mg/L) to SGR medium [a variation of SD (synthetic dextrose) medium where the glucose component is replaced with galactose and raffinose] at 30° C. Colonies grown on the medium were spread on the same medium again, cultured and then subjected to single colony isolation.

The resultant recombinants were designated EUG (ERG9p::URA3-GAL1p) strains. Of these, clones derived from A451 were designated EUG1 through EUG10; clones derived from YPH499 were designated EUG11 through EUG20; clones derived from YPH500 were designated EUG21 through EUG30; clones derived from W303-1A were designated EUG31 through EUG50; and clones derived from W303-1B were designated EUG51 through EUG70.

These clones were cultured on SD medium to select those clones that exhibit a decrease in growth rate as a result of reduction in the amount of squalene synthase gene transcript having translational activity due to glucose repression. As a result, EUG8 from A451, EUG12 from YPH499 and EUG27 from YPH500 were obtained. Representative examples of states of growth on SD-U agar medium and SGR-U agar medium are shown in FIG. 5. A clone could be obtained (in FIG. 5, EUG5 among EUG1 through EUG6) that grew normally on SGR-U plate (FIG. 5, right panel) but exhibited a decrease in growth rate on SD-U plate (FIG. 5, left panel titled “SD”) as a result of reduction in the amount of squalene synthase gene transcript having translational activity caused by the repression of transcription of the squalene synthase gene under the control of GAL1 promoter due to glucose repression.

Among those recombinants that exhibited a decrease in growth rate as a result of reduction of the amount of squalene synthase gene transcript having translational activity, EUG8, EUG12 and EUG27 were selected, followed by preparation of genomic DNA using Dr. GenTLE™. Then, PCR was carried out using the genomic DNA as a template and primers YHR189Wf and ERG9-2 (5′-TCA CGC TCT GTG TAA AGT GTA TA-3′ (SEQ ID NO: 42)), and the size of the amplified DNA fragment was examined by agarose gel electrophoresis. The results confirmed that the 1.8 kbp PCR fragment comprising URA3 and GAL1p is integrated into the squalene synthase gene locus in the genome of each strain.

### EXAMPLE 2

### Production of Prenyl Alcohols by Individual EUG Strains

(1) Determination of Prenyl Alcohol Yields: Part I

(1-1) Cultivation

EUG8, EUG12 and EUG27 were precultured in SGR-U medium, and 0.3 ml of each preculture broth was added to 30 ml of YM7 medium, YME medium or YM7 (Gal) medium. Then, each strain was cultured under reciprocal shaking at 130 rpm in a 300 ml Erlenmeyer flask at 30° C. YM7 medium refers to YM medium whose pH is adjusted to 7 with NaOH. YME medium refers to YM7 medium to which 50 mg/L of ergosterol is added. Specifically, this medium is prepared by adding 1/1000 volume of an ergosterol stock solution in ethanol-Tergitol (50 mg/ml) to YM7 medium. YM7 (Gal) medium refers to YM7 medium where its glucose component is replaced with galactose.

A 2.5 ml sample of the culture broth was taken out on day 1, 2, 3, 4, 7, 8 and 9 of the cultivation and subjected to determination of prenyl alcohol yields. Briefly, 2.5 ml of methanol was added to each sample and mixed. About 5 ml of pentane was added to this mixture, agitated vigorously and then left stationary. The resultant pentane layer was transferred into a fresh glass tube, which was then placed in a draft. Pentane was evaporated therein to condense the solute components. Subsequently, prenyl alcohols were identified and quantitatively determined by gas chromatography/mass spectrometry (GC/MS). At that time, the degree of cell growth was also examined by diluting 50 μl of each culture broth 30-fold with water and measuring the absorbance at 600 nm.

(1-2) GC/MS Analysis

The fraction extracted with pentane was separated, identified and quantitatively determined with HP6890/5973 GC/MS system (Hewlett-Packard, Wilmington, Del.). The column used was HP-5MS (0.25 mm×30 m; film thickness 0.25 μm). Analytical conditions were as described below. The same conditions were used for all the GC/MS analyses in this specification.

Inlet temperature: 250° C.

Detector temperature: 260° C.

[MS Zone Temperatures]

MS Quad: 150° C.

MS Source: 230° C.

Mass scan range: 35-200

[Injection Parameters]

Automated injection mode

Sample volume: 2 μl

Methanol washing: 3 times; hexane washing: twice

Split ratio: 1/20

Carrier gas: helium 1.0 ml/min

Solvent retardation: 2 min

[Oven Heating Conditions]

115° C. for 90 sec

Heating up to 250° C. at 70° C./min and retaining for 2 min

Heating up to 300° C. at 70° C./min and retaining for 7 min

Internal standard: 0.01 μl of 1-undecanol in ethanol

Reliable standards: (all-E)-Nerolidol (Eisai)

(all-E)-Famesol (Sigma)

(all-E)-Geranylgeraniol (Eisai)

Squalene (Tokyo Kasei Kogyo)

(2) Determination of Prenyl Alcohol Yields: Part II

Those EUG strains that exhibited a decrease in growth rate in SD medium were cultured in 1-5 ml of YM7 medium at 30° C. under reciprocal shaking at 130 rpm. A sample (0.8-2.5 ml) of the culture broth was taken out on day 1, 2, 3, 4 and 7 of the cultivation, followed by determination of the amounts of prenyl alcohols in the same manner as described in (1) above.

(3) Changes in Prenyl Alcohol Yields Depending on Sugar Composition

The glucose (Glc) component in YM7 medium was varied as indicated in Table 4 below, and EUG strains were cultured therein for 2 days at 30° C. under reciprocal shaking at 130 rpm. Subsequently, 5% (w/v) Glc was added to the YM7 medium to prepare YM Glc medium and 5% (w/v) galactose (Gal) was added to the YM7 medium to prepare YM Gal medium, as shown in Table 5, and the strains were cultured further up to day 7. With respect to YMrich medium, the total sugar concentration in YM7 was set at 6% (w/v) from the start of the cultivation, and the sugar composition therein was as shown in Table 4. The relationships between medium composition and cultivation period may be summarized as shown in Table 5. A 2.5 ml sample of the culture broth was taken out on day 2, 4 and 7 of the cultivation, followed by determination of the prenyl alcohol yields in the same manner as described in (1) above.

TABLE 4

| | Glucose | Galactose |
|---|---|---|
| No. | (%) | (%) |
| 1 | 100 | 0 |
| 2 | 75 | 25 |
| 3 | 50 | 50 |
| 4 | 20 | 80 |
| 5 | 10 | 90 |
| 6 | 5 | 95 |
| 7 | 1 | 99 |
| 8 | 0 | 100 |

TABLE 5

| Medium | Day 0 | Day 2 | Day 4 | Day 7 |
|---|---|---|---|---|
| YM Glc | 1% | 6% | 6% | 6% |
| | | (5% Glc added) | | |
| YM Gal | 1% | 6% | 6% | 6% |
| | | (5% Gal added) | | |
| YM rich | 6% | 6% | 6% | 6% |
| No. | xxx-1 | xxx-2 | xxx-3 | xxx-4 |

(4) Results and Considerations

(4-1) Penyl Alcohol Production by EUG Strains

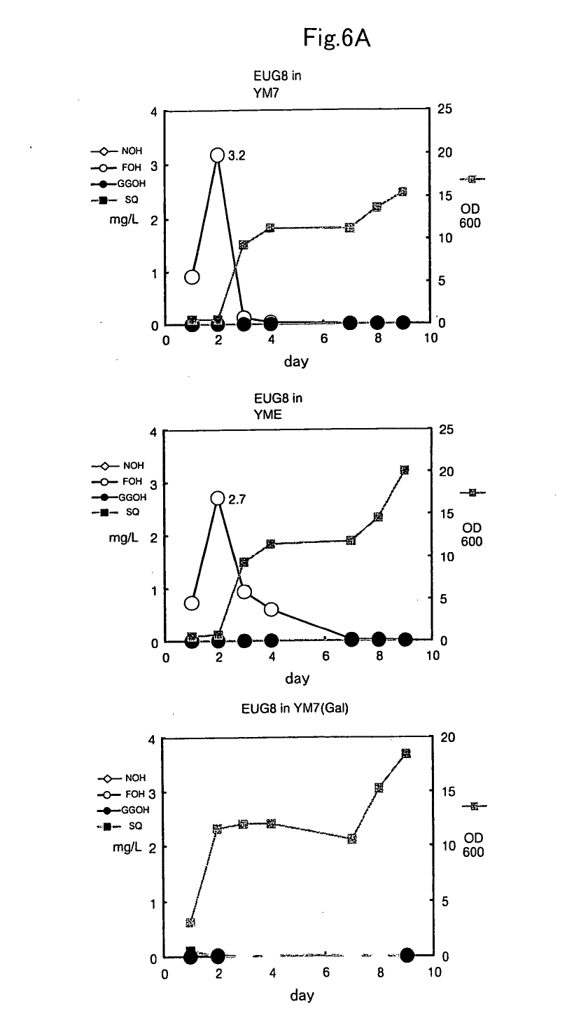

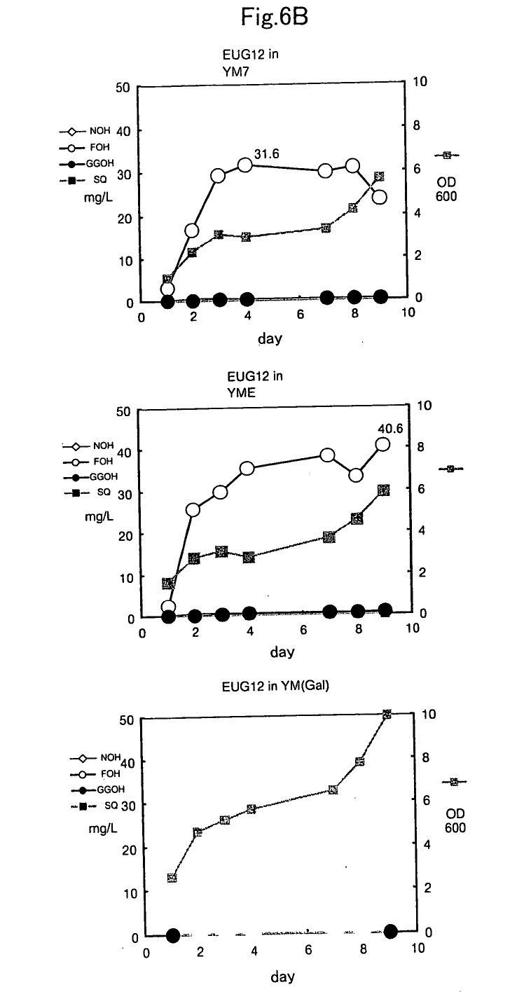

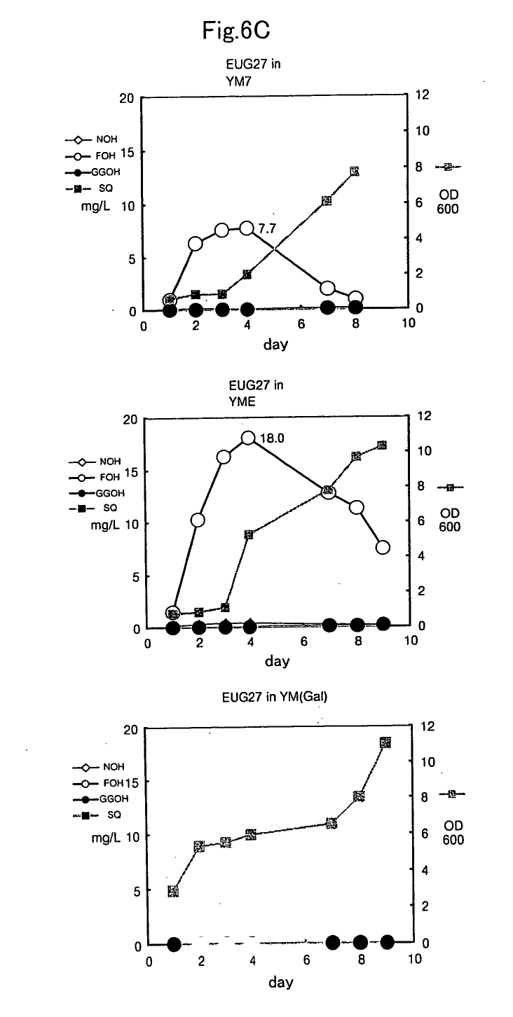

A451-, YPH499- and YPH500-derived EUG strains that exhibited a decrease in growth rate in a glucose-containing medium and had GAL1p completely integrated into the genome were selected (EUG8, EUG12 and EUG27 were selected from respective strains), and their prenyl alcohol yields were determined. The results are shown in FIGS. 6A-C. When they were cultured in YM7 or YME medium both containing Glc as a carbon source, FOH-producing systems were obtained. When cultured in YME medium containing ergosterol, EUG12 and EUG27 exhibited a tendency of increase in FOH concentration. They accumulated 40.6 mg/L and 18.0 mg/L of FOH at the maximum, respectively. A451-derived EUG8, YPH499-derived EUG12 and YPH500-derived EUG27 are different in production profile, and it is believed that YPH-derived strains are more suitable for prenyl alcohol production. Thus, a yeast clone was developed that can produce about 30 mg/L of FOH when cultured for only 4 days in the common medium YM7 (see EUG12 in FIG. 6) without addition of ergosterol (Erg) essential for the growth of conventional ERG9-defficient strains (erg9 strain: ATCC64031).

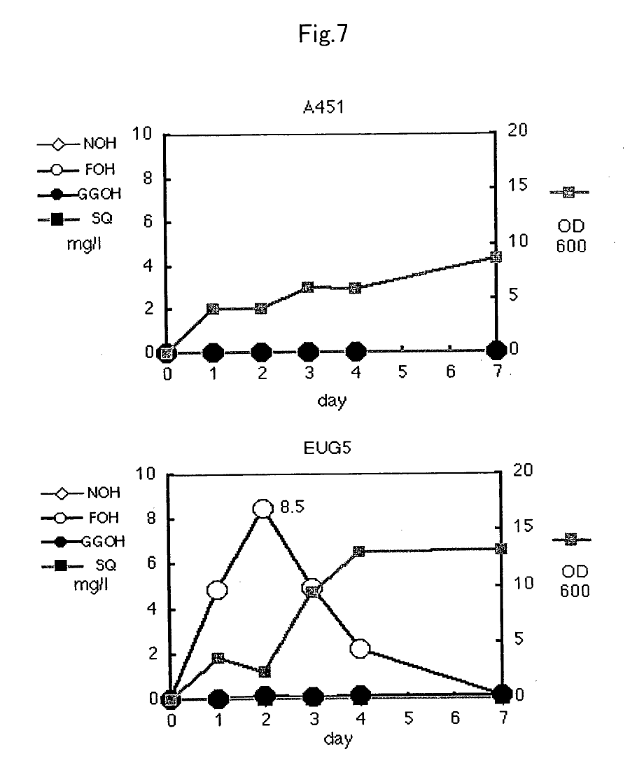

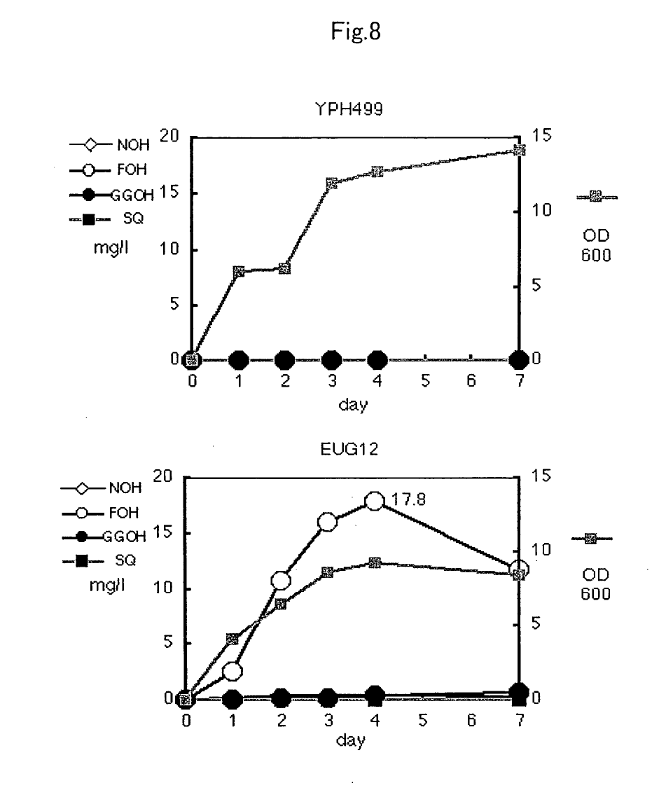

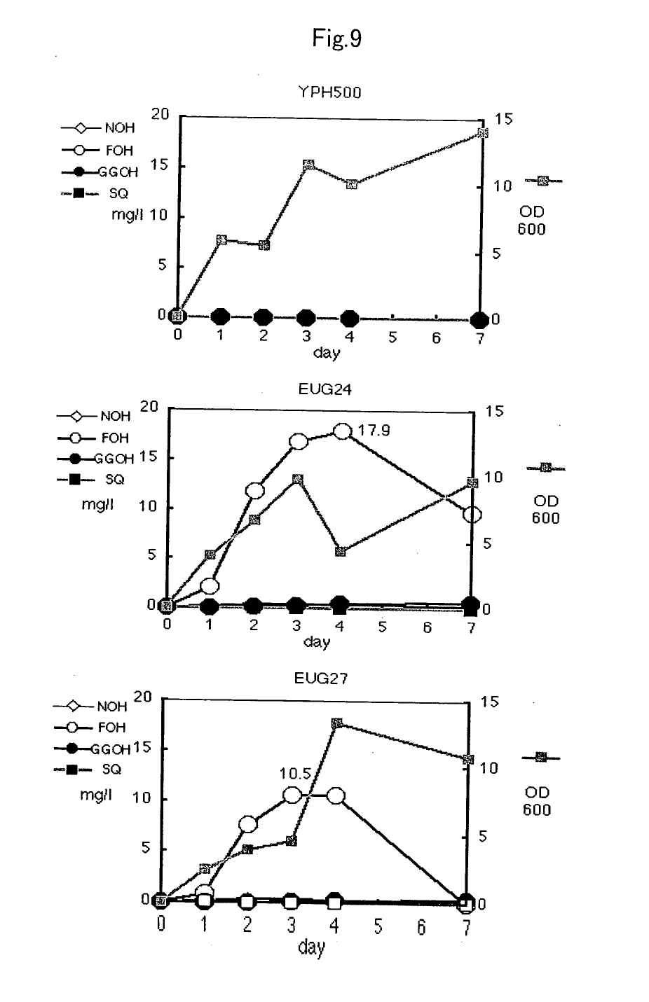

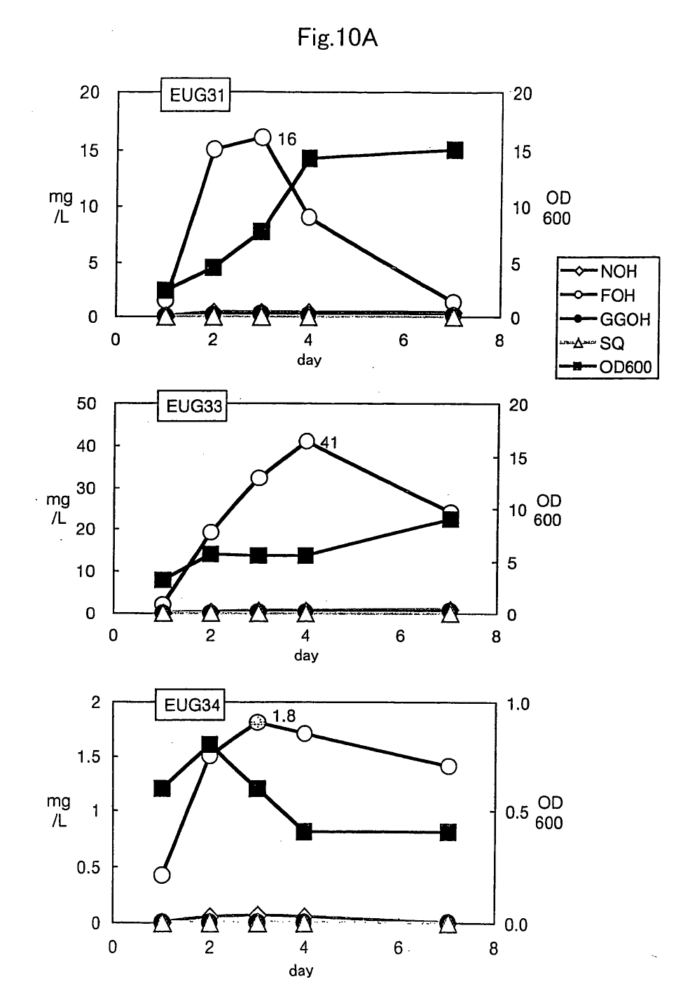

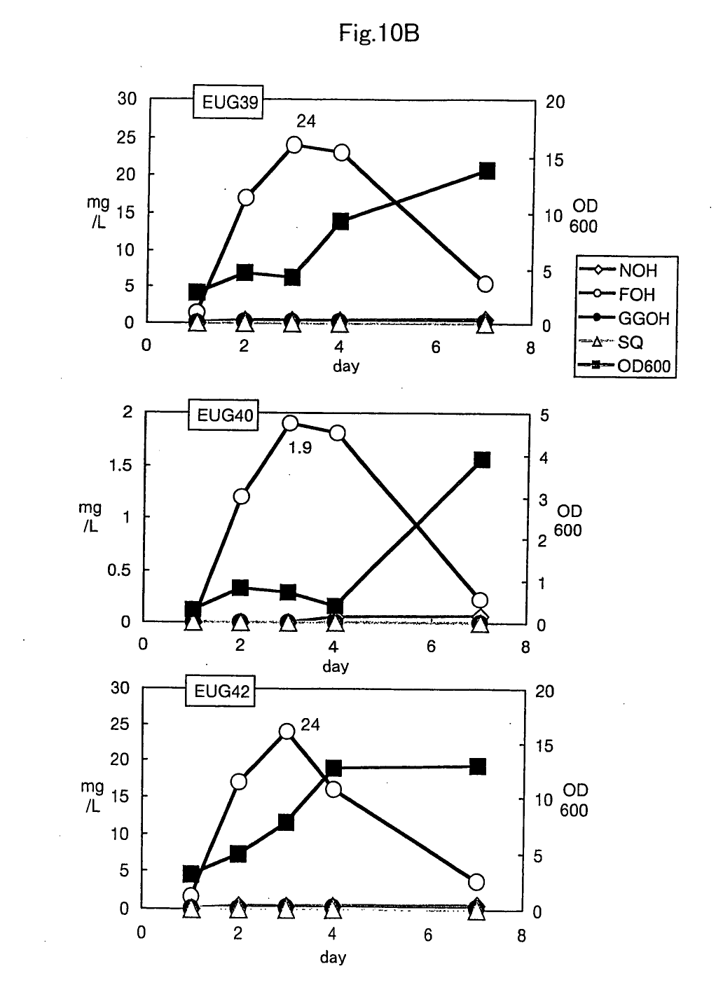

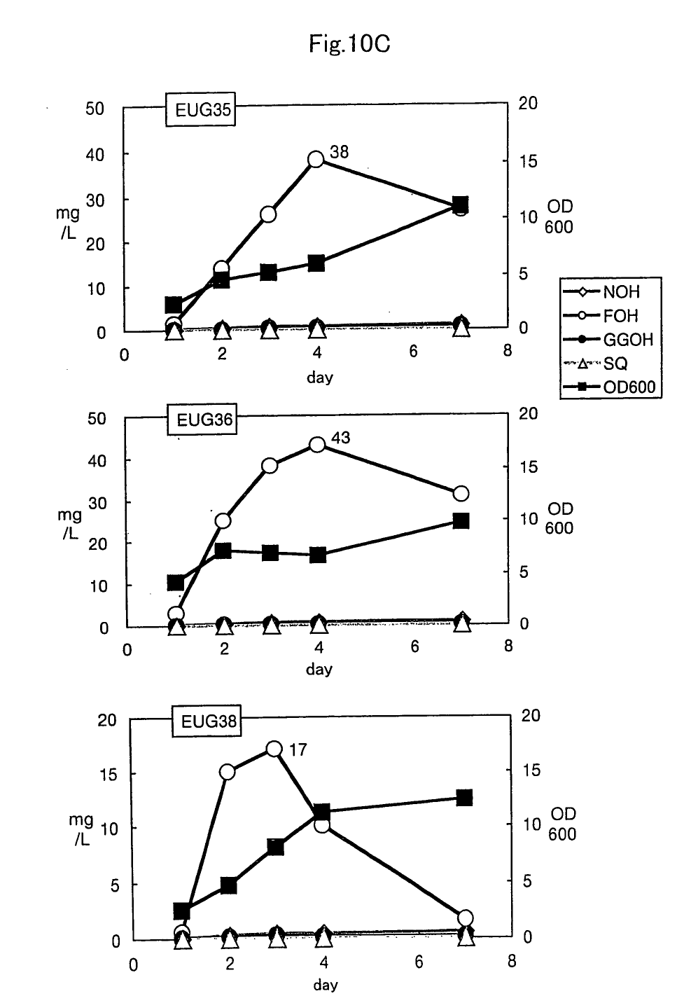

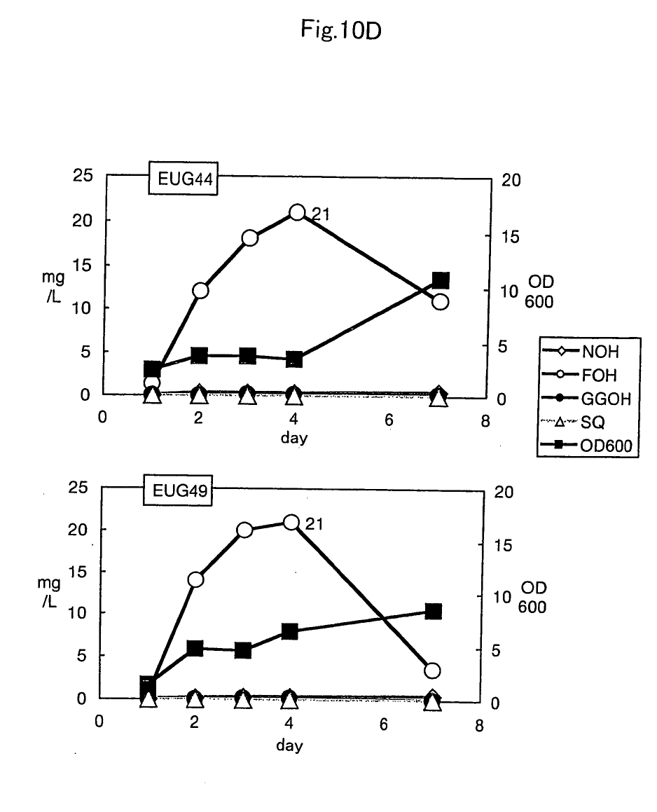

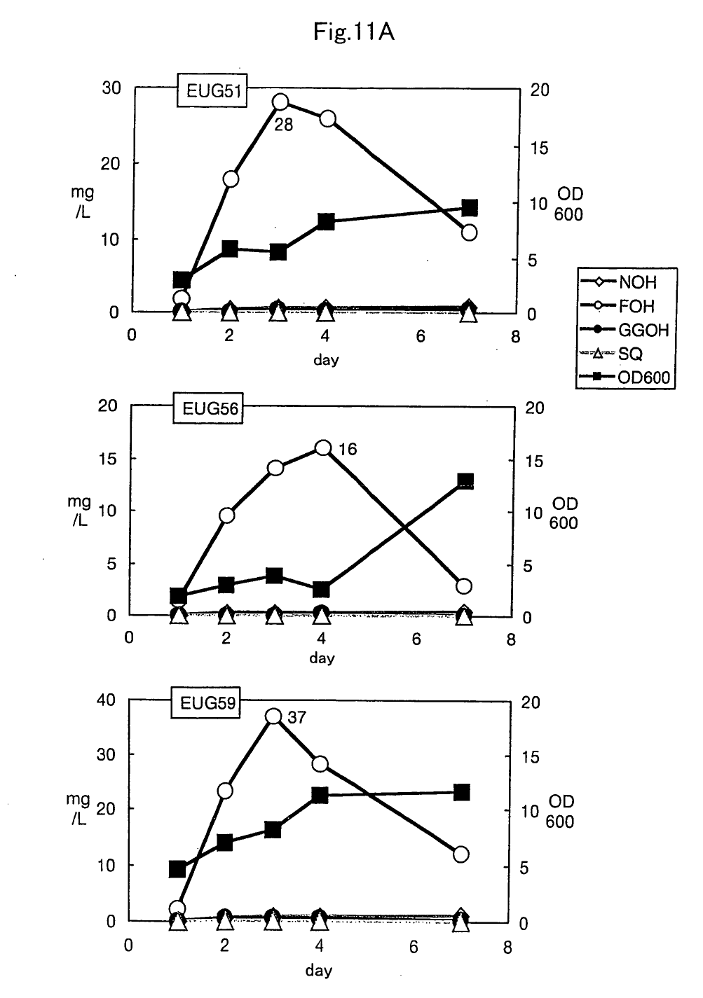

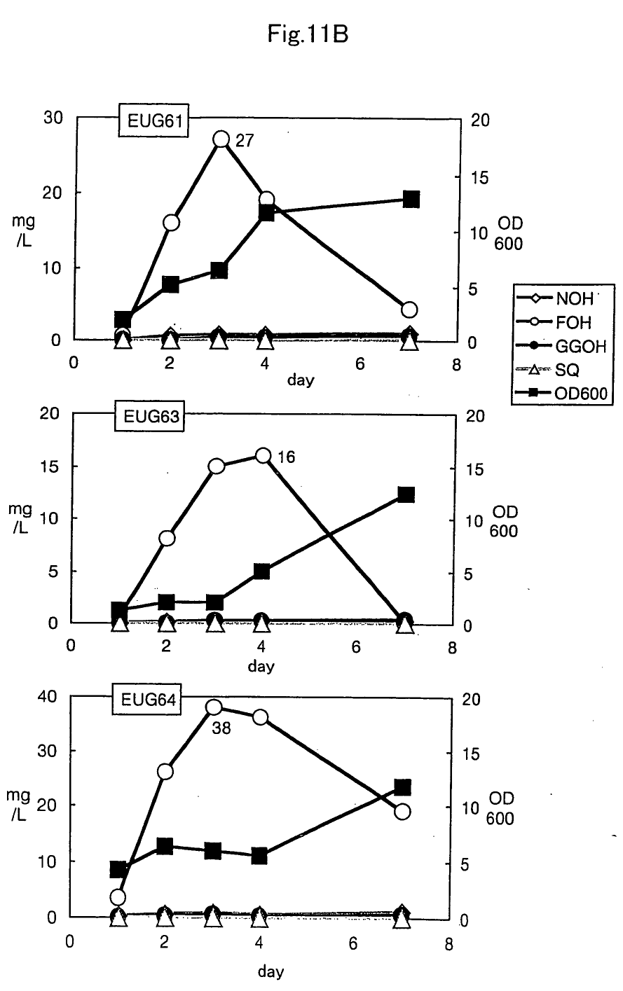

Subsequently, out of EUG1 through EUG70, those clones that exhibited a decrease in growth rate in SD medium were cultured and their prenyl alcohol yields were compared (FIGS. 7-11). With respect to the production of FOH, EUG5 exhibited a higher yield (8.5 mg/L) among A451-derived strains (FIG. 7); EUG12 exhibited a higher yield (17.8 mg/L) among YPH499-derived strains (FIG. 8); and EUG24 and EUG27 exhibited higher yields (17.9 mg/L and 10.5 mg/L, respectively) among other strains (FIG. 9). Among W303-1A-derived clones (FIGS. 10A-D) and W303-1B-derived clones (FIGS. 11A-B), the strains indicated in the respective Figures exhibited higher yields than other strains.

W303-lA-derived clones:

EUG31 (16 mg/L), EUG33 (41 mg/L), EUG35 (38 mg/L), EUG36 (43 mg/L), EUG38 (17 mg/L), EUG39 (24 mg/L), EUG42 (24 mg/L), EUG44 (21 mg/L), EUG49 (21 mg/L)

W303-1B-derived clones:

EUG51 (28 mg/L), EUG56 (16 mg/L), EUG59 (37 mg/L), EUG61 (27 mg/L), EUG63 (16 mg/L), EUG64 (38 mg/L)

(4-2) Difference in Productivity Depending on Changes in Glucose Concentration

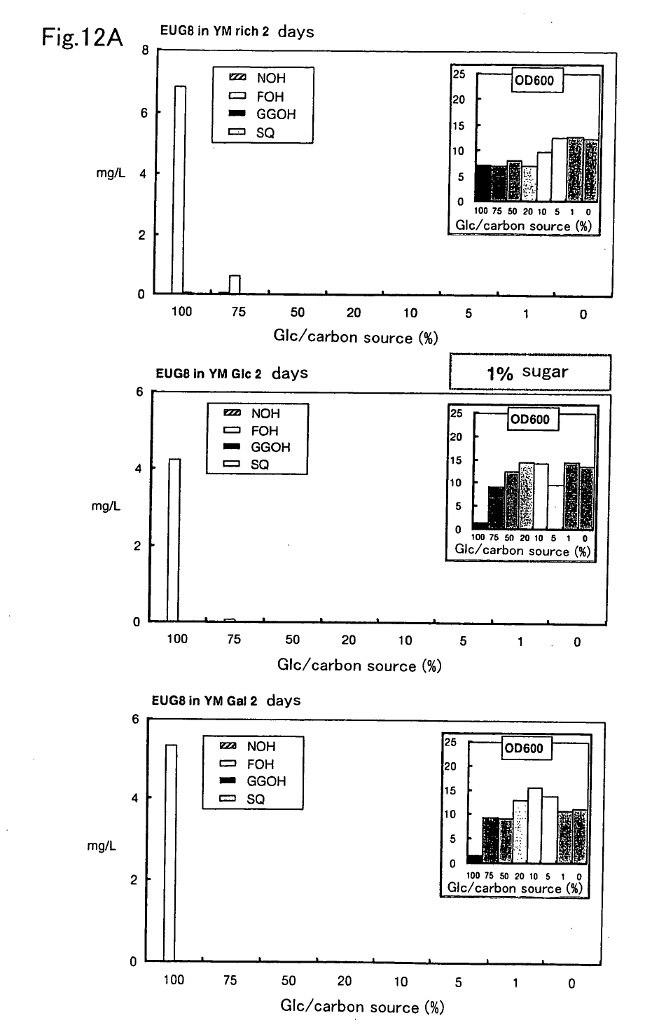

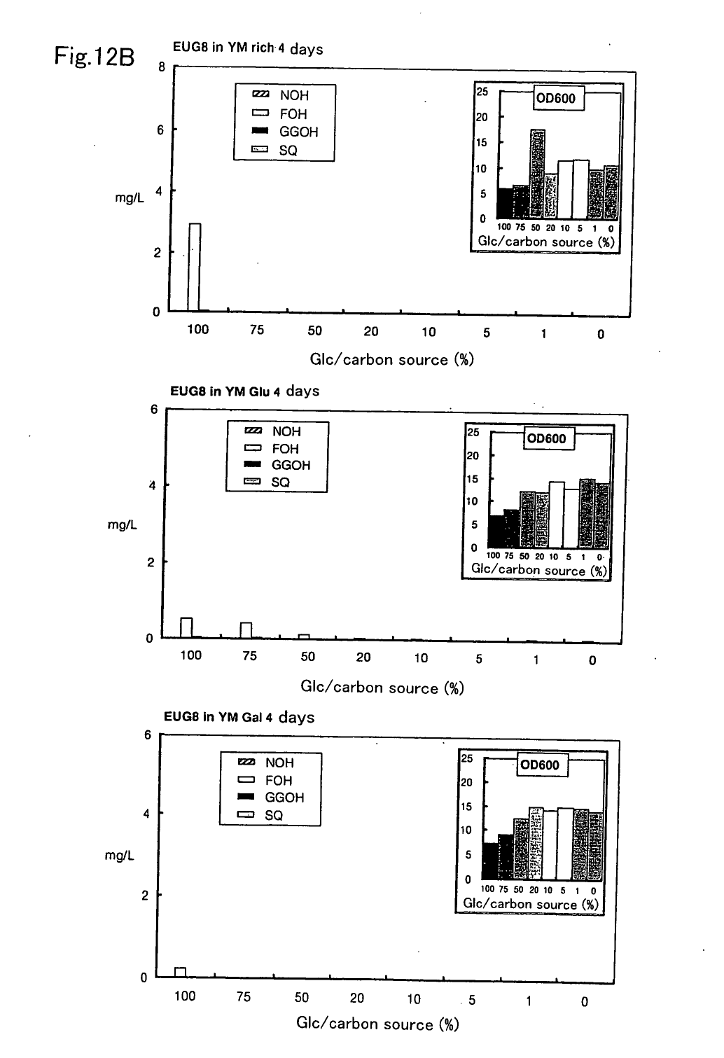

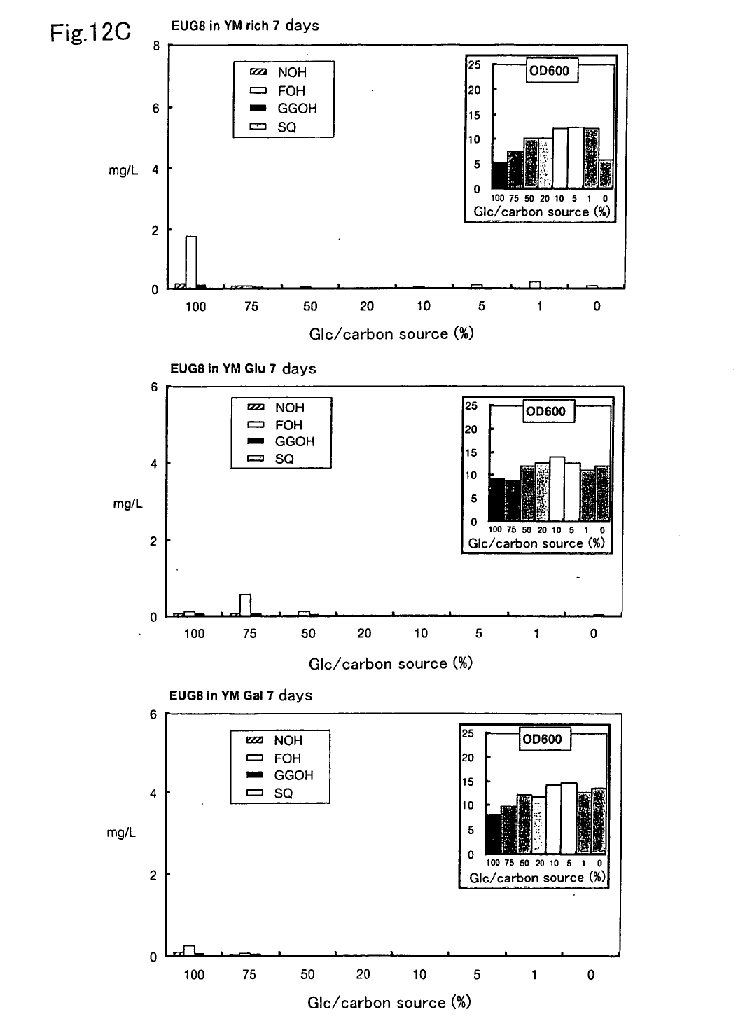

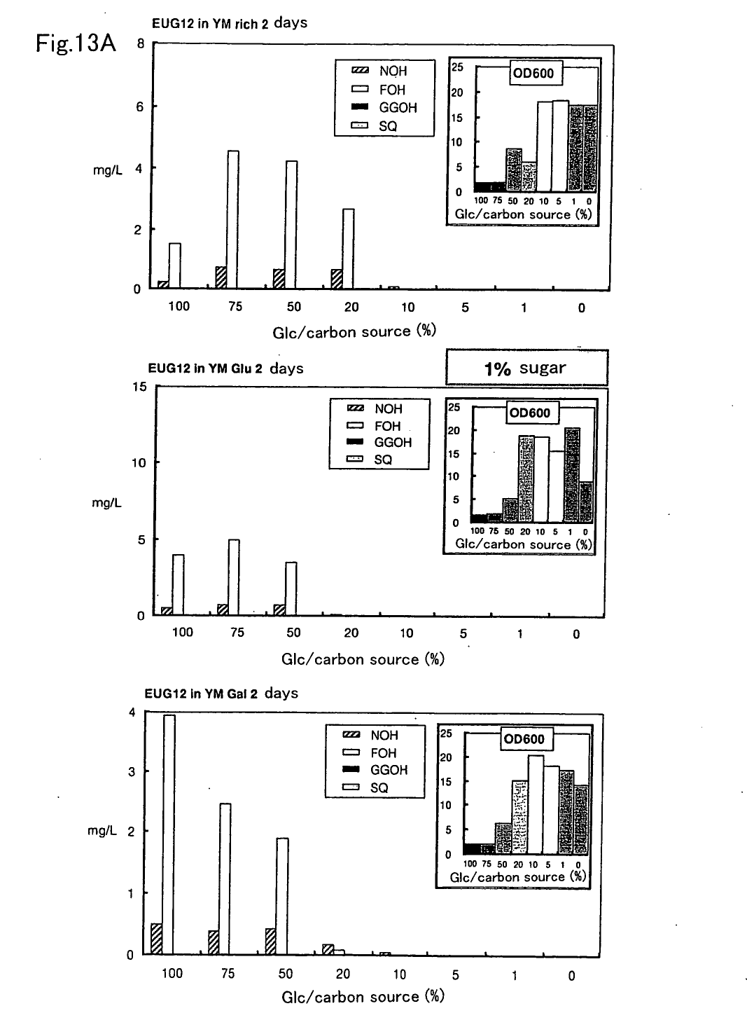

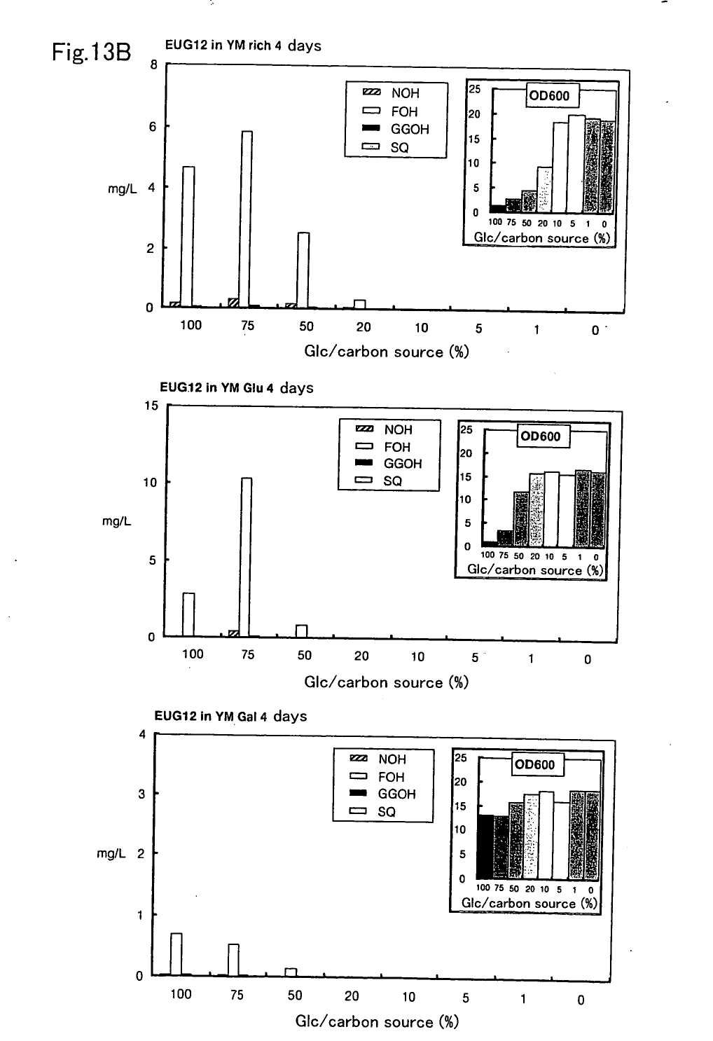

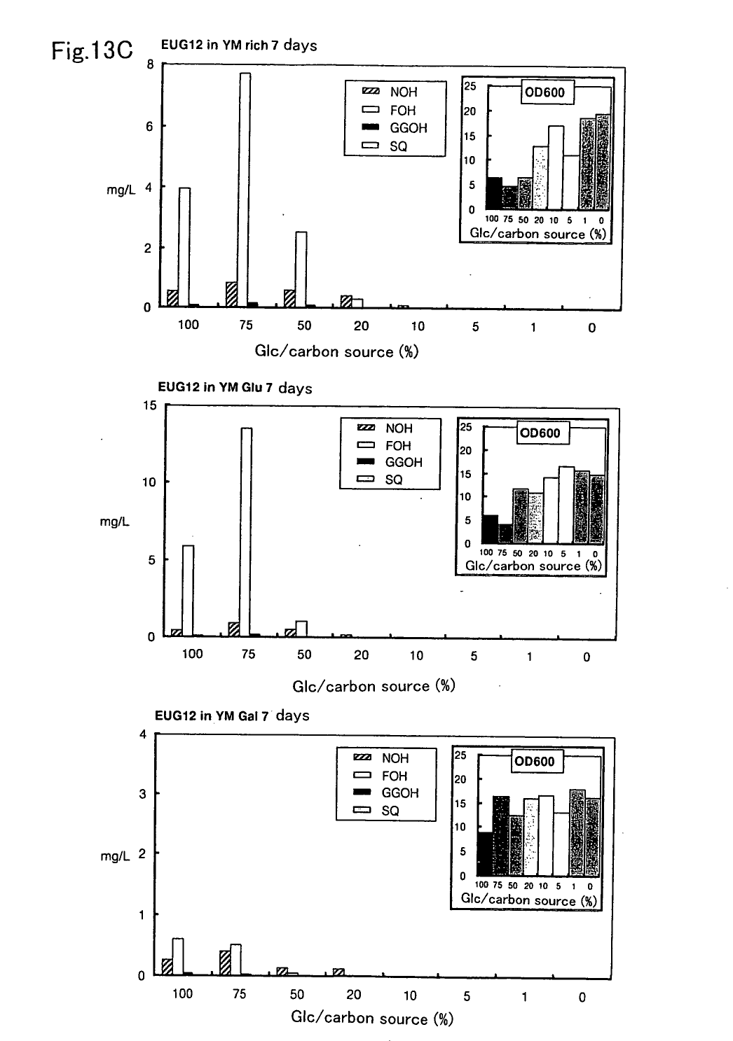

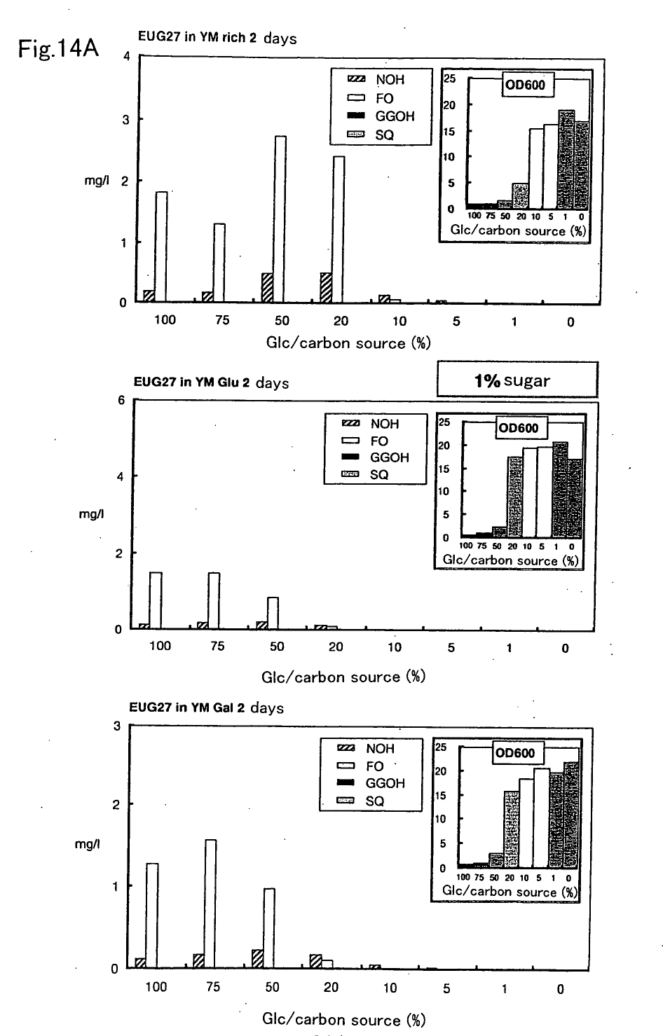

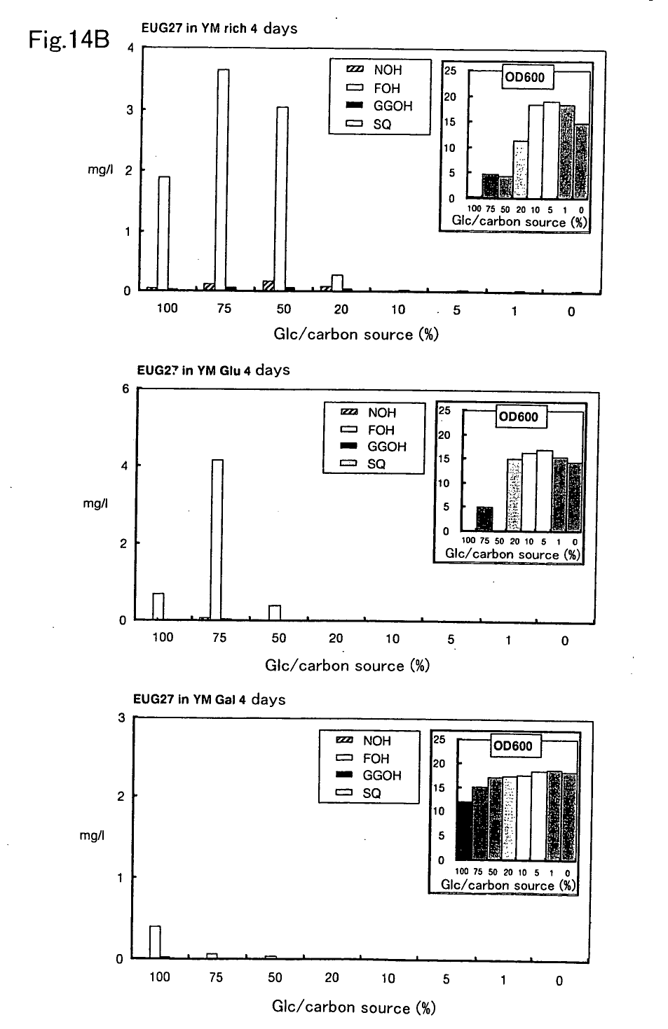

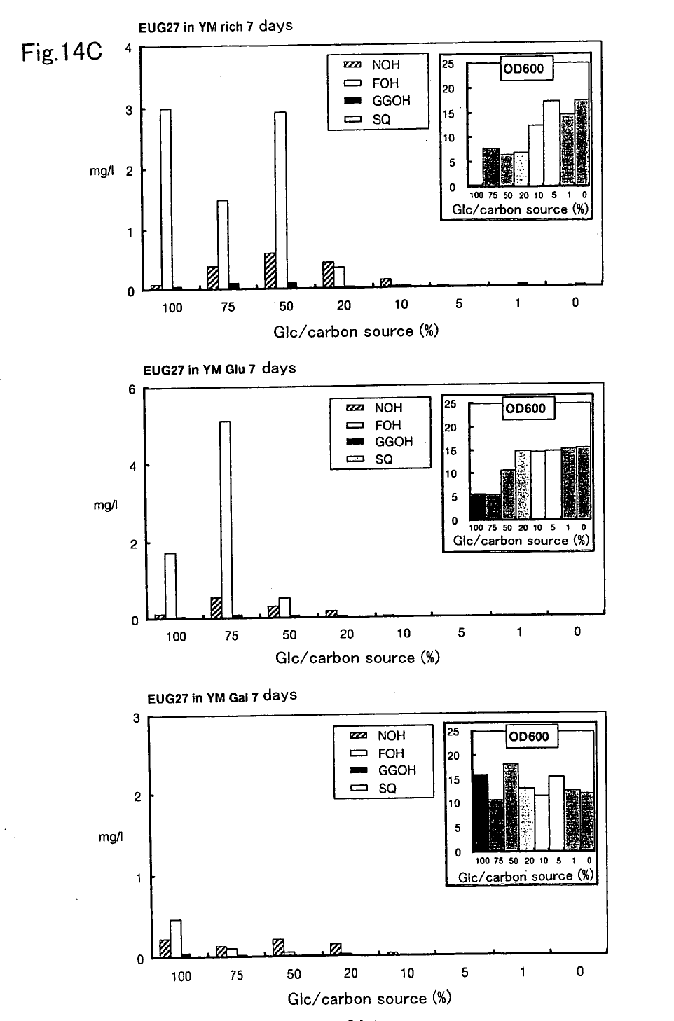

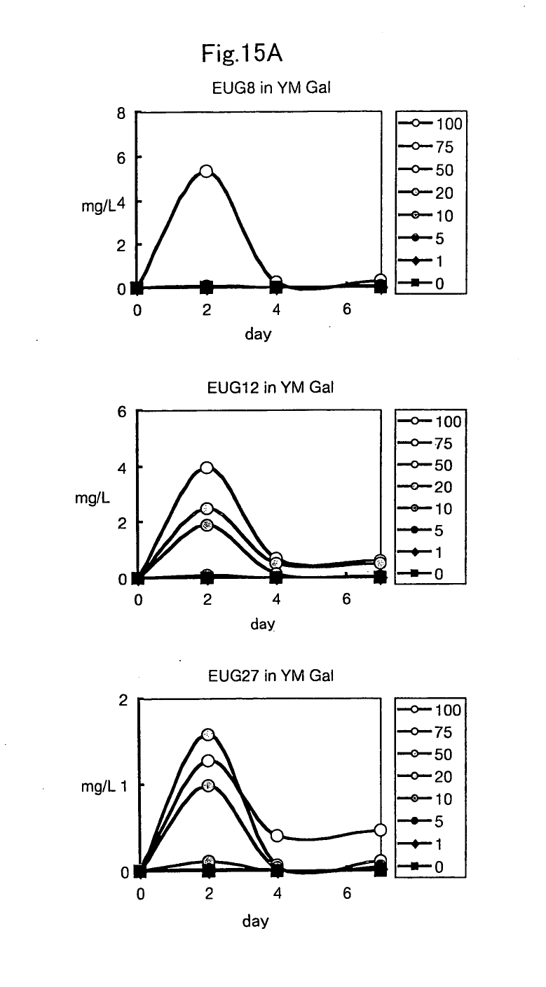

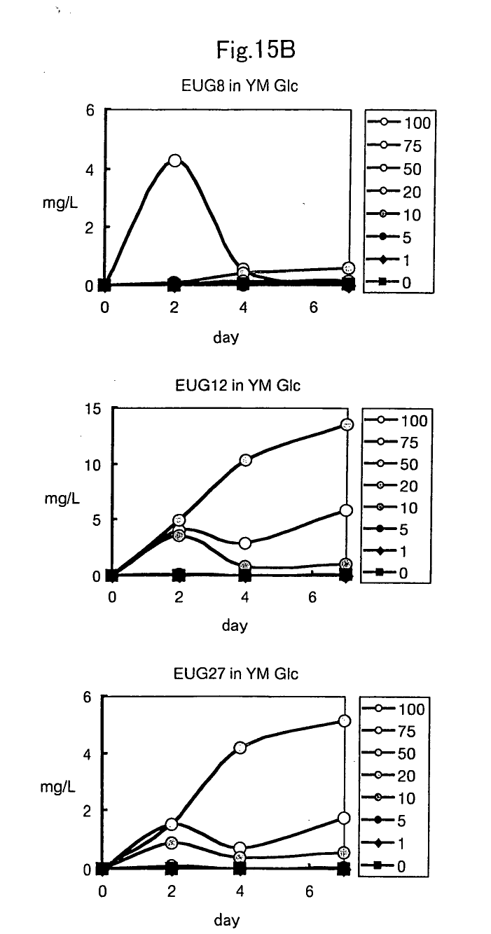

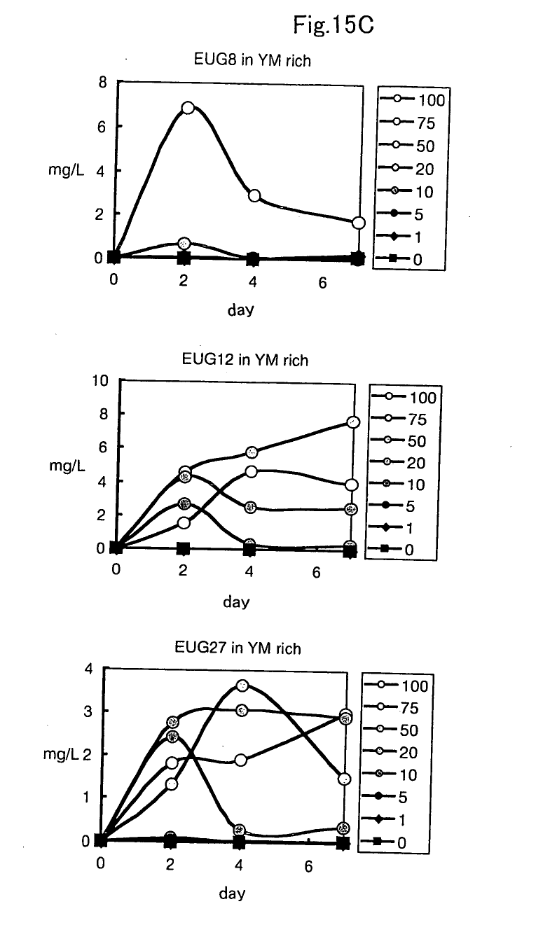

FIGS. 12 to 14 show the yields of prenyl alcohols when EUG8, EUG12 and EUG27 were cultured under varied Gic and Gal concentrations. Although FOH was the only product in most cases, it can be seen that production profile is different between A451-derived EUG8 (FIGS. 12A-C) and YPH-derived EUG12 (FIGS. 13A-C) and EUG27 (FIGS. 14A-C). EUG8 exhibited the highest yield when the sugar composition in the initial medium was 100% Glc under any of the conditions used. On the other hand, EUG12 and EUG27 exhibited good yields when the sugar composition in the initial medium was 75% Glc. Further, EUG12 and EUG27 in FOH well under such conditions that the sugar composition in the initial medium was 75% Glc (in 1% (w/v) total sugar) and that Glc was added further to the medium on day 2 to make the final sugar concentration 5% (w/v). It is believed that the transcription of ERG9 minimum to cell growth was carried out under the condition of 75% Glc in the sugar composition of the initial medium, leading to an appropriate balance between cell growth and FOH. When strains were cultured in YM Gal medium, a decrease in FOH production is observed on day 4 and thereafter. It is believed that such a decrease occurs because FOH in the medium is assimilated as the intracellular synthesis of Erg is activated as a result of the promotion of ERG9 transcription by the addition of Gal. The results of FOH production summarized in FIGS. 15A-C would help understanding of the difference in production profile between A451-derived EUG8 and YPH-derived EUG12 and EUG27. Under the “conditions that the sugar composition in the initial medium was 75% Glc (1% (w/v) sugar) and that Glc was added further to the medium on day 2 to make the final sugar concentration 5% (w/v)”, there is almost no change in OD₆₀₀ values. However, FOH yield increased from day 2 to day 4, and from day 4 to day 7 under such conditions. Accordingly, it is believed that these cells were in a physiological state where FOH is produced without cell division. If such cells can be₃₁ fixed, it will be possible to develop an efficient system for FOH production.

By the development of EUG strains described in this Example, it is possible to reduce the amount of squalene synthase gene transcript having translational activity. Different from squalene synthase gene-deficient strains (such as ATCC64031) obtained by introducing mutations such as substitution, insertion or deletion into the coding region of the gene, EUG strains are capable of producing FOH (an active prenyl alcohol) through fermentation in a common medium such as YM7 without introduction of special mechanisms to complement the lethality because of the squalene synthase deficiency (e.g., addition of sterol intake ability or Erg addition). Thus, it is possible to create a system for FOH production using EUG strains. Further, since EUG strains were created from conventional hosts for recombination, they can be genetically modified further easily (unlike strains such as ATCC64031) and are applicable to the development of strains with still higher productivity.

### EXAMPLE 3

### Construction of Expression Vectors

(1) E. coli-S. cerevisiae Shuttle Vectors

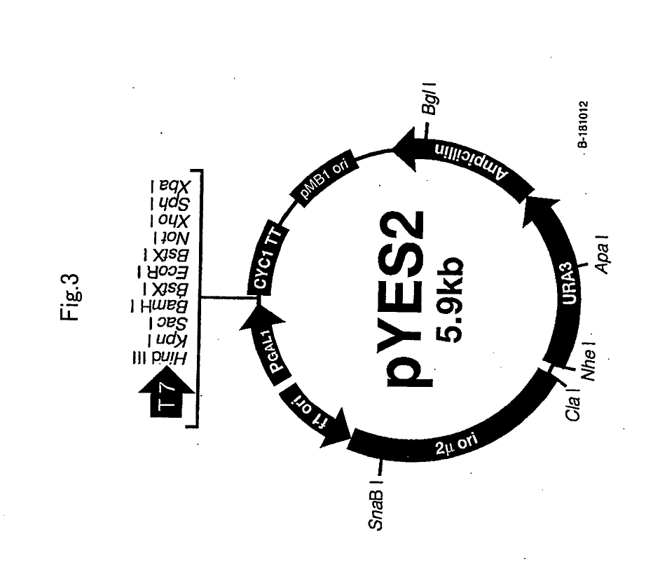

Plasmids pRS404 and pRS405 were purchased from Stratagene. Plasmid pYES2 (FIG. 3) was purchased from Invitrogen (Carlsbad, Calif.).

(2) Genomic DNA

Genomic DNA was prepared from S. cerevisiae YPH499 according to the protocol attached to Dr. GenTLE™, a genomic DNA preparation kit for yeast purchased from Takara.

Plasmid DNA from E. coli was prepared with Wizard PureFection Plasmid DNA Purification System purchased from Promega (Madison, Wis.).

(3) Insertion of CYC1t Fragment into pRS Vectors

CYC1t (CYC1 transcription terminator) fragment was prepared by PCR. The following oligo-DNAs, XhoI-Tcyc1FW and ApaI-Tcyc1RV, were used as PCR primers. As a template, pYES2 was used.

| | | |
|---|---|---|
| XhoI-Tcyc1FW: | 5′-TGC ATC TCG AGG GCC GCA TCA TGT AAT TAG-3′ | (SEQ ID NO:43) |
| ApaI-Tcyc1RV: | 5′-CAT TAG GGC CCG GCC GCA AAT TAA AGC CYf CG-3′ | (SEQ ID NO:44) |

Briefly, 50 μl of a reaction solution containing 0.1 μg of pYES2, 50 pmol of each primer DNA, 1×Pfu buffer containing MgSO₄ (Promega, Madison, Wis.), 10 mmol dNTPs, 1.5 units of Pfu DNA polymerase (Promega) and 1 μl of Perfect Match polymerase enhancer (Stratagene) was prepared. Following an initial denaturation of 2 min at 95° C., PCR was carried out for 30 cycles each consisting of denaturation of 45 sec at 95° C., annealing of 30 sec at 60° C., and extension of 1 min at 72° C. A final extension of 5 min at 72° C. was done. After completion of the reaction, the solution was stored at 4° C. The amplified two DNA fragments were digested with XhoI and ApaI, and subjected to agarose gel electrophoresis to purity a 260 bp DNA fragment, which was designated CYC1t-XA.

CYC1t-XA was inserted into the XhoI-ApaI site of pRS404 and pRS405 to thereby obtain pRS404Tcyc and pRS405Tcyc, respectively.

(4) Preparation of a Transcription Promoter

A DNA fragment comprising a transcription promoter was prepared by PCR using yeast genomic DNA as a template. The DNA primers used are as follows.

| | | |
|---|---|---|
| SacI-Ptdh3FW: | 5′-CAC GGA GCT CCA GTT CGA GTT TAT CAT TAT CAA-3′ | (SEQ ID NO:45) |
| SacII-Ptdh3RV: | 5′-CTC TCC GCG GTT TGT TTG TTT ATG TGT GTT TAT TC-3′ | (SEQ ID NO:46) |

As a reaction solution, a 100 μl solution containing 0.46 μg of yeast genomic DNA, 100 pmol of each primer DNA, 1×ExTaq buffer (Takara), 20 nmol dNTPs, 0.5 U of ExTaq DNA polymerase (Takara) and 1 μl of Perfect Match polymerase enhancer was prepared. Following an initial denaturation of 2 min at 95° C., PCR was carried out for 30 cycles each consisting of 45 sec at 95° C., 1 min at 60° C. and 2 min at 72° C. A final extension of 4 min at 72° C. was done. After completion of the reaction, the solution was stored at 4° C. The amplified DNA was digested with SacI and SacII, and subjected to agarose gel electrophoresis to purify a 0.7 kbp fragment. Thus, TDH3p was obtained.

(5) Preparation of 2 μl DNA Replication Origin Site

pYES2, which is a YEp vector, was digested with SspI and NheI. The resultant 1.5 kbp fragment containing 2μ DNA replication origin (2μ ori) was purified by agarose gel electrophoresis and then blunt-ended. This DNA fragment was designated 2μOriSN.

(6) Preparation of YEp Expression Vectors

2μOriSN was inserted into the Nael site of pRS404Tcyc and pRS405Tcyc pretreated with BAP (bacterial alkaline phosphatase: Takara). The resultant plasmids were transformed into E. coli SURE2, and then plasmid DNA was prepared. The plasmid DNA was digested with DraIII EcoRI, HpaI or PstI; and PvuII, followed by agarose gel electrophoresis to examine the insertion and the direction of 2μ ori. The resultant pRS404Tcyc and pRS405Tcyc into which 2μ ori had been inserted in the same direction as in pYES2 were designated pRS434Tcyc2μOri and pRS435Tcyc2μOri, respectively. The resultant pRS404Tcyc and pRS405Tcyc into which 2μ ori had been inserted in the opposite direction to that in pYES2 were designated pRS444Tcyc2μOri and pRS445Tcyc2μOri, respectively.

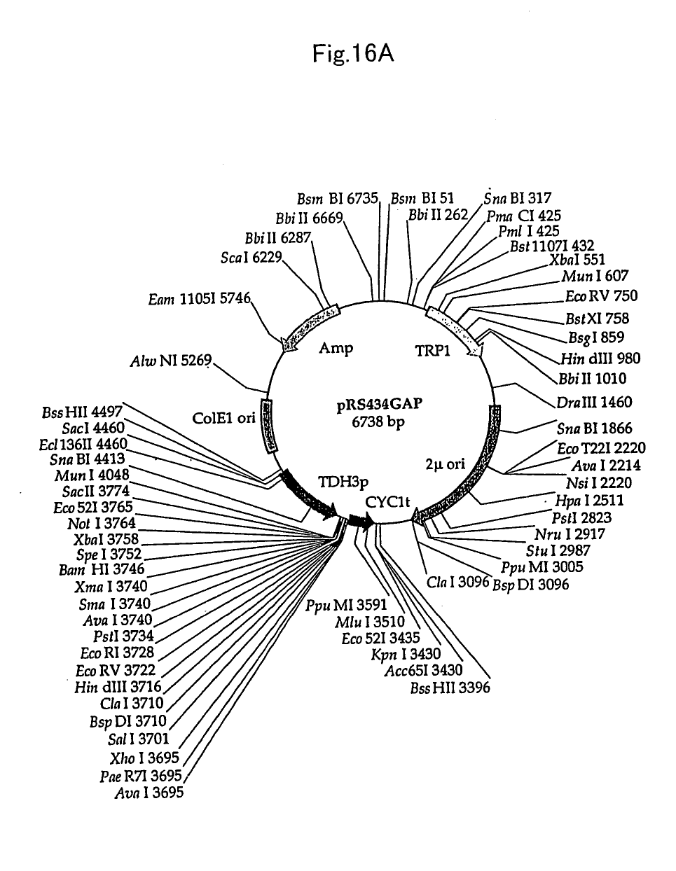

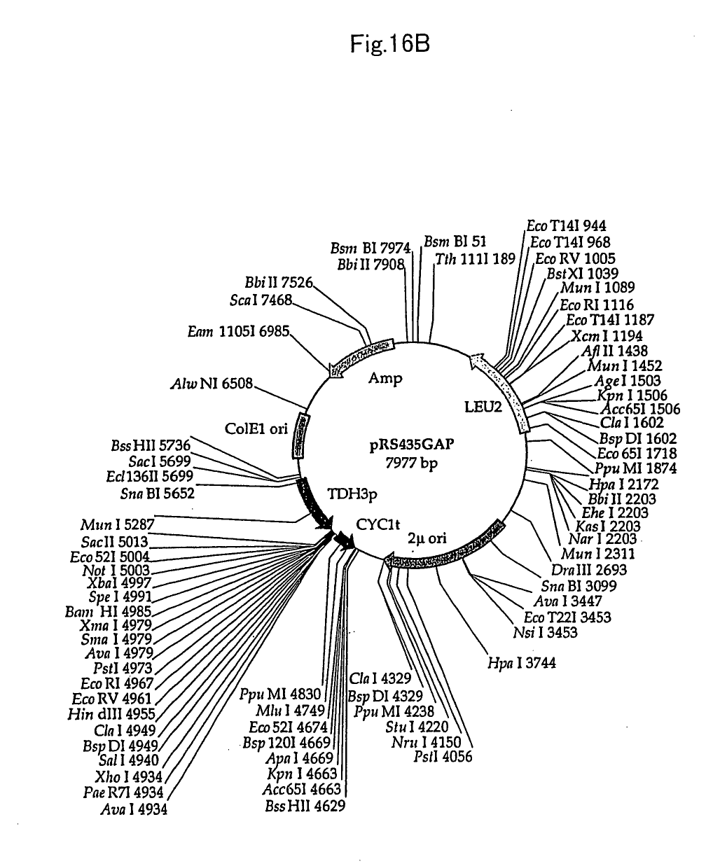

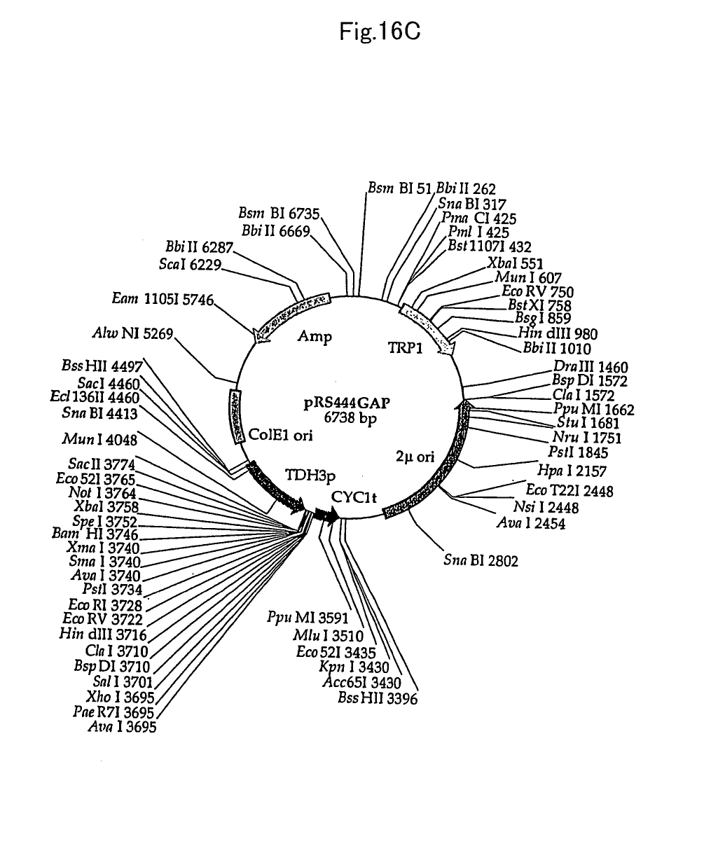

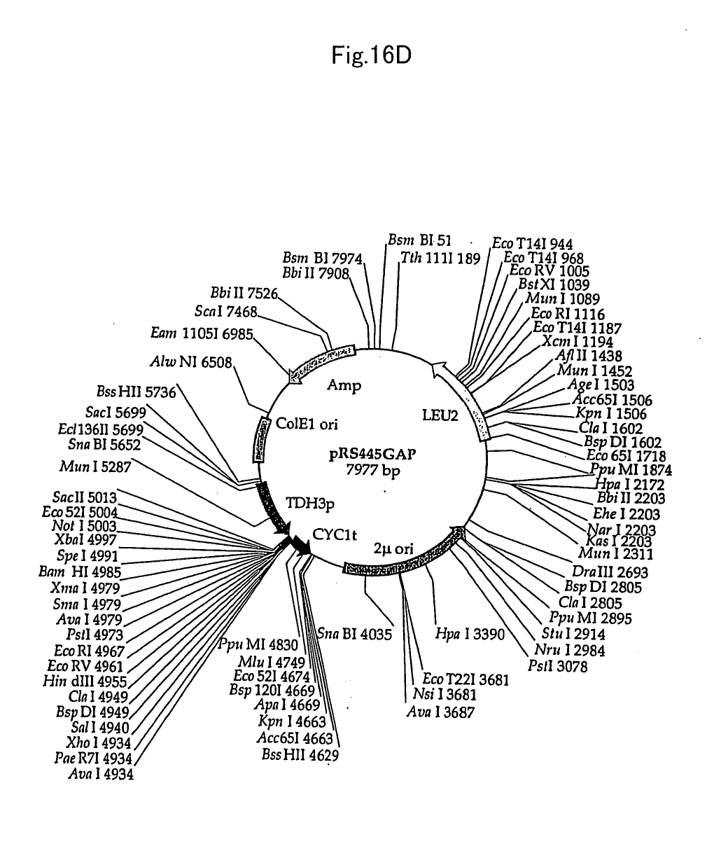

The transcription promoter-containing fragment, i.e., TDH3p (GAPp), was inserted into the SacI-SacII site of the above-described four plasmids pRS434Tcyc2μOri, pRS435Tcyc2μOri, pRS444Tcyc2μOri and pRS445Tcyc2μOri to clone the DNA. As a result, the following plasmids were obtained: (i) pRS434GAP from pRS434Tcyc2μOri; (ii) pRS435GAP from pRS435Tcyc2μOri; (iii) pRS444GAP from pRS444Tcyc2μOri; and (iv) pRS445GAP from pRS445Tcyc2μOri (FIGS. 16A-D).

(7) Introduction of YEp Expression Vectors into Yeast

Approximately 40 ng of each YEp expression vector was introduced into YPH499 using Frozen-EZ Yeast Transformation II kit (Zymo Research, Orange, Calif.). (The procedures followed the protocol attached to the kit.) Then, colonies growing on SD-W agar plate (DOB+CMS-Trp; BIO 101, Vista, Calif.) were examined. Since 1000 or more Try non-requiring colonies were observed, it was confirmed that the DNA replication site of each of the YEp expression vectors prepared functions and that each vector is retained normally in the recombinant as a plasmid.

### EXAMPLE 4

### Cloning of IPP Biosynthetic Pathway-Related Enzyme Genes

In the cloning of genes from yeast cDNA, an S. cerevisiae DBY746-derived cDNA library “Quick-Clone cDNA” purchased from Clontech (Palo Alto, Calif.) was used.

(1) Cloning of Farnesyl Diphosphate Synthase Gene

S. cerevisiae-derived FPP synthase gene ERG20:

A DNA fragment of approximately 0.9 kbp encoding S. cerevisiae FPP synthase gene ERG20 (SEQ ID NO: 1) was amplified by PCR (polymerase chain reaction) using the above cDNA as a template. The PCR primers used are as follows.

| | |
|---|---|
| Primer 1 (SCFPS1): | |
| 5′-ATG GCT TCA GAA AAA GAA ATT AG-3′ | (SEQ ID NO:47) |
| Primer 2 (SCFPS2): | |
| 5′-CTA TTT GCT TCT CTT GTA AAC TT-3′ | (SEQ ID NO:48) |

| | |
|---|---|
| 10 x ExTaq buffer (Takara) | 5 μl |
| 2.5 mM dNTP mix | 4 μl |
| 5 U/μl ExTaq (Takara) | 1 μl |
| 10 pmol Primer 1 | |
| 10 pmol Primer 2 | |
| 0.5 ng cDNA | |

To give a 50 μl solution in total

The PCR was carried out in the reaction solution described above for 30 cycles each consisting of 45 sec at 94° C., 1 min at 55° C. and 2 min at 72° C. Unless otherwise indicated, PCR reactions in the following Examples were carried out under similar conditions.

The amplified fragment was purified by agarose gel electrophoresis and then cloned into pT7Blue-T (Novagen, Madison, Wis.) by T/A ligation. It was found that ERG20 was inserted in the same direction as lacZ in pT7Blue-T. The nucleotide sequence of the cloned fragment was determined and compared to the corresponding nucleotide sequence registered in SGD. As a result, no PCR errors were found in nucleotide positions 1-300 and 610-1059. The amino acid sequence encoded by ERG20 is shown in SEQ ID NO: 2.

The plasmid DNA prepared was designated pT7ERG20.

(2) Cloning of Geranylgeranyl Diphosphate Synthase Gene

S. cerevisiae-derived GGPP synthase gene BTS1 (SEQ ID NO: 3) was cloned as described below.

Based on information about the S. cerevisiae-derived GGPP synthase gene registered at the GenBank (http://www.ncbi.nln.nih.gov/Genbank/index.html/) (Accession No. (A.N.): U31632) (Y. Jiang, et al., J. Biol. Chem. 270 (37), 21793-21799 (1995)), a pair of primers matching the N-terminal and C-terminal of the protein encoded by the gene were designed. Using these primers and a yeast cDNA library (Clontech; No. CL7220-1) as a template, PCR was carried out.

| | |
|---|---|
| N-terminal primer: | |
| 5′-ATG GAG GCC AAG ATA GAT GAG CT-3′ | (SEQ ID NO:49) |
| C-terminal primer: | |
| 5′-TCA CAA TTC GGA TAA GTG GTC TA-3′ | (SEQ ID NO:50) |

The PCR was performed using Perfect Match Polymerase Enhancer for 30 cycles each consisting of denaturation of 45 sec at 94° C., annealing of 1 min at 55° C. and extension of 2 min at 72° C.

A fragment of interest (approx. 1.0 kbp) was confirmed. This fragment (BTS1) was cloned into pT7Blue T vector capable of TA cloning, followed by sequencing of the entire region of BTS1. The results revealed that the nucleotide sequence of this gene was completely identical with the corresponding nucleotide sequence registered at the GenBank. Thus, it was confirmed that this gene is the S. cerevisiae-derived GGPP synthase gene. The amino acid sequence encoded by BTS1 is shown in SEQ ID NO: 4.

(3) Cloning of HMG-CoA Reductase Gene

The cloning of S. cerevisiae-derived HMG1 gene was carried out as described below.

Based on information about S. cerevisiae-derived HMG1 gene (A.N.: M22002) (M. E. Basson, et al., Mol. Cell. Biol. 8, 3797-3808 (1988): SEQ ID NO: 5) registered at the GenBank, a pair of primers were designed which match the N-terminal and the C-terminal of the protein encoded by this gene. Using these primers and the yeast cDNA library (Clontech) as a template, PCR was carried out.

| | |
|---|---|
| N-terminal primer: | |
| 5′-ATG CCG CCG CTA TTC AAG GGA CT-3′ | (SEQ ID NO:51) |
| C-terminal primer: | |
| 5′-TTA GGA TTT AAT GCA GGT GAC GG-3′ | (SEQ ID NO:52) |

The PCR was carried out using Perfect Match Polymerase Enhancer for 30 cycles each consisting of denaturation of 45 sec at 94° C., annealing of 1 min at 55° C. and extension of 2 min at 72° C.

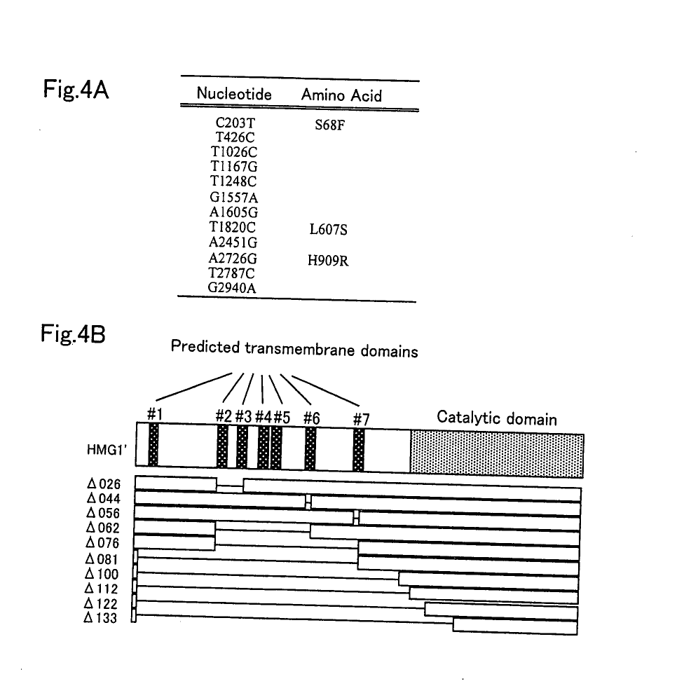

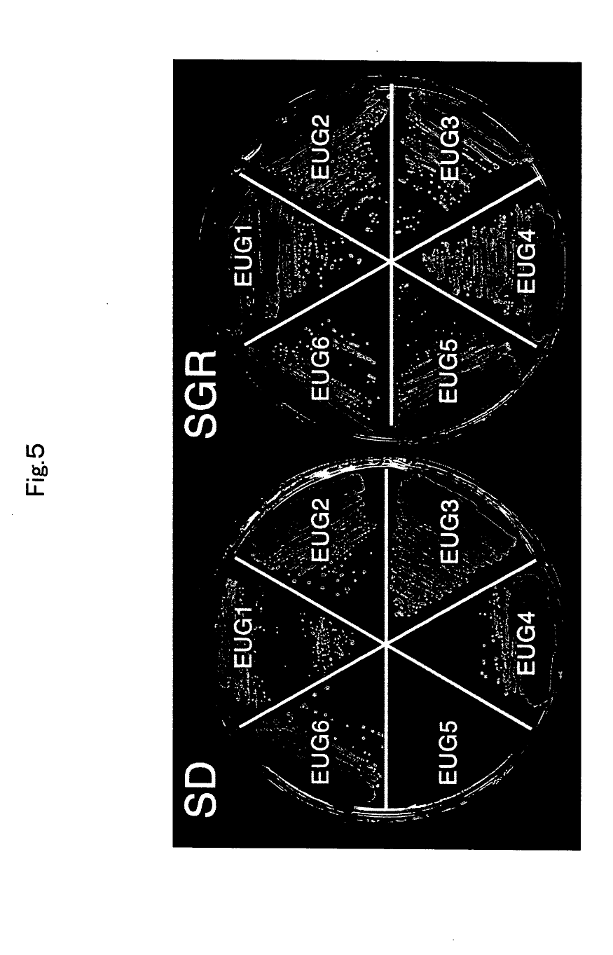

A fragment of interest (3.2 kbp) was confirmed. This fragment (HMG1) was cloned into pT7Blue T vector capable of TA cloning to thereby obtain pT7HMG1. The nucleotide sequence of the thus cloned HMG1 was determined. As a result, the nucleotide sequence as shown in SEQ ID NO: 5 and the amino acid sequence as shown in SEQ ID NO: 6 were confirmed. The thus determined nucleotide sequence was partially different from the corresponding nucleotide sequence registered at the GenBank due to PCR errors (FIG. 4A). This PCR error-containing, mutant type HMG-CoA reductase gene is designated HMG1′.

(4) Correction of PCR Errors in HMG-CoA Reductase Gene

PCR errors were corrected by subcloning the HMG1 fragment from pT7HMG1 and correcting those mutations resulting from PCR errors in the HMG1 coding region that would cause amino acid substitutions.

Briefly, the HMG1′ fragment was subcloned from plasmid pT7HMG1 complising HMG1′, a PCR error type mutant of HMG-CoA reductase. Then, the amino acid substitutions resulting from the PCR errors in the coding region of HMG1 were corrected by site-directed mutagenesis to thereby prepare pALHMG106. The details of this preparation are as described below.

Plasmid pT7HMG1 was used as cloned HMG1. As a vector for introducing site-directed mutations, pALTER-1 (Promega) was purchased.

Site-directed mutagenesis was carried out according to the procedures described in “Protocols and Application Guide, 3rd edition, 1996 Promega, ISBN 1-882274-57-1” published by Promega. As oligos for introducing mutations, the following three oligos were synthesized chemically.

| | |
|---|---|
| HMG1 (190-216): | |
| 5′-CCAAATAAAGACTCCAACACTCTATTT-3′ | (SEQ ID NO:53) |
| HMG1 (1807-1833): | |
| 5′-GAATTAGAAGCATTATTAAGTAGTGGA-3′ | (SEQ ID NO:54) |
| HMG1 (2713-2739): | |
| 5′-GGATTTAACGCACATGCAGCTAATTTA-3′ | (SEQ ID NO:55) |

First, pT7HMG1 was digested with SmaI, ApaLI and SalI, followed by preparation of a 3.2 kbp HMG1 fragment by agarose gel electrophoresis. This fragment was inserted into the SmaI-SalI site of pALTER-1 to prepare pALHMG1. After denaturation of this plasmid with alkali, the above-described oligos for introducing mutations, Amp repair oligo (Promega) as repair oligos, and Tet knockout oligo (Promega) as knockout oligos were annealed thereto. The resultant plasmid was introduced into E. coli ES1301 (Promega). Transformants retaining those plasmids into which site-directed mutations had been introduced were selected and cultured with 125 μg/ml of ampicillin to prepare plasmid DNA. The nucleotide sequence of the resultant plasmid DNA was examined with primers having the sequences as shown below. The results revealed that all the sequences corresponding to HMG1 (190-216), FIMG1 (1807-1833) and HMG1 (2713-2739) were corrected so that they had the sequences of these oligonucleotides (SEQ ID NO: 13). The amino acid sequence encoded by the corrected nucleotide sequence (SEQ ID NO: 14) was consistent with the amino acid sequence encoded by HMG1′ (SEQ ID NO: 12) (silent mutations).

| | |
|---|---|
| HMG1 (558-532): | |
| 5′-GTCTGCTTGGGTTACATTTTCTGAAAA-3′ | (SEQ ID NO: 56) |
| HMG1 (1573-1599): | |
| 5′-CATACCAGTTATACTGCAGACCAATTG-3′ | (SEQ ID NO: 57) |
| HMG1 (2458-2484): | |
| 5′-GAATACTCATTAAAGCAAATGGTAGAA-3′ | (SEQ ID NO: 58) |

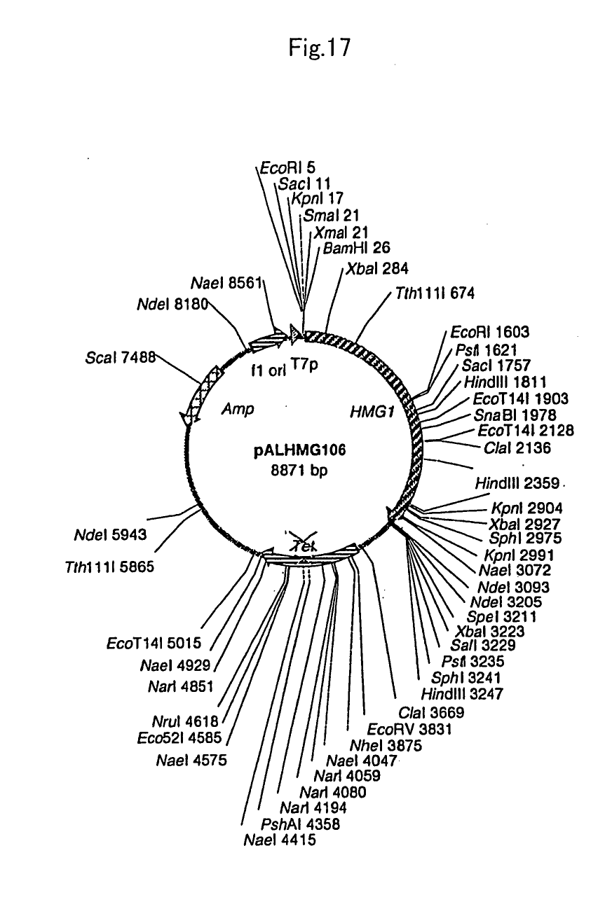

The plasmid where the sequence of the HMG1 coding region had been corrected was designated pALHMG106 (FIG. 17).

(5) Cloning of Diphosphomevalonate Decarboxylase Gene

A fragment of approx. 1.2 kbp encoding S. cerevisiae-derived diphosphomevalonate decarboxylase gene ERG19 (MVD1) (SEQ ID NO: 7) was amplified by PCR using cDNA as a template. The PCR primers used are as follows.

| | | |
|---|---|---|
| Primer 1 (SCU-1): | 5′-AAC TGC AGA TGA CCG TTT ACA CAG CAT CCG T-3′ | (SEQ ID NO: 59) |
| Primer 2 (SCU-2): | 5′-CGG AAT TCT TAT TCC TTT GGT AGA CCA GTC T-3′ | (SEQ ID NO: 60) |

(Restriction enzyme recognition sites are underlined.)

The PCR fragment was digested with PstI and EcoRI, purified by agarose gel electrophoresis, and cloned into the PstI-EcoRI site of pT7Blue. By these procedures, ERG19 (MVD1) was inserted into pT7Blue in the direction opposite to that of lacZ in this plasmid. The nucleotide sequence of the cloned fragment was determined and compared to the corresponding sequence registered in SGD. As a result, no PCR error was found. The amino acid sequence encoded by ERG19 is shown in SEQ ID NO: 8.

The plasmid DNA prepared was designated pT7ERG19.

### EXAMPLE 5

### Cloning of Mutant Genes

Cloning of Deletion Mutants of HMG-CoA Reductase Gene:

pT7HMG1 prepared in (3) in Example 4 was digested with BamHI, SalI and ScaI to obtain the HMG1′ gene having PCR errors. This gene was introduced into the BamHI-XhoI site of pYES2 (Invitrogen, Carlsbad, Calif.) to thereby obtain a recombinant vector pYES-HMG1. The nucleotide sequence within the vector was confirmed to be the nucleotide sequence of SEQ ID NO: 9. pYES is a shuttle vector for expression in yeast having the ori of yeast 2 μmDNA as a replication origin and GAL1 promoter inducible by galactose (FIG. 3).

In order to prepare deletion mutants of HMG-CoA reductase gene having deletion of regions corresponding to transmembrane domains, PCR was carried out using the vector pYES-HMG1 prepared above as a template to thereby generate DNA fragments in which a part of the HMG1 coding region is deleted. The resultant fragment was blunt-ended with Klenow enzyme, circularized by self-ligation, and transformed into E. coli JM109. Then, plasmid DNA was prepared. Synthetic DNA sequences used as primers and their combinations are shown in Table 1.

For each of the plasmid DNA obtained, it was confirmed with 373A DNA sequencer (Perkin Elmer, Foster City, Calif.) that there was no shift in the reading frame of amino acids between the upstream and downstream of the deleted region in HMG1, and that there was no amino acid substitution resulting from PCR errors around the junction site. As a result, the following plasmids were obtained which have no amino acid substitution resulting from PCR errors around the junction site and in which a part of the gene could be deleted without any shift in the reading frame. Deletion mutants of HMG1 gene are expressed as, e.g., “Δ02y” according to the deletion pattern (where y represents a working number that may be any figure), and a pYES2 vector comprising Δ02y is expressed as, e.g., pYHMG026. (This expression method is also applied to other deletion mutants.)

HMG1Δ026: SEQ ID NO: 15

HMG1Δ044: SEQ ID NO: 16

HMG1Δ056: SEQ ID NO: 17

HMG1Δ062: SEQ ID NO: 18

HMG1Δ076: SEQ ID NO: 19

HMG1Δ081: SEQ ID NO: 20

HMG1Δ100: SEQ ID NO: 21

HMG1Δ112: SEQ ID NO: 22

HMG1Δ122: SEQ ID NO: 23

HMG1Δ133: SEQ ID NO: 24

Plasmids: pYHMG026, pYHMG027, pYHMG044, pYHMG045, pYHMG059, pYHMG062, pYHMG063, pYHMG065, pYHMG076, pYHMG081, pYHMG083, pYHMG085, pYHMG094, pYHMG100, pYHMG106, pYHMG107, pYHMG108, pYHMG109, pYHMG112, pYHMG122, pYHMG123, pYHMG125, pYHMG133 and pYHMG134

### EXAMPLE 6

### Subcloning of Genes

In this Example, pRS vectors prepared in Example 3 were used that are E. coli-S. cerevisiae YEp shuttle vectors having a constitutive promoter.

(1) Subcloning of FPP Synthase Gene

S. cerevisiae-Derived FPP Synthase Gene ERG20:

pT7ER20 described in (1) in Example 4 was digested with XbaI and BamHI, and subjected to agarose gel electrophoresis to thereby purify a 1.1 kbp fragment of ERG20. This fragment was inserted into the XbaI-BamHI site of pRS435GAP and pRS445GAP to obtain pRS435GAP-ERG20 and pRS445GAP-ERG20, respectively.

(2) Subloning of GGPP Synthase Gene or Mutants Thereof

S. cerevisiae-Derived GGPP Synthase Gene BTS1:

The pT7Blue T vector described in (2) in Example 4 was digested with BamHI and SalI to obtain BTS1 fragment, which was.then introduced into the BamHI-XhoI site of pYES2 (Invitrogen). The resultant recombinant vector was designated pYESGGPS. pYESGGPS was digested with BamHI and MluI, and subjected to agarose gel

electrophoresis to purify a 1.3 kbp fragment. This fragment was inserted into the BamHI-MluI site of pRS435GAP and pRS445GAP to obtain pRS435GAP-BTS1 and pRS445GAP-BTS1, respectively.

(3) Subcloning of HMG-CoA Reductase Gene or Mutants Thereof

pALHMG106 (FIG. 17) described in (4) in Example 4 was digested with SmaI and SalI, and subjected to agarose gel electrophoresis to purify a 3.2 kbp fragment encoding the PCR error-corrected HMG1 gene. This fragment was inserted into the SmaI-SalI site of pRS434GAP and pRS444GAP. Physical maps of the HMG1-subcloned plasmids were examined by restriction enzyme mapping using XhoI, SpeI, NaeI and SphI, and by confirming the nucleotide sequences of the border regions of the inserted 3.2 kbp HMG1 gene. Then, those plasmids created exactly as designed were selected and designated pRS434GAP-HMG1 and pRS444GAP-HMG1, respectively.

Deletion mutants of HMG-CoA reductase gene were obtained from pYES2 plasmids incorporating deletion mutants of HMG1 (see Example 5) and cloned into pRS434GAP in the same manner as described in the preceding paragraph.

(4) Subcloning of Diphosphomevalonate Decarboxylase Gene

pT7ERG19 described in (5) in Example 4 was digested with BamHI and SalI, and subjected to agarose gel electrophoresis to purify an ERG19 fragment (BamHI-SalI 1.5 kbp). This fragment was inserted into the BamHI-SalI site of pRS435GAP and pRS445GAP. The ERG19-subcloned plasmids were examined by XbaI recognition site mapping, followed by selection of those plasmids created exactly as designed. Selected plasmids were designated pRS435GAP-ERG19 and pRS445GAP-ERG19, respectively.

### EXAMPLE 7

### Prenyl Alcohol Production by Gene-Transferred EUG Strains

(1) Strains Used

EUG5, EUG8, EUG12, EUG24, EUG27, EUG36 and EUG64 were used.

(2) Expression Plasmids

As diphosphomevalonate decarboxylase gene (ERG19) expression plasmid, pRS445GAP-ERG19 was used.

As HMG-CoA reductase gene (HMG1) expression plasmid, pRS434GAP-HMG1 and pRS444GAP-HMG1 were used. As deletion mutant type HMG1 expression plasmid, pRS434GAP-HMG026, pRS434GAP-HMG044, pRS434GAP-HMG056, pRS434GAP-HMG062, pRS434GAP-HMG076, pRS434GAP-HMG081, pRS434GAP-HMG100, pRS434GAP-HMG112, pRS434GAP-HMG122 and pRS434GAP-HMG133 were used. The numbers following “HMG” in these designations indicate deletion patterns (FIG. 4B). The deletion domains were deleted by PCR using the primer DNAs shown in Table 1.

As FPP synthase gene (ERG20) expression plasmid, pRS435GAP-ERG20 and pRS445GAP-ERG20 were used.

As GGPP synthase gene (BTS1) expression plasmid, pRS435GAP-BTS1 and pRS445GAP-BTS1 were used.

(3) Transformation

Transformation of the above-mentioned yeast strains was carried out using Frozen EZ yeast transformation II kit purchased from Zymo Research (Orange, Calif.) II kit. Transformants were grown on selection medium agar plates that are based on SGR medium and have appropriate auxotrophy as an indicator. For cloning, cultivation on selection medium agar plates was carried out twice.

(4) Cultivation

Transformants prepared were precultured in SGR selection medium. Then, 0.01-0.05 ml of each preculture broth was added to 1-5 ml of YM7, and cultured in a test tube 18 mm in diameter at 30° C. under reciprocal shaking at 130 rpm.

(5) Determination of Prenyl Alcohol Yields

An equal volume of methanol was added to the resultant culture broth and mixed. Approximately 2 volumes of pentane was added to this mixture, agitated vigorously and then left stationary. The resultant pentane layer was transferred into a fresh glass tube, which was then placed in a draft. Pentane was evaporated therein to condense the solute components. Subsequently, prenyl alcohols were identified and quantitatively determined by GC/MS. At that time, the degree of cell growth was also examined by diluting 50 μl of the culture broth 30-fold with water and measuring the absorbance at 600 nm. The yields of prenyl alcohols were determined with PH6890/5973 GC/MS system in the same manner as described in (1-2) in Example 2.

(6) Results and Considerations

(6-1) Prenyl Alcohol Production by ERG19 Expression Strains

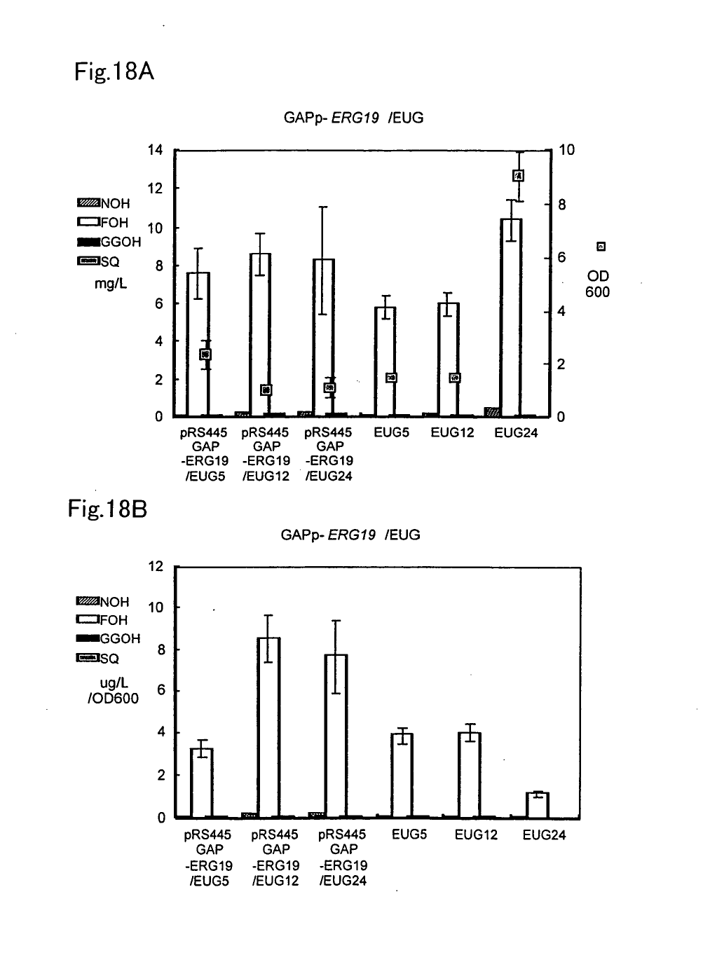

First, diphosphomevalonate decarboxylase gene ERG19 expression plasmid was introduced into EUG strains. pRS445GAP-ERG19 was introduced into A451-derived EUG5, YPH499-derived EUG12 and YPH500-derived EUG24, followed by determination of the yields of prenyl alcohols (FIG. 18A). Although it appears that FOH production is not significantly improved in any of the recombinant strains tested, FOH production efficiency is increased 2- to several-fold in the recombinants created from YPH-derived EUG12 and EUG24 when their FOH yields per OD₆₀₀ value of the culture broth are compared (FIG. 18B). Thus, it was demonstrated that, if a mutant cell of the invention capable of reducing an amount of squalene synthase gene transcript having translational activity without complete or partial deletion of its squalene synthase gene is used as a host, it can produce FOH efficiently by enhancement of ERG19 expression even in such environments where the number of cells is small.

(6-2) Prenyl Alcohol Production by HMG1 or HMG1Δ Expression Strains

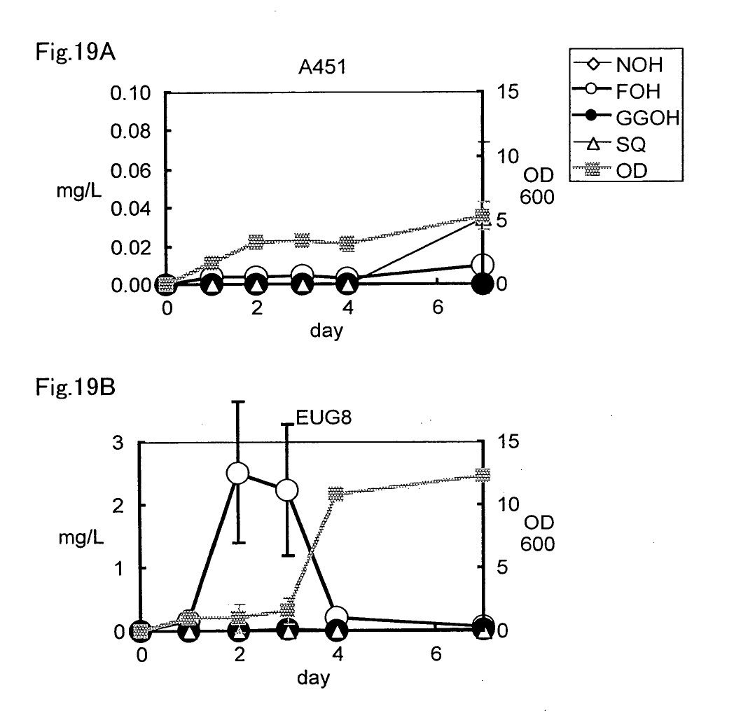

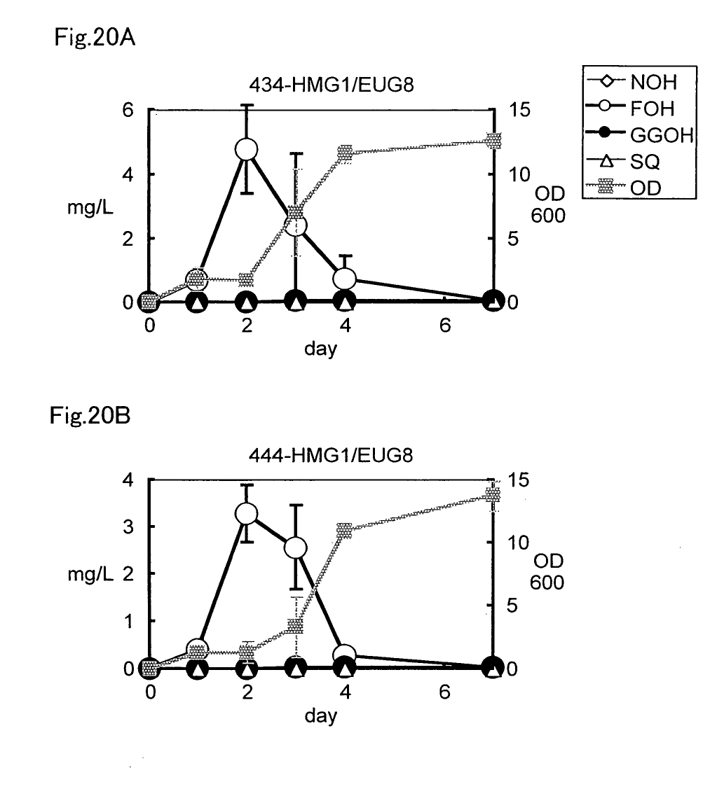

First, the prenyl alcohol yields of each 5 colonies of A451 and A451-derived EUG8 are shown in FIG. 19A and FIG. 19B, respectively. EUG8, the host, produced 2.5 mg/L of FOH on the average (3.3 mg/L at the maximum) when cultured for 2 days. Each 5 colonies of pRS434GAP-HMG1/EUG8 (FIG. 20A) and pRS444GAP-HMG1/EUG8 (FIG. 20B) were cultured, and mean values of their prenyl alcohol yields and standard deviations were recorded in graphs. They produced FOH at 4.8 mg/L on the average (6.7 mg/L at the maximum) and at 3.5 mg/L on the average (4.0 mg/L at the maximum) when cultured for 2 days, respectively. It was found that when EUG8 is used as a host, the improvement effect on prenyl alcohol productivity by the expression of HMG1 gene is more excellent than when A451 is used as a host.

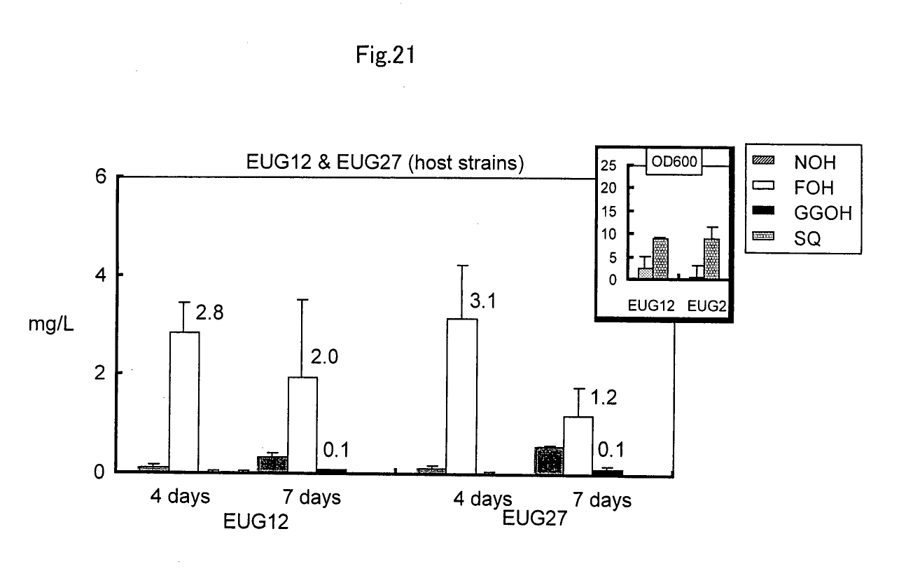

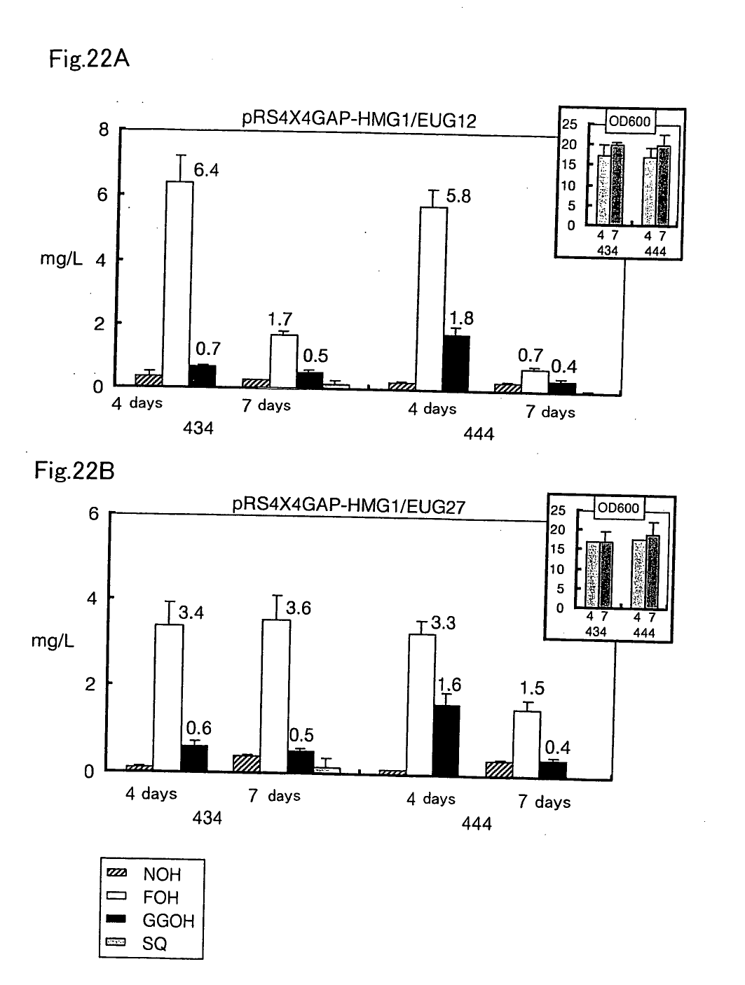

Subsequently, the effect of enhancement of HMG1 gene expression upon YPH499-derived EUG12 and YPH500-derived EUG27 was examined. As already reported in Japanese Unexamined Patent Publication 5-192184 and by Donald et al., (1997) Appl. Environ. Microbiol. 63, 3341-3344, HMG1-transferred YPH499 or YPH500 only shows an increase in SQ production but produces little prenyl alcohols. These facts were confirmed by preliminary experiments. Each 5 colonies of EUG12 and EUG27 were cultured in liquid medium, and the prenyl alcohol accumulation was determined on day 4 and day 7 of the cultivation. The results are shown in FIG. 21. The major product was FOH; NOH (which is an isomer of FOH) was produced in small amounts and GGOH was produced little. When HMG1 expression plasmid (pRS434GAP-HMG1 or pRS444GAP-HMG1) was introduced into EUG12 and EUG27, the results shown in FIG. 22 were obtained. It was remarkable that the number of cells increased as a result of partial complementarion of the erg9 phenotype by the enhancement of HMG1 expression. With respect to FOH production, the yield increased almost 2-fold in recombinants from EUG12 (FIG. 22A), whereas the yield remained on the same level in recombinants from EUG27 was used (FIG. 22B). On the other hand, GGOH production was improved in both recombinants; 2.05 mg/L of GGOH was accumulated in both of them at the maximum. With respect to GGOH production, pRS444GAP-HMG1-transferred recombinants exhibited higher effect. Thus, it was demonstrated that HMG1 gene is effective not only on FOH production but also on GGOH production when EUG12 or EUG27 is used as a host. However, the tendency that production decreases when cultured for 7 days is characteristic to EUG strains.

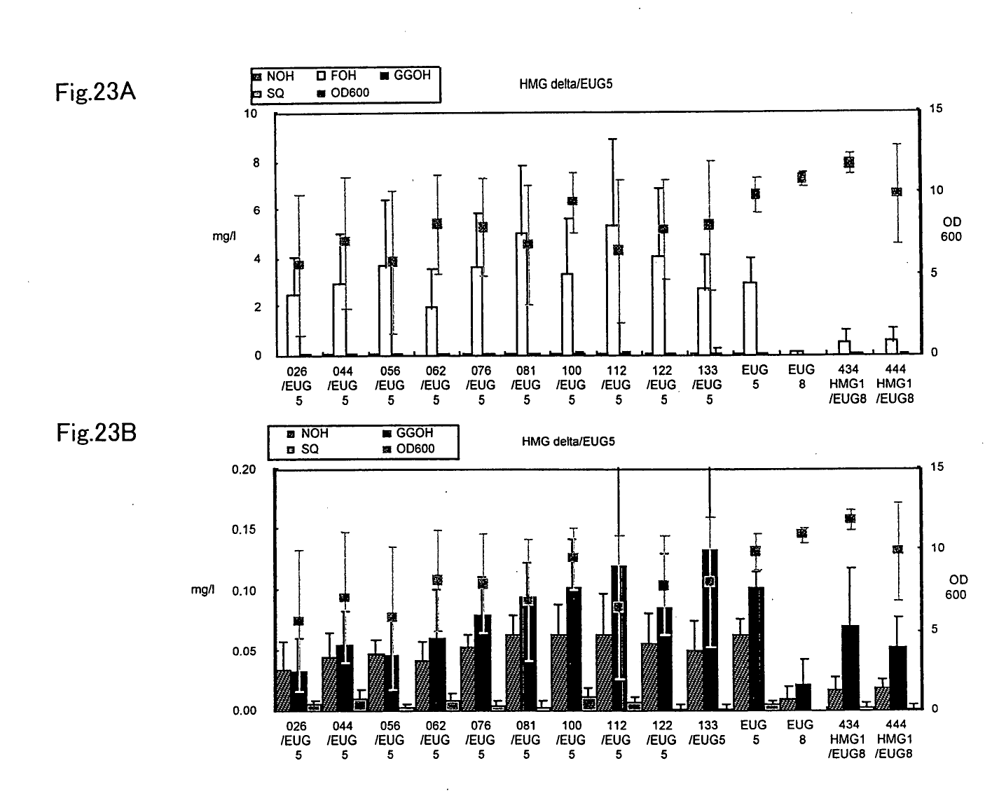

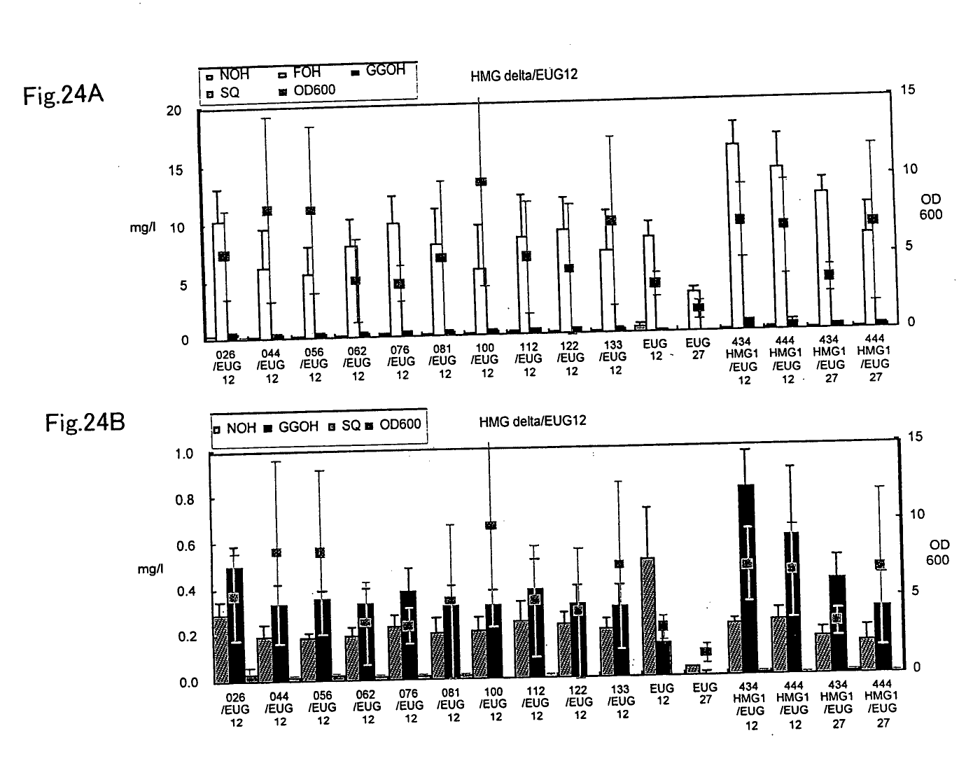

FIGS. 23-24 show the results of expression of individual deletion mutants of HMG1 in EUG5 and EUG12. When EUG5 was used, improvement of productivity as a result of the expression enhancement of those deletion mutants was not so remarkable. When EUG12 was used, the expression of those deletion mutants was effective in improving GGOH productivity, though their effects were smaller than the full-length HMG1 (FIG. 24B).

(6-3) Prenyl Alcohol Production by ERG20 Expression Strains

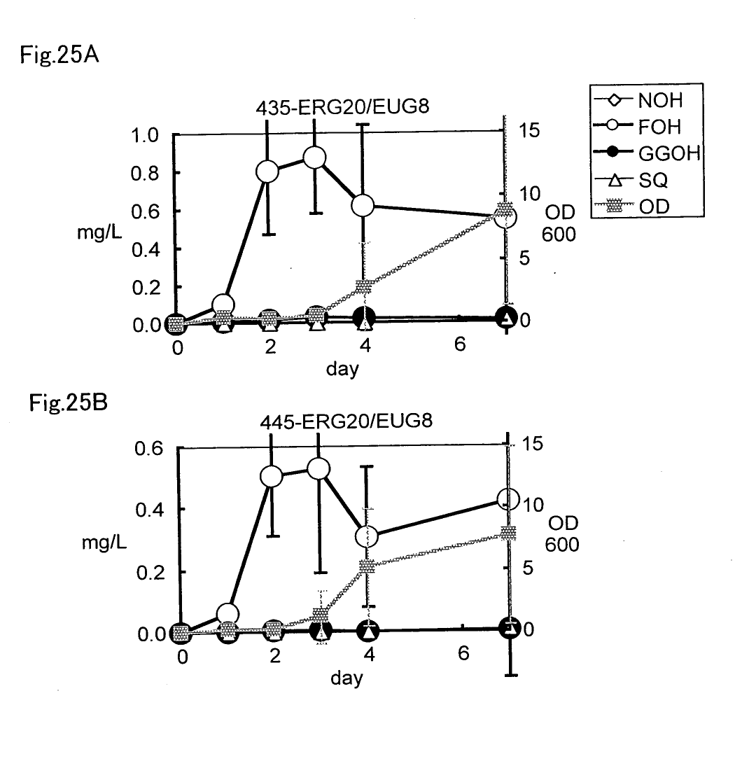

When each 5 colonies of pRS435GAP-ERG20/EUG8 (FIG. 25A) and pRS445GAP-ERG20/EUG8 (FIG. 25B) were cultured, the average FOH yields were 0.9 mg/L and 0.5 mg/L, respectively, and the maximum yield was 1.5 mg/L.

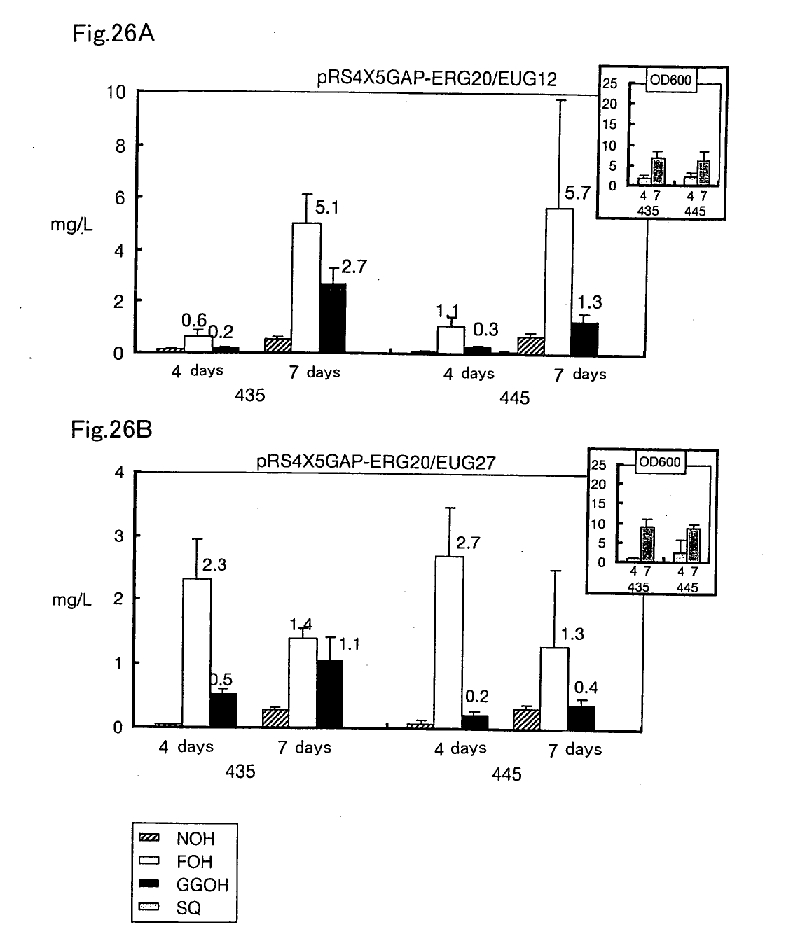

Subsequently, the effect of the expression enhancement of ERG20 upon YPH499-derived EUG12 and YPH500-derived EUG27 was examined. Each 5 colonies of these EUG strains into which pRS435GAP-ERG20 or pRS445GAP-ERG20 had been introduced were cultured. Then, mean values of their prenyl alcohol yields and standard deviations were recorded in graphs. The effect of ERG20 expression on prenyl alcohol production was as follows. When pRS435GAP-ERG20 was introduced into EUG12 and EUG27, recombinants produced GGOH at 2.7 mg/L on the average (3.6 mg/L at the maximum) and 1.1 mg/L on the average (1.37 mg/L at the maximum), respectively, when cultured for 7 days (FIGS. 26A, B). Since ERG20 encodes an FPP synthase gene, this fact that the expression enhancement of ERG20 led to production of GGOH suggests that, actually, GGPP synthase uses not DMAPP but FPP as a homoallylic primer substrate and that GGPP production ability was increased as a result of increased intracellular FPP synthesis activity caused by the ERG20 expression enhancement. Thus, it was demonstrated that ERG20 is effective in GGOH production in EUG strains.

(6-4) Prenyl Alcohol Production by BTS1 Expression Strains

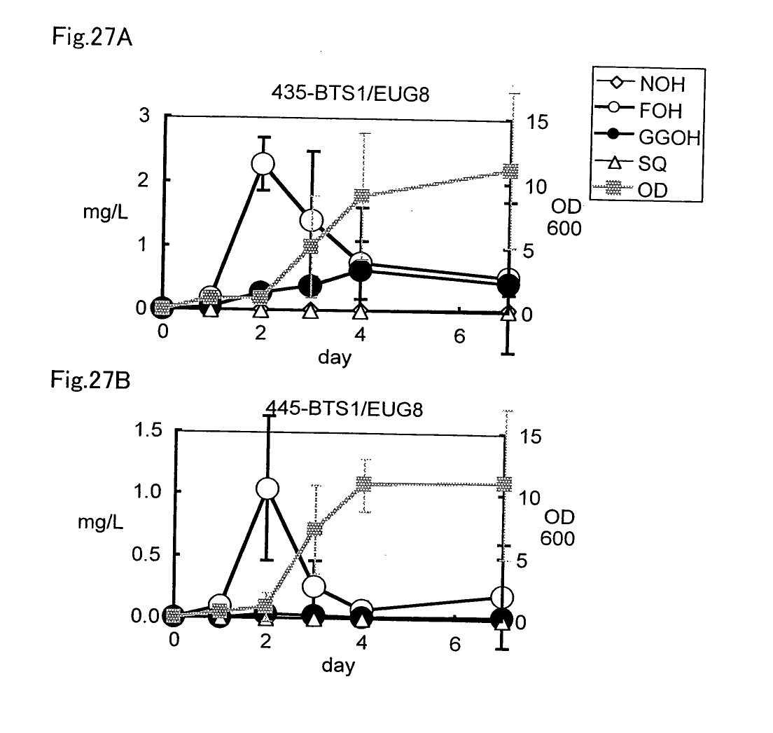

Each 5 colonies of pRS435GAP-BTS1/EUG8 (FIG. 27A) and pRS445GAP-BTS1/EUG8 (FIG. 27B) were cultured. Then, mean values of their prenyl alcohol yields and standard deviations were recorded in graphs. The effect of BTS1 expression on prenyl alcohol production was as follows. When pRS435GAP-BTS1 was introduced, GGOH productivity was improved to about 0.5 mg/L on the average (the maximum yield was 1.42 mg/L). From the results of preliminary experiments, it was already found that even the introduction of pRS435GAP-BTS1 into A451 leads to GGOH productivity of about 0.1-0.3 mg/L. Now, it was found that the introduction of the same plasmid is also effective on GGOH production when EUG8 is used as a host.

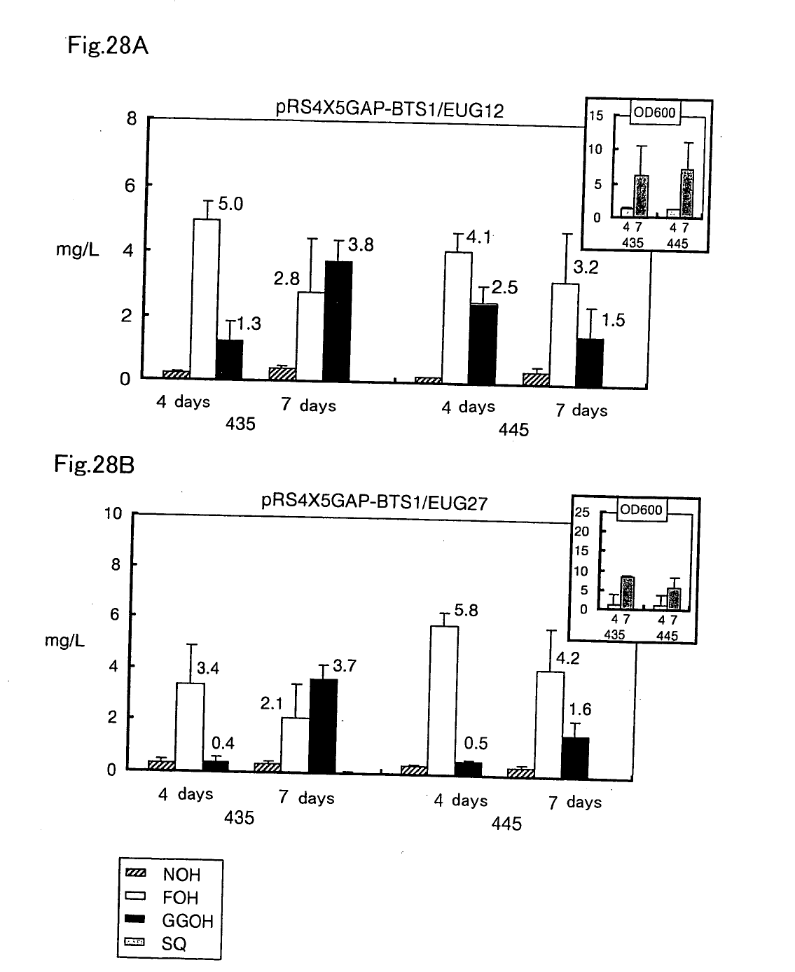

Subsequently, the effect of the expression enhancement of BTS1 upon YPH499-derived EUG12 and YPH500-derived EUG27 was examined. Each 5 colonies of these EUG strains into which pRS435GAP-BTS1 or pRS445GAP-BTS1 had been introduced were cultured. Then, mean values of their prenyl alcohol yields and standard deviations were graphed. The effect of BTS1 expression on prenyl alcohol production was as follows. When pRS435GAP-BTS1 was introduced, GGOH productivity of about 3.7-3.8 mg/L on the average was observed when recombinants were cultured for 7 days (maximum yields were 4.2 mg/L and 4.4 mg/L, respectively) (FIG. 28A, B). From the results of preliminary experiments, it was already found that even the introduction of pRS435GAP-BTS1 into YPH499 and YPH500 leads to GGOH productivity of about 0.1-0.2 mg/L. Now, it was found that the introduction of the same plasmid into EUG12 and EUG27 is more effective on GGOH production than the introduction into conventional hosts for recombination.

From the results so far described, it is understood that (i) mutating a cell so that an amount of squalene synthase gene transcript having translational activity can be reduced, to thereby prepare a mutant cell of the invention (e.g., EUG strains) without deleting its squalene synthase gene completely or partially; (ii) preparing a recombinant by introducing into the above mutant cell a recombinant DNA for expression or a DNA for genomic integration comprising an IPP biosynthetic pathway-related enzyme (e.g., HMG1, ERG20 or GTS1); and (iii) culturing the recombinant can lead to construction of a system capable of producing prenyl alcohol as a result of reduction in the amount of the squalene synthase gene transcript having translational activity.

### EXAMPLE 8

### Prenyl Alcohol Production by Fusion Gene-Transferred EUG Strains

An unexpected result was obtained in Example 7 that the introduction of FPP synthase gene into EUG strains improves GGOH productivity. This suggests that the GGPP synthase encoded by S. cerevisiae BTS1 prefers FPP to DMAPP as a primer substrate. Therefore, it was believed that enforcement of FPP synthesis ability is also required for further enforcing the ability to synthesize GGPP (a precursor of GGOH) from IPP.

In view of this, fusion genes composed of BTS1 and ERG20 were created in this Example.

In this Example, it was attempted to express the fusion gene in S. cerevisiae cells and to ascertain whether GGOH productivity improves or not. Further, it was also attempted to create mutants of BTS1, ERG20 and their fusion genes in which a nucleotide sequence encoding an ER signal is located at the 3′end and to examine the effect of introduction of these mutants upon prenyl alcohol production.

(1) Preparation of Plasmid DNA for Fusion Gene Transfer

A series of PCR reactions were carried out using pYESGGPS, which is a pYES2 plasmid incorporating GGPP synthase gene BTS1, and pT7ER20, which is a pT7 plasmid incorporating FPP synthase gene ERG20, as a template. The PCR primers used are as follows.

SacII-BTS1:

(SEQ ID NO: 70)

5′-TCC

CCG CGG@ ATG GAG GCC AAG ATA GAT-3′
BTS1-Xhol:

(SEQ ID NO: 71)

5′-CAA CTC GAG@ TCA CAA TTC GGA TAA GTG-3′
ERG20HDEL-XbaI:

(SEQ ID NO: 72)

5′-GCT CTA GA@G TTC GTC GTG TTT GCT TCT CTT GTA
AACTT-3′
BTS1HDEL-XhoI:

(SEQ ID NO: 73)

5′-TAT CTC GAG TCA CAA TTC GTC ATG TAA ATT GG-3′
BTS1-109I

(SEQ ID NO: 74)

5′-GCA GGG ACC CCA ATT CGG ATA AGT GGT C-3′
109I-BTS1:

(SEQ ID NO: 75)

5′-GTA GGG TCC CTG GAG GCC AAG ATA GAT G-3′
ERG20-109I:

(SEQ ID NO: 76)

5′-GCA GGG ACC CTT TGC TTC TCT TGT AAA CT-3′
109I-ERG20:

(SEQ ID NO: 77)

5′-GTA GGG TCC TCA GAA AAA GAA ATT AGG AG-3′
−21:

(SEQ ID NO: 78)

5′-TGT AAA ACG ACG GCC AGT-3′
T7:

(SEQ ID NO: 79)

5′-TAA TAC GAC TCA CTA TAG GG-3′

The nucleotides from position 3 to position 8 of ERG20HDEL-XbaI and the nucleotides from position 4 to position 9 of BTS1HDEL-XhoI (both 6 nucleotides are underlined) represent the SacII, XhoI or XbaI recognition site for vector ligation. The nucleotide from position 4 to position 10 of BTS1-109I, 109I-BTS1, ERG20-109 I and 109I-ERG20 (individual 7 nucleotides are underlined) represent the EcoO109 I recognition site for fusion gene preparation.

The PCR was carried out in the following reaction solution.

1x KOD-Plus buffer (Toyobo)
0.2 mM dNTPs
0.25 mM MgSO₄
15 pmol Primer 1
15 pmol Primer 2
0.01-0.1 μg Template DNA
1 unit KOD-Plus DNA polymerase (Toyobo)
Total: 50 μl

KOD-Plus contains 1.6 μg/μl of KOD antibody. Following an initial denaturation of 2 min at 94° C., the PCR was carried out for 30 cycles each consisting of 15 sec at 94° C., 30 sec for 55° C. and 1 min at 68° C. Then, the solution was retained at 68° C. for 2 min.

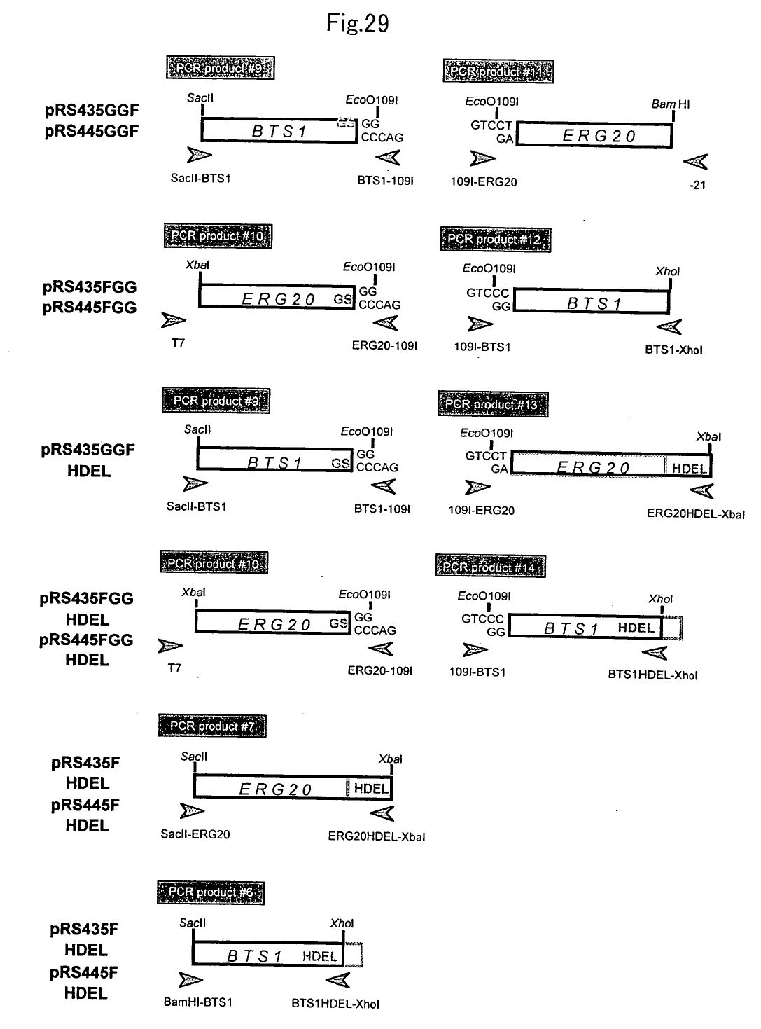

1st PCR reactions were carried out using the combinations of a template and a pair of primers (Primer 1, Primer 2) as indicated in Table 6 and FIG. 29. The designations of PCR products are also shown in Table 6 and FIG. 29. In FIG. 29, the final designations of plasmids are shown in the leftmost column. The sequences written in gray letters represent amino acids. Of these amino acids, GS was introduced into the binding sequence of the fusion gene, and HDEL was located at the 3′ end of the fusion gene by insertion or deletion mutation. Each arrowhead indicates the location and direction of each primer used in the PCR.

TABLE 6

| Template | primer 1 | primer 2 | PCR Product |
|---|---|---|---|
| pT7ERG20 | SacII-BTS1 | BTS1HDEL-Xho1 | #6 |
| pYESGGPS | SacII-ERG20 | ERG20HDEL-XbaI | #7 |
| pYESGGPS | SacII-BTS1 | BTS1-109I | #9 |
| PT7ERG20 | T7 | ERG20-109I | #10 |
| PT7ERG20 | 109I-ERG20 | −21 | #11 |
| pYESGGPS | 109-BTS1 | BTS1-XhoI | #12 |
| PT7ERG20 | 109I-ERG20 | ERG20HDEL-XbaI | #13 |
| pYESGGPS | 109I-BTS1 | BTS1HDEL-Xho1 | #14 |

PCR products #9, #10, #11, #12, #13 and #14 were digested with the restriction enzyme Eco0109I. Then, #9 and #11, #10 and #12, #9 and #13, and #10 and #14 were ligated to each other individually. Using the resultant ligation solutions as a template, 2nd PCR reactions were carried out under the same conditions as in the 1st PCR. As primers, the combinations of SacII-BTS1 and −21, T7 and BTS1-XhoI, SacII-BTS1 and ERG20HDEL-XbaI, and T7 and BTS1HDEL-XhoI were used, respectively. As a result, 2nd PCR products #9-#11, #10-#12, #9-#13 and #10-#14 were obtained.

The product #9-#11 was digested with SacII and BamHI, and inserted into the SacII-BamHI site of pRS435GAP and pRS445GAP to obtain pRS435GGFand pRS445GGF, respectively.

The product #10-#12 was digested with XbaI and XhoI, and inserted into the XbaI-XhoI site of pRS435GAP and pRS445GAP to obtain pRS435FGG and pRS445FGG, respectively.

The product #9-#13 was digested with SacII and XbaI, and inserted into the SacII-XbaI site of pRS435GAP to obtain pRS435GGFHDEL.

The product #10-#14 was digested with XbaI and XhoI, and inserted into the XbaI-XhoI site of pRS435GAP and pRS445GAP to obtain pRS435FGGHDEL and pRS445FGGHDEL, respectively.

The product #7 was digested with SacII and XbaI, and inserted into the SacII-XbaI site of pRS435GAP and pRS445GAP to obtain pRS435FHDEL and pRS445FHDEL, respectively.

The product #6 was digested with BamHI and XhoI, and inserted into the BamHI-XhoI site of pRS435GAP and pRS445GAP to obtain pRS435GGHDEL and pRS445GGHDEL, respectively.

It was confirmed by DNA sequencing that each of the resultant plasmid DNAs has the exact nucleotide sequence as designed. BTS1-ERG20 fusion gene was designated GGF; ERG20-BTS1 fusion gene was designated FGG; a gene recombined so that a sequence encoding HDEL is located at the 3′ end of GGF was designated GGFHDEL; a gene recombined so that a sequence encoding HDEL is located at the 3′ end of FGG was designated FGGHDEL; and a gene recombined so that a sequence encoding HDEL is located at the 3′ end of BTS1 was designated GGHHDEL.

As plasmids for expressing non-fusion genes BTS1 and ERG20 separately, pRS435GAP-BTS1 (called pRS435GG), pRS445GAP-BTS1 (called pRS445GG), pRS435GAP-ERG20 (called pRS435F) and pRS445GAP-ERG20 (called pRS445F) were used. As plasmids for expressing HMG1, pRS434TEF-HMG1 and pRS434GAP-HMG1 were used.

(2) Preparation of Recombinants

Recombinants were prepared by introducing the plasmid prepared above into the host using Frozen EZ yeast transformation kit (Zymo Research, Orange, Calif.). As the host, EUS5 and EUG12 were used. As controls, A451 and YPH499 were used.

(3) Determination of Prenyl Alcohol Yields

A451 and YPH499 were inoculated into SD medium. EUG strains and recombinants created therefrom were inoculated into SGR medium. All of them were cultured at 30° C. to prepare preculture broth. Ten or 25 μl of the preculture broth was added to 1.0 or 2.5 ml of YM7+ade medium (YM, pH 7, 40 μg/ml adenine sulfate) or YMO7 medium [YM7+ade, 1% (w/v) soybean oil, 0.1% (w/v) Adekanol LG-109 (Asahi Denka Kogyo, Tokyo, Japan)] and cultured at 30° C. for 4 days or 7 days under reciprocal shaking at 130 rpm.

After completion of the cultivation, an equal volume of methanol was added to the culture broth and mixed. Approximately 2 volumes of pentane was added to this mixture, agitated vigorously and then left stationary. The resultant pentane layer was transferred into a fresh glass tube, which was then placed in a draft. Pentane was evaporated therein to condense the solute components. Subsequently, prenyl alcohols were identified and quantitatively determined by GC/MS using undecanol as an internal standard for quantitative determination. At that time, the degree of cell growth was also examined by diluting 20 μl of the culture broth 30-fold with water and measuring the absorbance at 600 nm.

The yields of prenyl alcohols were determined with PH6890/5973 GC/MS system in the same manner as described in (1-2) in Example 2.

(4) Results and Considerations

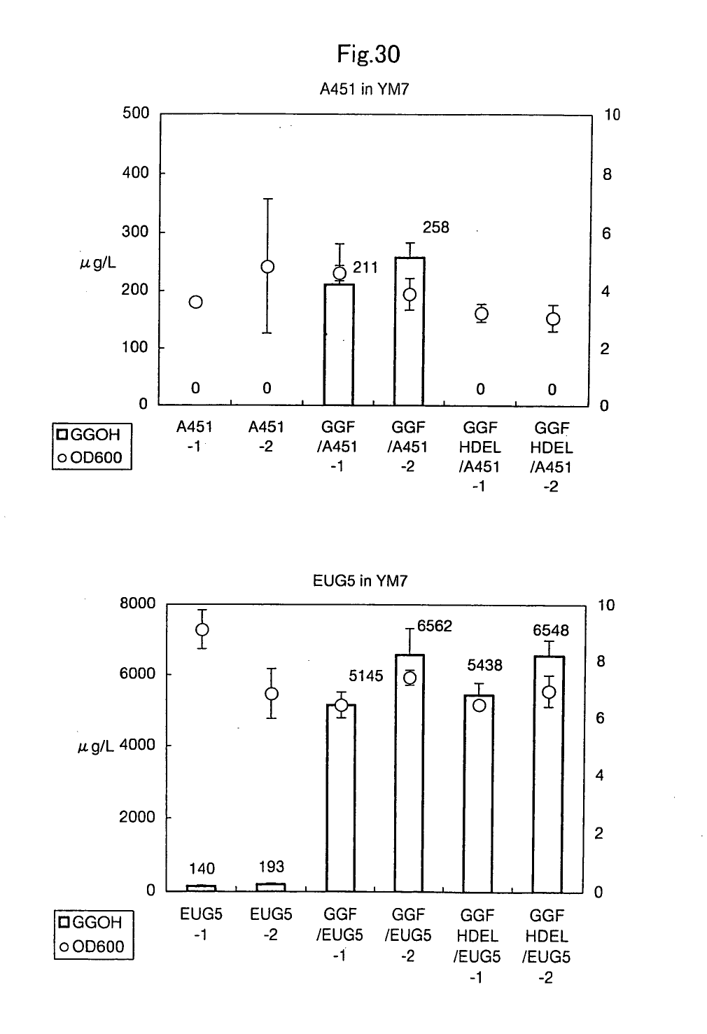

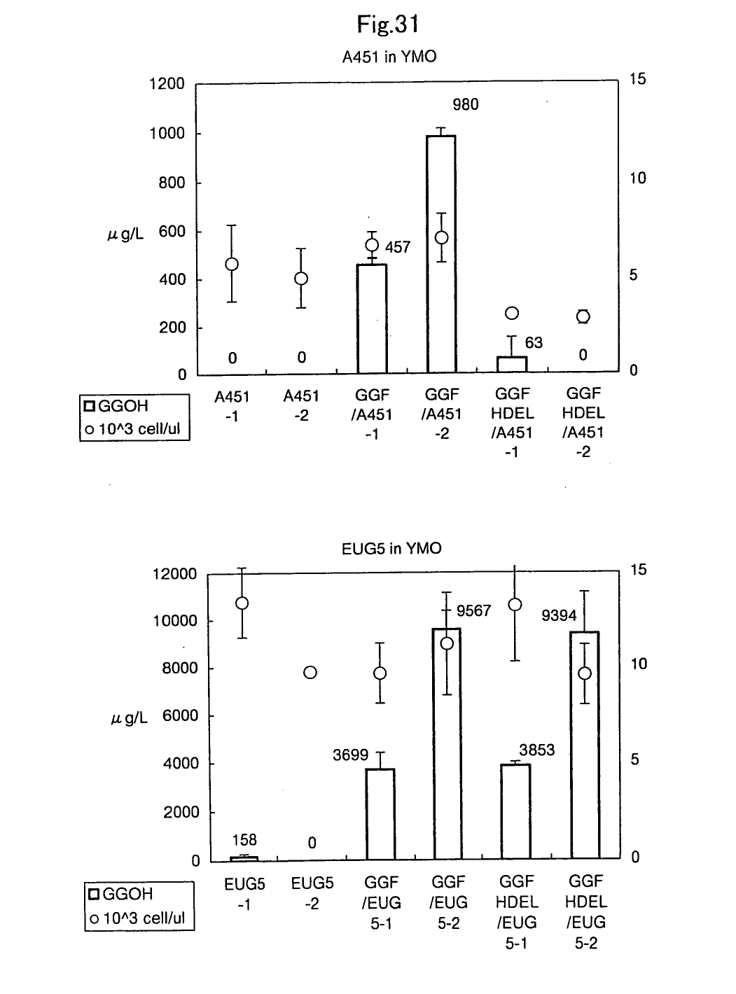

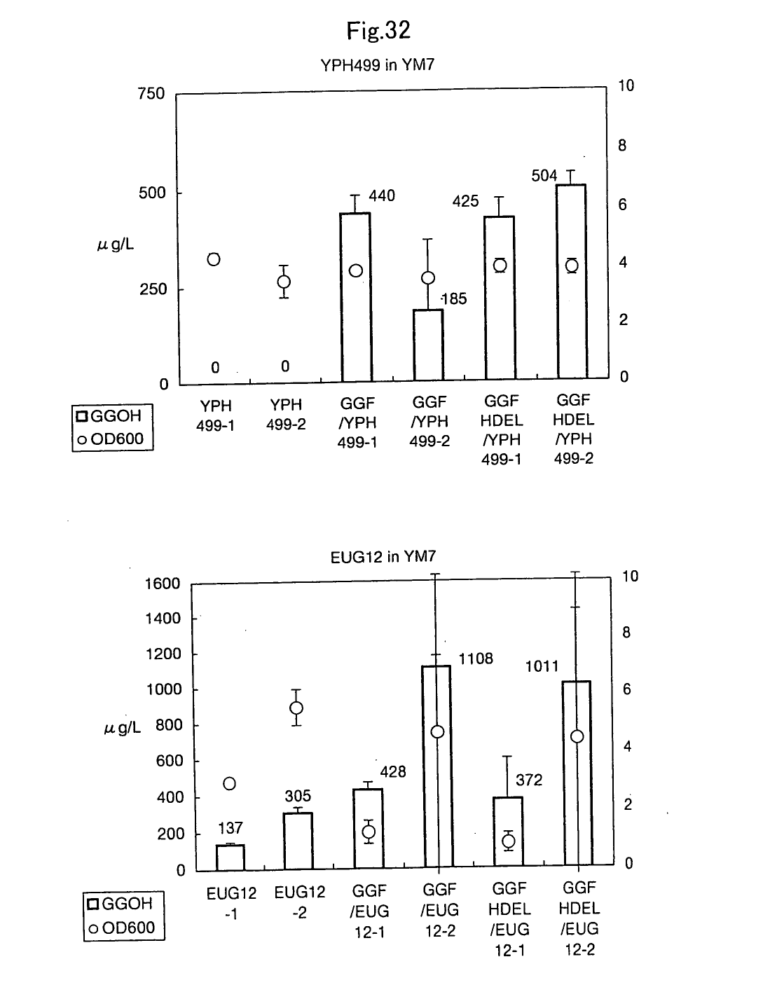

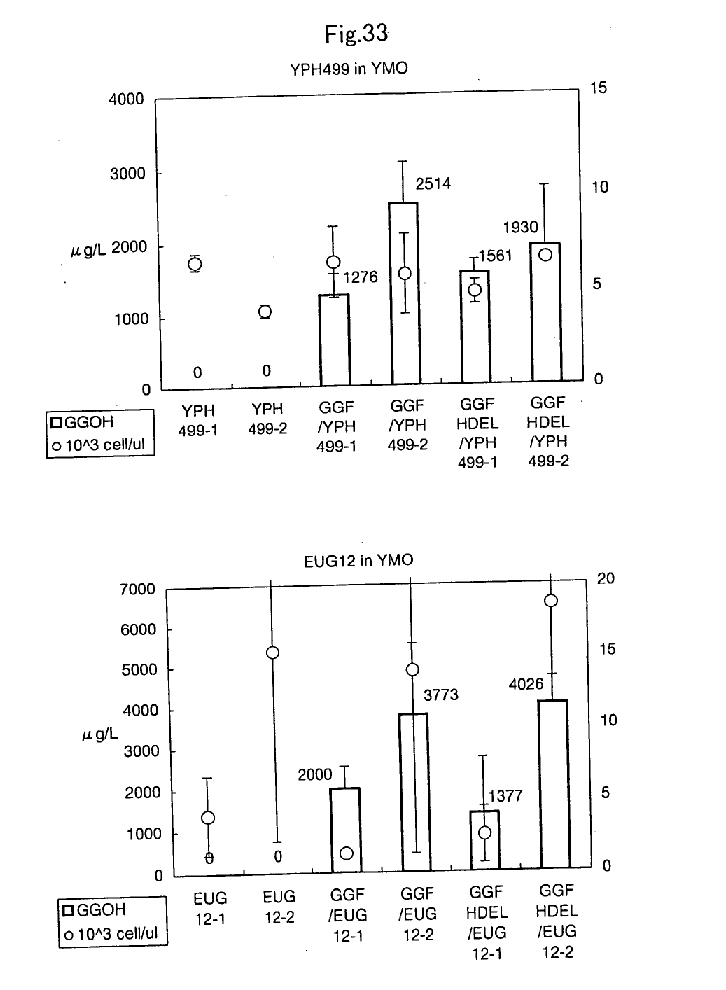

Each of the recombinants prepared was cultured in YM7 medium and YMO7 medium for 4 to 7 days, followed by determination of prenyl alcohol yields. The results obtained using A451-derived EUG strains as hosts are shown in FIGS. 30 and 31. The results obtained using YPH499-derived EUG strains as hosts are shown in FIGS. 32 and 33. In FIGS. 30 and 31, “GGFHDEL” represents pRS435GGFHDEL. “−1” represents the yield when cells were cultured for 4 days, and “−2” represents the yield when cells were cultured for 7 days. Since cells are suspended in soybean oil in YMO7 medium, the amount of cells is expressed as the number of cells. “10\hat{ }3 cell/μl” represents a value obtained by dividing the number of cells per microliter by 1000.

pRS435GGF/A451 produced 0.26 mg/L of GGOH on the average (0.28 mg/L at the maximum) when cultured for 7 days in YM7 medium (FIG. 30; upper panel; GGF/A451-2), and produced 0.98 mg/L on the average (1.0 mg/L at the maximum) when cultured for 7 days in YMO7 medium (FIG. 31; upper panel; GGF/A451-2).

On the other hand, when EUG5 was used as the host, pRS435GGF-transferred recombinants produced 6.6 mg/L of GGOH on the average (maximum: 7.3 mg/L) when cultured for 7 days in YM7 medium (FIG. 30; lower panel; GGF/EUG5-2), and produced 9.6 mg/L of GGOH on the average (maximum: 10.1 mg/L) when cultured for 7 days in YMO7 medium (FIG. 31; lower panel; GGF/EUG5-2).

pRS435GGF-transferred YPH499 produced 0.19 mg/L of GGOH on the average (0.37 mg/L at the maximum) when cultured for 7 days in YM7 medium (FIG. 32; upper panel; GGF/YPH499-2), and produced 2.5 mg/L on the average (2.9 mg/L at the maximum) in YMO7 medium (FIG. 33; upper panel; GGF/YPH499-2). On the other hand, when YPH499-derived EUG12 was used as the host, the pRS435GGF- or pRS435GGFHDEL-transferred clones produced 3.7-4.0 mg/L of GGOH on the average (5.4-5.8 mg/L at the maximum) when cultured for 7 days in YMO medium (FIG. 33; lower panel; GGF/EUG12-2 and GGFHDEL/UG12-2). Thus, it was demonstrated that recombinants prepared from the mutant cell of the invention (such as EUG5 or EUG12) have higher GGOH productivity than those recombinants prepared from conventional hosts for recombination (such as YPH499 or A451). As described earlier, the mutant cell of the invention is engineered so that an amount of squalene synthase gene transcript having translational activity can be reduced without total or partial deletion of the squalene synthase gene.

### EXAMPLE 9

### Prenyl Alcohol Production by Gene-Transferred EUG in Media with Various Glucose-Galactose Compositions

(1) Introduction of Vectors into Hosts and Their Cultivation

In this Example, how the prenyl alcohol production by budding yeast will change when Glc-Gal sugar composition is varied is examined. In addition, the effect of the expression of BTS1-ERG20 fusion genes upon prenyl alcohol production is also examined.

Vectors were introduced into yeast hosts using Frozen EZ yeast transformation II kit purchased from Zymo Research (Orange, Calif.). As BTS1-ERG20 fusion gene-expression plasmids, pRS435GGF and pRS435GGFHDEL were used. As hosts, EUG5 and EUG12 were used. As control hosts, A451 and YPH499 were used.

Each of the recombinants obtained was precultured in SGR selection medium. Then, 0.01-0.05 ml of the preculture broth was added to 1-5 ml of the YM7 medium described below and cultured in a test tube 18 mm in diameter at 30° C. under reciprocal shaking at 130 rpm. Five types of YM7 medium having the following sugar components (Glc-Gal composition ratios), respectively, were prepared in advance: 0% Glc-100% Gal, 20% Glc-80% Gal, 50% Glc-50% Gal, 75% Glc-25% Gal, and 100% Glc-0% Gal. First, cells were cultured in these media at 30° C. under reciprocal shaking at 130 rpm. Two days after the start of cultivation, Glc was added further to each medium to give a final concentration of 5% (w/v). Cells were cultured further up to day 7.

(2) Results and Considerations

(2-1) GGOH Production by EUG5

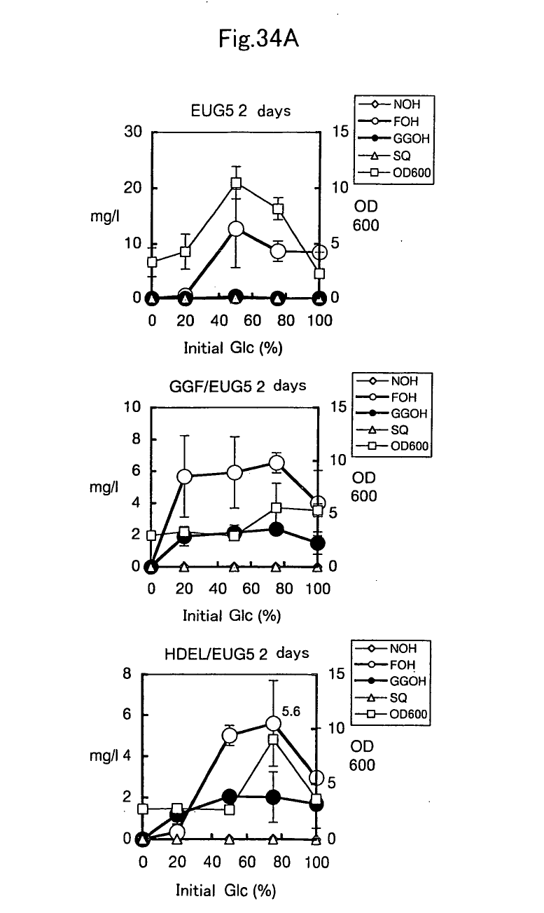

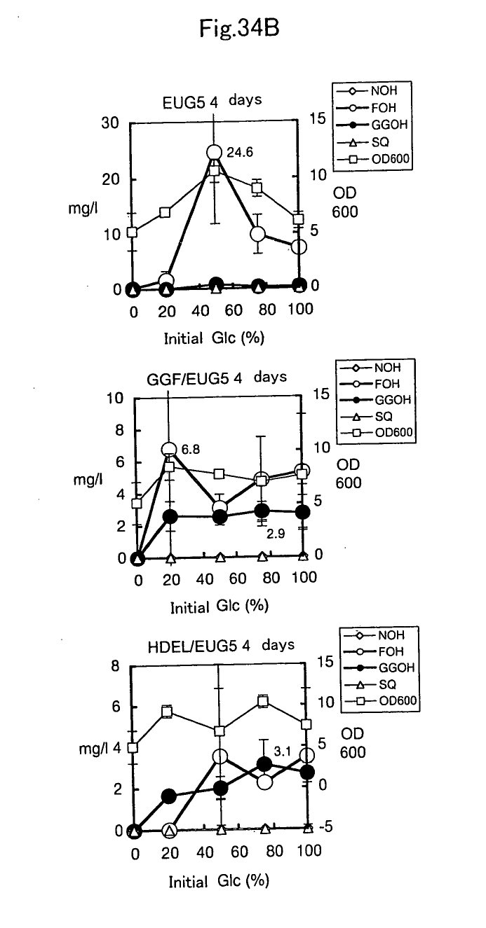

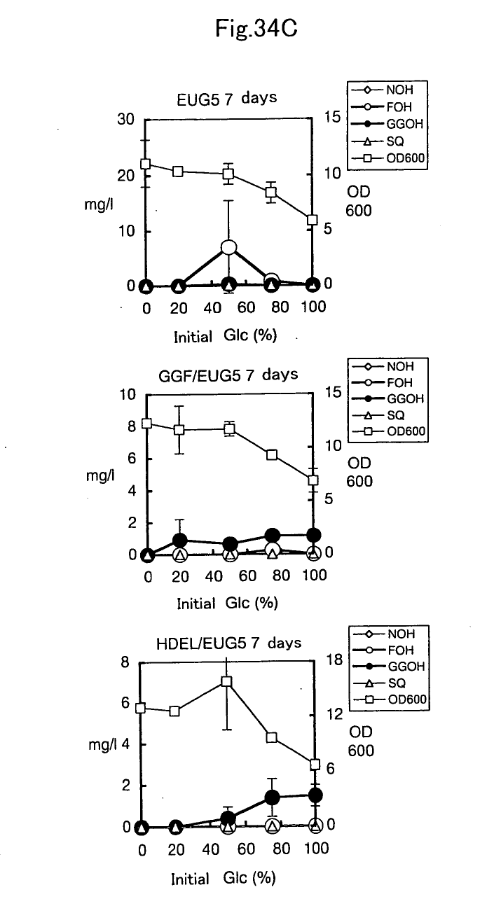

FIG. 34 shows the results of GGOH production when pRS435GGF or pRS435GGFHDEL was transferred into EUG5. In both pRS435GGF-transferred clone (expressed as “GGF/EUG5” in FIG. 34) and pRS435GGFHDEL-transferred clone (expressed as “HDEL/DUG5” in FIG. 34), good results were obtained after 2-4 days cultivation when the initial Glc was 20-80%.

(2-2) GGOH Production by EUG12

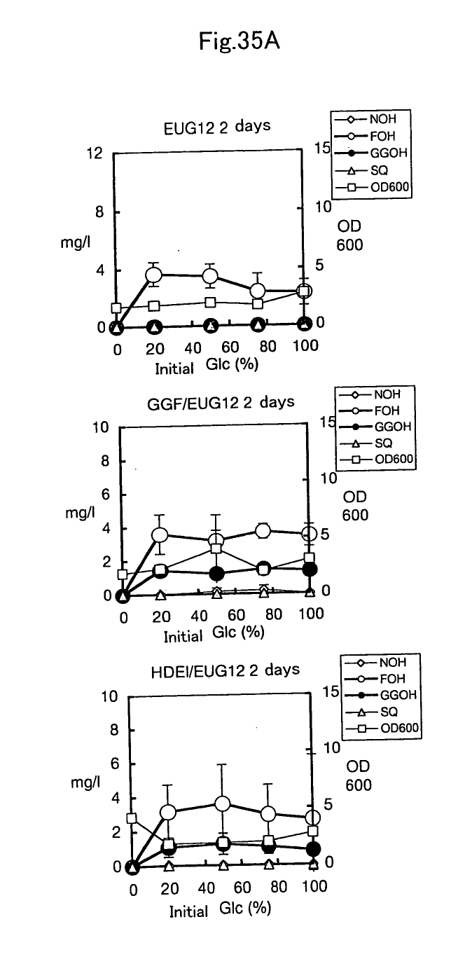

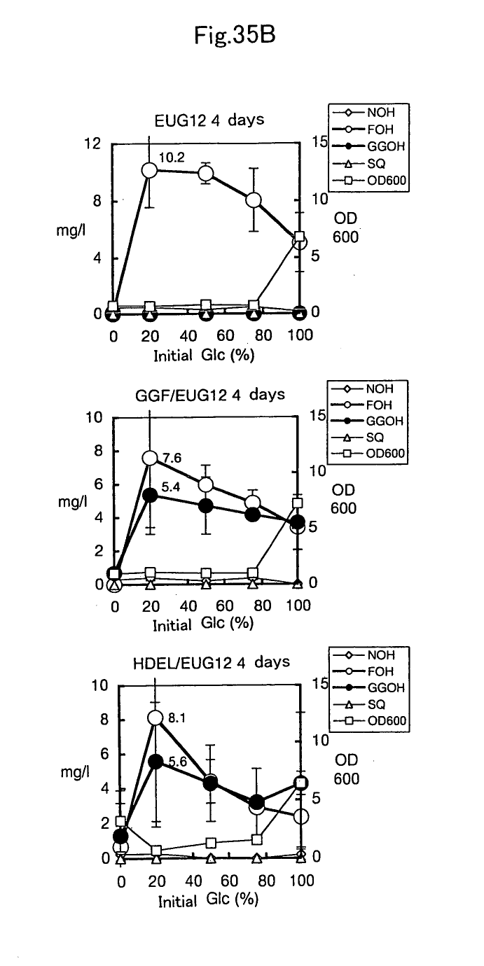

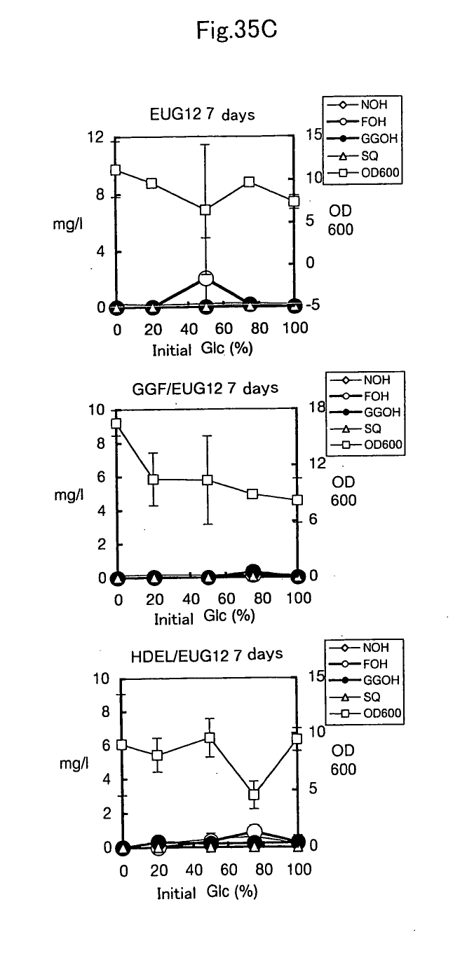

FIGS. 35A-C shows the results of GGOH production when pRS435GGF or pRS435GGFHDEL was transferred into EUG12. When the initial Glc was 20%, both of the recombinants exhibited high prenyl alcohol productivity. When the initial Glc was 20%, pRS435GGF/EUG12 (expressed as “GGF/EUG12” in FIG. 35) and pRS435GGFHDEL/EUG12 (expressed as “HDEL/EUG12” in FIG. 35) produced FOH at 7.6 mg/L on the average (10.5 mg/L at the maximum) and at 8.1 mg/L on the average (12.6 mg/L at the maximum), respectively, when cultured for 4 days, even when the amounts of their cells corresponded to OD₆₀₀=1.1 and 0.70, respectively (data not shown). On the other hand, average GGOH yields of these recombinants were 5.4 mg/L (7.0 mg/L at the maximum) and 5.6 mg/L (8.0 mg/L at the maximum), respectively. It is believed that these production results are very efficient as productivity per cell.

### EXAMPLE 10

### Prenyl Alcohol Yields in YPD07rich Medium

(1) Cultivation and Determination of Prenyl Alcohol Yields

EUG5, EUG24, EUG36 and EUG64 prepared in Example 1; recombinants prepared by introducing pRS434-GAP-HMG1 into these EUG strains; and recombinants prepared by further introducing pRS435GG into each of the resultant recombinants were cultured in YPD07rich medium [YPD, 1% (v/v) soybean oil, 0.1% (v/v) Adekanol LG-109, 5% (w/v) Glc (final concentration 7% (w/v) Glc), pH 7] at 30° C. under reciprocal shaking at 130 rpm. Then, prenyl alcohol yields were determined in the same manner as described in Example 2.

(2) Results and Considerations

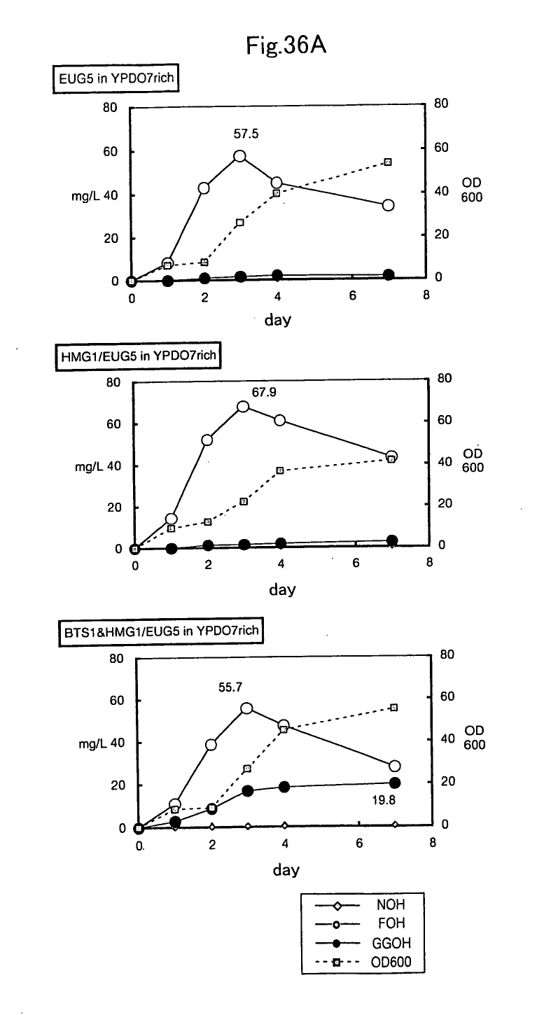

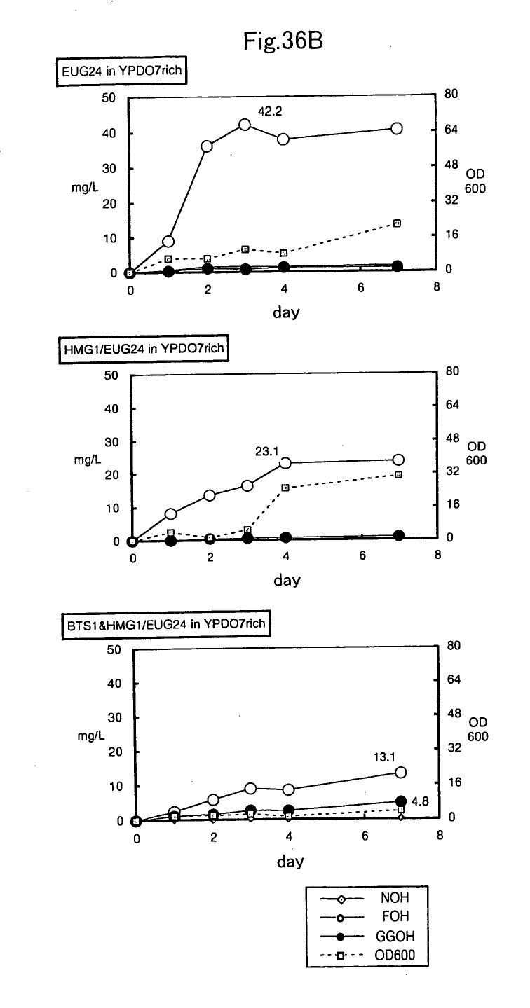

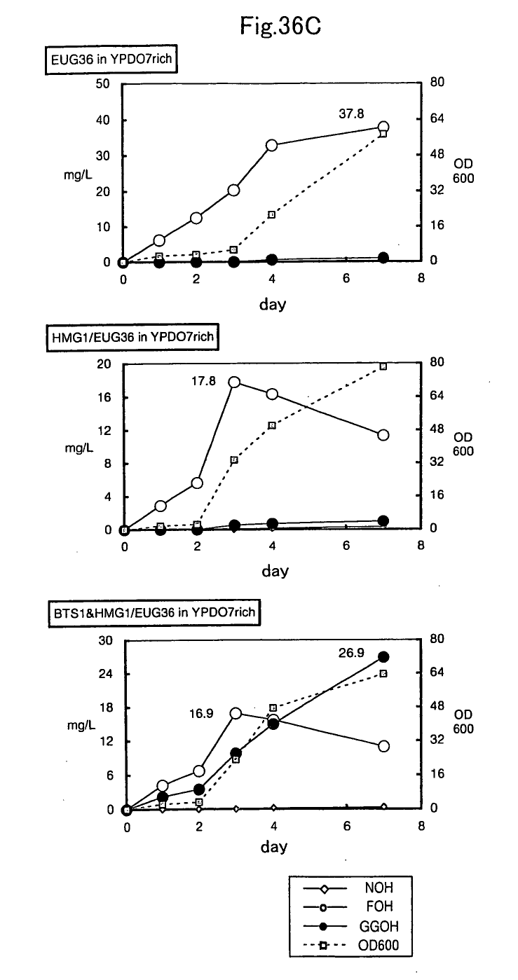

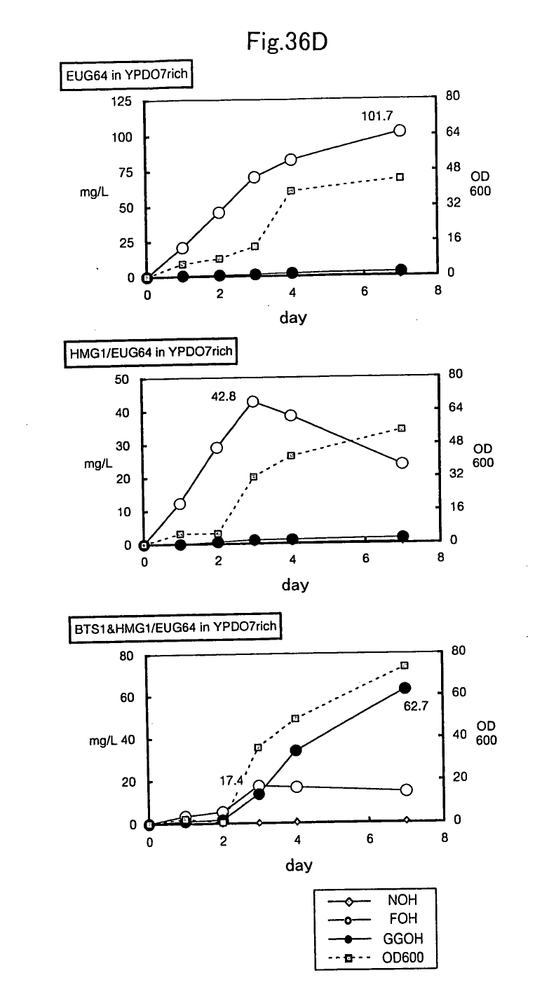

FIGS. 36A-D shows the amounts of prenyl alcohols accumulated in the culture broth and the absorbance of the broth at 600 nm (OD₆₀₀) arranged in graphs. By culturing the cells in YPD07rich medium containing nitrogen sources and sugar sources at higher concentrations than YM medium, prenyl alcohols of still higher concentrations could be produced.

When EUG5 was cultured in YPDO7rich medium for 3 days, this strain produced 57.5 mg/L of FOH and 1.70 mg/L of GGOH (FIG. 36A, “EUG5 in YPDO7rich”). FUG24 produced 42.2 mg/L and 0.87 mg/L, respectively, when cultured for 3 days (FIG. 36B, “EUG24 in YPDO7rich”). FUG36 produced 37.8 mg/L and 0.98 mg/L, respectively, when cultured for 7 days (FIG. 36C, “EUG36 in YPDO7rich”). FUG64 produced 101.7 mg/L and 2.92 mg/L, respectively, when cultured for 7 days (FIG. 36D, “EUG64 in YPDO7rich”). Thus, it has become clear that a system capable of producing 0.1 g or more prenyl alcohols per liter of culture broth can be constructed by using the mutant cell of the invention (e.g., EUG64) as a host, preparing a recombinant by introducing into the mutant cell a recombinant DNA for expression or a DNA for genomic integration comprising an IPP biosynthetic pathway-related gene, and culturing the recombinant, and reducing an amount of squalene synthase gene transcript having transcriptional activity. The system, unlike simple squalene synthase gene-deficient strains such as S. cerevisiae ATCC64031, does not require addition of sterols to the medium caused by ergosterol requirement.

With respect to HMG1-transferred recombinants, the recombinant from EUG5 produced 67.9 mg/L of FOH when cultured for 3 days (FIG. 36A, “HMG1/EUG5 in YPDO7rich”), but the FOH productivity of other recombinants was not improved compared to the productivity of their host strains.

When BTS1 was further introduced into HMG1-transferred EUG5, the recombinant exhibited high production of GGOH while retaining the FOH production of the host. The recombinant produced 16.7 mg/L of GGOH when cultured for 3 days, and 19.8 mg/L of GGOH when cultured for 7 days (FIG. 36A, “BTS1&HMG1/EUG5 in YPDO7rich”). Remarkably, the recombinant created from EUG64 produced 62.7 mg/L of GGOH when cultured for 7 days. This means that when a recombinant is prepared by introducing into the mutant cell of the invention (e.g., EUG64) a recombinant DNA for expression or a DNA for genomic integration each comprising an IPP biosynthetic pathway-related gene and then cultured, a system can be constructed which is capable of producing such prenyl alcohols (e.g., GGOH) that are not detected in the culture broth of simple squalene synthase gene-deficient strains such as S. cerevisiae ATCC64031, at a high level exceeding 0.06 g per liter of the culture broth as a result of reduction of the squalene synthase gene transcript having translational activity.

All publications, patents and patent applications cited herein are incorporated herein by reference in their entirety.

### INDUSTRIAL APPLICABILITY

According to the present invention, there are provided methods of producing various prenyl alcohols having geometrical isomerisms of naturally occurring prenyl alcohols. Since active prenyl alcohols having geometrical isomerisms of naturally occurring prenyl alcohols can be obtained in large quantities according to the invention, they are applicable to industrial production of huge varieties of isoprenoid/terpenoid compounds important in vivo, and also applicable as basic systems to find out novel physiological activities of active prenyl alcohols. Thus, the methods of the invention are useful.

SEQUENCE LISTING FREE TEXT

| | |
|---|---|
| SEQ ID NOS: 34-79: | synthetic DNA |

1. A method of producing a prenyl alcohol(s), comprising culturing a mutant cell that has been mutated so that an amount of squalene synthase gene transcript having translational activity can be reduced, and recovering the prenyl alcohol(s) from the resultant culture.

2. A method of producing a prenyl alcohol(s), comprising preparing a recombinant by introducing a recombinant DNA for expression or a DNA for genomic integration each comprising an IPP biosynthetic pathway-related enzyme gene into a mutant cell that has been mutated so that an amount of squalene synthase gene transcript having translational activity can be reduced, culturing the resultant recombinant, and recovering the prenyl alcohol(s) from the resultant culture.

3. A method of producing a prenyl alcohol(s), comprising culturing a mutant cell in which the transcription promoter region of its squalene synthase gene has been replaced with a transcription repression-type promoter under transcription repression conditions; reducing an amount of squalene synthase gene transcript having translational activity; and recovering the prenyl alcohol(s) from the resultant culture.

4. A method of producing a prenyl alcohol(s), comprising preparing a recombinant by introducing a recombinant DNA for expression or a DNA for genomic integration each comprising an IPP biosynthetic pathway-related enzyme gene into a mutant cell in which the transcription promoter region of its squalene synthase gene has been replaced with a transcription repression-type promoter so that an amount of squalene synthase gene transcript having translational activity can be reduced, culturing said recombinant under transcription repression conditions; and recovering the prenyl alcohol(s) from the resultant culture.

5. The method according to claim 3 or 4, wherein the transcription repression-type promoter is GAL1 promoter.

6. The method according to claim 3 or 4, wherein the transcription repression conditions comprise the use of a glucose-containing medium.

7. The method according to claim 2 or 4, wherein the IPP biosynthetic pathway-related enzyme gene is any one selected from the group consisting of the following genes (a) through (l):
(a) farnesyl diphosphate synthase gene
(b) geranylgeranyl diphosphate synthase gene
(c) hydroxymethylglutaryl-CoA reductase gene
(d) isopentenyl diphosphate Δ-isomerase gene
(e) mevalonate kinase gene
(f) acetyl-CoA acetyltransferase gene
(g) hydroxymethylglutaryl-CoA synthase gene
(h) phosphomevalonate kinase gene
(i) diphosphomevalonate decarboxylase gene
(j) a mutant of any one of the above genes (a) through (i)
(k) a fusion gene composed of a gene selected from the group consisting of the above genes (a) through (i) or a mutant thereof, and other gene or a mutant thereof
(l) a gene obtained by introducing an addition, substitution or insertion mutation into any one of the above genes (a) through (k) so that the polypeptide encoded by the resultant gene contains an endoplasmic reticulum signal.

8. The method according to claim 7, wherein the endoplasmic reticulum signal is represented by the sequence shown in SEQ ID NO: 30, 31 or 32.

9. The method according to any one of claims 1 to 8, wherein the cell is yeast.

10. The method according to claim 9, wherein the yeast is Saccharomyces cerevisiae.

11. The method according to claim 10, wherein the Saccharomyces cerevisiae is Saccharomyces cerevisiae A451 strain, YPH499 strain, YPH500 strain, W303-1A strain or W303-1B strain.

12. The method according to any one of claims 1 to 11, wherein the prenyl alcohol(s) are/is farnesol, nerolidol and/or geranylgeraniol.

13. The method according to any one of claims 3 to 12, wherein the cell is cultured under non-transcription repression conditions prior to the cultivation under transcription repression conditions.

14. The method according to claim 13, wherein the transcription repression conditions comprise the use of a glucose-containing medium and the non-transcription repression conditions comprise the use of a galactose-containing medium.

15. The method according to claim 7, wherein the farnesyl diphosphate synthase gene encodes the amino acid sequence as shown in SEQ ID NO: 2 or 4.

16. The method according to claim 7, wherein the geranylgeranyl diphosphate synthase gene encodes the amino acid sequence as shown in SEQ ID NO: 6.

17. The method according to claim 7, wherein the hydroxymethylglutaryl-CoA reductase gene encodes the amino acid sequence as shown in SEQ ID NO: 8.

18. The method according to claim 7, wherein the mutant of hydroxymethylglutaryl-CoA reductase gene comprises the nucleotide sequence as shown in any one of SEQ ID NOS: 11, 13 and 15 through 24.

19. The method according to claim 7, wherein the diphosphomevalonate decarboxylase gene encodes the amino acid sequence as shown in SEQ ID NO: 10.

20. A mutant cell that has been mutated so that an amount of squalene synthase gene transcnpt having translational activity can be reduced.

21. A mutant cell in which the transcription promoter region of its squalene synthase gene has been replaced with a transcription repression-type promoter so that an amount of transcript from its squalene synthase gene having translational activity can be reduced.
